SEQUENCE LISTING (14 sequences)

SEQ ID NO: 1 - protein, 376 aa, synthetic construct
  REGION 1..376: note = Synthetic: MAP kinase MPK4
MSAESCFGSSGDQSSSKGVATHGGSYVQYNVYGNLFEVSRKYVPPLRPIGRGAYGIVCAATNSETGEEVAIKKIGNAFDNIIDAKRTLREIKLLKHMDHENVIAVKDIIKPPQRENFNDVYIVYELMDTDLHQIIRSNQPLTDDHCRFFLYQLLRGLKYVHSANVLHRDLKPSNLLLNANCDLKLGDFGLARTKSETDFMTEYVVTRWYRAPELLLNCSEYTAAIDIWSVGCILGETMTREPLFPGKDYVHQLRLITELIGSPDDSSLGFLRSDNARRYVRQLPQYPRQNFAARFPNMSAGAVDLLEKMLVFDPSRRITVDEALCHPYLAPLHDINEEPVCVRPFNFDFEQPTLTEENIKELIYRETVKFNPQDSV

SEQ ID NO: 2 - protein, 372 aa, synthetic construct
  REGION 1..372: note = Synthetic: MAP kinase MPK12
MSGESSSGSTEHCIKVVPTHGGRYVQYNVYGQLFEVSRKYVPPIRPIGRGACGIVCAAVNSVTGEKVAIKKIGNAFDNIIDAKRTLREIKLLRHMDHENVITIKDIVRPPQRDIFNDVYIVYELMDTDLQRILRSNQTLTSDQCRFLVYQLLRGLKYVHSANILHRDLRPSNVLLNSKNELKIGDFGLARTTSDTDFMTEYVVTRWYRAPELLLNCSEYTAAIDIWSVGCILGEIMTGQPLFPGKDYVHQLRLITELVGSPDNSSLGFLRSDNARRYVRQLPRYPKQQFAARFPKMPTTAIDLLERMLVFDPNRRISVDEALGHAYLSPHHDVAKEPVCSTPFSFDFEHPSCTEEHIKELIYKESVKFNPDH

SEQ ID NO: 3 - protein, 390 aa, synthetic construct
  REGION 1..390: note = Synthetic: HT1 protein kinase polypeptide
MSGLCFNPFRLRWSLRSKLPLEPSLPNLPCNPSSSKTNRYAEAETMEKKRFDSMESWSMILESENVETWEASKGEREEWTADLSQLFIGNKFASGAHSRIYRGIYKQRAVAVKMVRIPTHKEETRAKLEQQFKSEVALLSRLFHPNIVQFIAACKKPPVYCIITEYMSQGNLRMYLNKKEPYSLSIETVLRLALDISRGMEYLHSQGVIHRDLKSNNLLLNDEMRVKVADFGTSCLETQCREAKGNMGTYRWMAPEMIKEKPYTRKVDVYSFGIVLWELTTALLPFQGMTPVQAAFAVAEKNERPPLPASCQPALAHLIKRCWSENPSKRPDFSNIVAVLEKYDECVKEGLPLTSHASLTKTKKAILDHLKGCVTSISSPFSSSSVPVNA

SEQ ID NO: 4 - DNA, 58 bp, synthetic construct
  misc_feature 1..58: note = Synthetic: exon sequence Bd 21-3
  source 1..58: mol_type = other DNA
gtcgccatcaagaagatcggcaacgcgttcgacaaccagatcgacgccaagcgcactt

SEQ ID NO: 5 - DNA, 58 bp, synthetic construct
  misc_feature 1..58: note = Synthetic: chill 1 sequence
  source 1..58: mol_type = other DNA
gtcgccatcaagaagatcggcaacgcgttcaacaaccagatcgacgccaagcgcactt

SEQ ID NO: 6 - DNA, 77 bp, synthetic construct
  misc_feature 1..77: note = Synthetic: Bd 21-3
  source 1..77: mol_type = other DNA
tgctgctatcaacgtgcagactcgcgaggaggtcgccatcaagaagatcggcaacgcgttcgacaaccagatcgacg

SEQ ID NO: 7 - DNA, 35 bp, synthetic construct
  misc_feature 1..35: note = Synthetic: CRISPR #1
  variation 27: note = n is a, c, g, or t
  source 1..35: mol_type = other DNA
tgctgctatcaacgtgcagactccccnaatagact

SEQ ID NO: 8 - DNA, 71 bp, synthetic construct
  misc_feature 1..71: note = Synthetic: CRISPR #2
  source 1..71: mol_type = other DNA
tgctgctatcaacgtgcagacaggaggacacgtcgccaacatcagcaacgcgttcgacttccacatccaca

SEQ ID NO: 9 - DNA, 25 bp, synthetic construct
  misc_feature 1..25: note = Synthetic: Chill1_CRISPR_Fw
  source 1..25: mol_type = other DNA
acttgtatcaacgtgcagactcgcg

SEQ ID NO: 10 - DNA, 25 bp, synthetic construct
  misc_feature 1..25: note = Synthetic: Chill1_CRISPR_Rv
  source 1..25: mol_type = other DNA
aaaccgcgagtctgcacgttgatac

SEQ ID NO: 11 - DNA, 1164 bp, synthetic construct
  misc_feature 1..1164: note = Synthetic: Bd21-3(WT) BdMPK5
  source 1..1164: mol_type = other DNA
atgcgcatggaaggcggcggcgccgggccggcggcagcagcagcagcaggaggagcccatggcctcggcgaggcgcagatcaagggcacgctcacccacggcggcaggtacgtgcagtacaacgtctacggcaacctcttcgaggtctccgccaagtacgtcccgcccatccgtcccgtcggccgcggcgcctgcggcatcatctgtgctgctatcaacgtgcagactcgcgaggaggtcgccatcaagaagatcggcaacgcgttcgacaaccagatcgacgccaagcgcactttgcgagaagtgaagctgcttcgacacatgaatcatgagaatgtgatttcaatcaaggacatcatacgcccgccaaggcgggagaattttaacgatgtttacatcgtttacgagctgatggacactgatcttcaccaccttcttagatcaaaccagccactcacagatgatcactgtcagtattttctctaccaagtgctcagaggattaaagtatgtgcactcggcgaatgtcttgcaccgagaccttaggccgagcaatctgttactgaatgccaaatgtgacctcaagattggggactttgggctggcgaggaccaccactgagactgacttcatgatggagtatgttgttacccggtggtatcgggcgccagagctccttcttaactgctcggagtacaccggagctattgatatgtggtcagtcggctgcatccttggtgagattgcaacaagagagcctctgtttcctggaaaagattacgttcatcagctgaggctaattaccgagctgctaggctcaccagacgacaccagcctagggtttcttcggagcgataatgcccgcagatatgtcaggtctcttcctcaataccccaagcaacaatttcgttcacggttcccaaatatgtccagtggcgccatggatttgcttgagaggatgcttgtatttgatccaaacaagaggattactgttgacgaggctctgtgccatccctacttggcatcccttcatgagataaacgatgaacctgtctgcccagcgcctttcagctttgatttcgagcagccatcatttaccgaggaagatatcaaagaactcatttggagggaatctgtcaagttcaaccctgaaccgattcactga

SEQ ID NO: 12 - DNA, 1098 bp, synthetic construct
  misc_feature 1..1098: note = Synthetic: chill1 BdMPK5
  variation 2..8: note = n is a, c, g, or t
  variation 10..11: note = n is a, c, g, or t
  variation 14: note = n is a, c, g, or t
  variation 774: note = n is a, c, g, or t
  variation 812: note = n is a, c, g, or t
  variation 835..836: note = n is a, c, g, or t
  variation 850: note = n is a, c, g, or t
  variation 854: note = n is a, c, g, or t
  variation 910: note = n is a, c, g, or t
  variation 963: note = n is a, c, g, or t
  variation 984: note = n is a, c, g, or t
  variation 1010: note = n is a, c, g, or t
  variation 1023..1026: note = n is a, c, g, or t
  variation 1036: note = n is a, c, g, or t
  variation 1058: note = n is a, c, g, or t
  variation 1066: note = n is a, c, g, or t
  variation 1068: note = n is a, c, g, or t
  variation 1078: note = n is a, c, g, or t
  variation 1080: note = n is a, c, g, or t
  variation 1082..1084: note = n is a, c, g, or t
  variation 1086: note = n is a, c, g, or t
  variation 1089..1090: note = n is a, c, g, or t
  variation 1092: note = n is a, c, g, or t
  variation 1095: note = n is a, c, g, or t
  variation 1098: note = n is a, c, g, or t
  source 1..1098: mol_type = other DNA
cnnnnnnngnnggngcgcagcagcagcaggaggagcccatggcctcggcgaggcgcagatcaagggcacgctcacccacggcggcaggtacgtgcagtacaacgtctacggcaacctcttcgaggtctccgccaagtacgtcccgcccatccgtcccgtcggccgcggcgcctgcggcatcatctgtgctgctatcaacgtgcagactcgcgaggaggtcgccatcaagaagatcggcaacgcgttcaacaaccagatcgacgccaagcgcactttgcgagaagtgaagctgcttcgacacatgaatcatgagaatgtgatttcaatcaaggacatcatacgcccgccaaggcgggagaattttaacgatgtttacatcgtttacgagctgatggacactgatcttcaccaccttcttagatcaaaccagccactcacagatgatcactgtcagtattttctctaccaagtgctcagaggattaaagtatgtgcactcggcgaatgtcttgcaccgagaccttaggccgagcaatctgttactgaatgccaaatgtgacctcaagattggggactttgggctggcgaggaccaccactgagactgacttcatgatggagtatgttgttacccggtggtatcgggcgccagagctccttcttaactgctcggagtacaccggagctattgatatgtggtcagtcggctgcatccttggtgagattgcaacaagagagcctctgtttcctggaaaagattacgttcatcagctgangctaattaccgagctgctaggctcaccagacgacaccngcctagggtttcttcggagcganntgcccgcagatatntcangtctcttcctcaataccccaagcaacaatttcgttcacggttcccaaatatgtccngtggcgccctggatttgcttgagaggatgcttgtatttgatccaaacaagagnattactgttgacgaggctctntgccatccctactggcatcccttcangagataaacgatnnnntgtctgcccngcgcctttcagctttgatttcnagcagccncntttaccgagnanannncnaannantcnttn

SEQ ID NO: 13 - protein, 387 aa, synthetic construct
  REGION 1..387: note = Synthetic: Bd21-3 (WT) BdMPK5
MRMEGGGAGPAAAAAAGGAHGLGEAQIKGTLTHGGRYVQYNVYGNLFEVSAKYVPPIRPVGRGACGIICAAINVQTREEVAIKKIGNAFDNQIDAKRTLREVKLLRHMNHENVISIKDIIRPPRRENFNDVYIVYELMDTDLHHLLRSNQPLTDDHCQYFLYQVLRGLKYVHSANVLHRDLRPSNLLLNAKCDLKIGDFGLARTTTETDFMMEYVVTRWYRAPELLLNCSEYTGAIDMWSVGCILGEIATREPLFPGKDYVHQLRLITELLGSPDDTSLGFLRSDNARRYVRSLPQYPKQQFRSRFPNMSSGAMDLLERMLVFDPNKRITVDEALCHPYLASLHEINDEPVCPAPFSFDFEQPSFTEEDIKELIWRESVKFNPEPIH

SEQ ID NO: 14 - protein, 387 aa, synthetic construct
  REGION 1..387: note = Synthetic: Chill1 BdMPK5
MRMEGGGAGPAAAAAAGGAHGLGEAQIKGTLTHGGRYVQYNVYGNLFEVSAKYVPPIRPVGRGACGIICAAINVQTREEVAIKKIGNAFNNQIDAKRTLREVKLLRHMNHENVISIKDIIRPPRRENFNDVYIVYELMDTDLHHLLRSNQPLTDDHCQYFLYQVLRGLKYVHSANVLHRDLRPSNLLLNAKCDLKIGDFGLARTTTETDFMMEYVVTRWYRAPELLLNCSEYTGAIDMWSVGCILGEIATREPLFPGKDYVHQLRLITELLGSPDDTSLGFLRSDNARRYVRSLPQYPKQQFRSRFPNMSSGAMDLLERMLVFDPNKRITVDEALCHPYLASLHEINDEPVCPAPFSFDFEQPSFTEEDIKELIWRESVKFNPEPIH

(19) United States
(12) Patent Application Publication
(10) Pub. No.: US 2026/0078398 A1
(43) Pub. Date: Mar. 19, 2026

(54) PLANTS GENETICALLY ENGINEERED FOR INCREASED WATER USE EFFICIENCY AND CARBON INTAKE AND METHODS FOR MAKING AND USING THEM

(71) Applicant: The Regents of the University of California, Oakland, CA (US)

(72) Inventors: Julian I. SCHROEDER, San Diego, CA (US); Yohei TAKAHASHI, San Diego, CA (US); Paulo Henrique DE OLIVEIRA CECILIATO, San Diego, CA (US)

(21) Appl. No.: 19/107,148

(22) PCT Filed: Sep. 1, 2023
(86) PCT No.: PCT/US2023/031849

§ 371 Date: Feb. 27, 2025

Related U.S. Application Data
(60) Provisional application No. 63/403,274, filed on Sep. 1, 2022.

(51) Int. Cl. C12N 15/82; C07K 14/415; C12N 9/12; C12N 15/113

(52) U.S. Cl. CPC C12N 15/8273; C07K 14/415; C12N 9/12; C12N 15/1137; C12Y 207/11001; C12Y 207/11024

(57) ABSTRACT

In alternative embodiments, provided are compositions and methods for manipulating the exchange of water and/or carbon dioxide (CO₂) through plant stomata comprising the step of modulating the expression of CO₂ sensor genes and the redundant MAP kinases MPK4/MPK12, and HT1 protein kinase, and their genes. In alternative embodiments, provided are dominant active HT1 mutants that cause a high CO2-insensitive constitutively open stomatal phenotype. In alternative embodiments, the invention provides plants, and plant tissues, having increased water use efficiency, and drought-resistant plants, plant tissues and cells; and methods for engineering of water transpiration and water use efficiency in plants, and engineering plants with increased water use efficiency and drought-resistant plants, plant tissues and cells. For plants grown in humid regions with sufficient water availability, down-regulation of the CO2 sensor proteins provides plants that show less stomatal closing in light of the atmospheric CO2 increase for enabling enhanced CO2 influx into leaves.

### RELATED APPLICATIONS

This application is a national phase application claiming benefit of priority under 35 U.S.C. § 371 to Patent Convention Treaty (PCT) International Application serial number PCT/US2023/031849, filed Sep. 1, 2023, now pending which claims the benefit of priority under 35 U.S.C. § 119 (e) of U.S. Provisional Patent Application Ser. No. (USSN) 63/403,274, Sep. 1, 2022. The aforementioned application are expressly incorporated herein by reference in their entirety and for all purposes. All publications, patents, patent applications cited herein are hereby expressly incorporated by reference for all purposes.

### STATEMENT AS TO FEDERALLY SPONSORED RESEARCH

“This invention was made with government support under ES010337 and GM060396 awarded by the National Institutes of Health and under MCB-1900567 awarded by the National Science Foundation. The government has certain rights in the invention”.

### REFERENCE TO ELECTRONIC SEQUENCE LISTING

The application contains a Sequence Listing which has been submitted electronically in .XML format and is hereby incorporated by reference in its entirety. Said .XML copy, created on Nov. 21, 2023, is named “0321.146838PCT.xml” and is 33,077 bytes in size. The sequence listing contained in this .XML file is part of the specification and is hereby incorporated by reference herein in its entirety.

### TECHNICAL FIELD

This invention generally relates to plant molecular and cellular biology. In alternative embodiments, provided are compositions and methods for manipulating the exchange of water and/or carbon dioxide (CO₂) through plant stomata comprising the step of modulating the expression of CO₂ sensor genes and the redundant MAP kinases MPK4/MPK12, and HT1 protein kinase, and their genes. In alternative embodiments, provided are dominant active HT1 mutants that cause a high CO₂-insensitive constitutively open stomatal phenotype. In alternative embodiments, the invention provides plants, plant tissues and cells, having increased water use efficiency, and drought-resistant plants, plant tissues and cells; and methods for engineering of water transpiration and water use efficiency in plants, and engineering plants/trees to maintain higher soil moisture, and engineering plants with increased water use efficiency and drought-resistant plants, plant tissues and cells.

### BACKGROUND

Carbon dioxide (CO2) influences the climate system as the major greenhouse gas. The continued rise in the atmospheric CO2 concentration critically influences plant growth, photosynthesis and plant water loss. Stomatal pores in leaves provide the conduits for CO2 influx for photosynthesis, while mediating over 95% of plant water loss via transpiration. Elevated CO2 triggers stomatal closing through protein phosphorylation mechanisms. However, the primary CO2 sensor that controls stomatal movements and plant water use efficiency remains unknown. The stomatal CO2 sensor has remained unknown despite many efforts over the past decades. Because of climate change, there is a need to understand how to manipulate and control stomatal movements and plant water use efficiency.

CO₂ influx into leaves is essential for plant carbon capture and growth. Plants lose 200 to greater than 500 water molecules via stomata for each carbon atom captured. CO2 regulation of the size (aperture) of stomatal gas exchange pores controls plant water loss by evapotranspiration to the outside air. Atmospheric CO₂ concentration is now 50% higher than pre-industrial times. Due to the high level of the plant “nutrient” CO₂ in the atmosphere, water use efficiency (WUE) of plants could be dramatically increased. Many crops and trees have weak stomatal CO₂ responses.

### SUMMARY

In alternative embodiments, provided are methods for:
increasing the water use efficiency of a plant leaf, a plant organ, a plant part or a plant;
increasing the rate of growth or biomass production in a plant leaf, a plant organ, a plant part or a plant;
enhancing the carbon dioxide (CO₂) sensitivity of a guard cell, a plant leaf, a plant organ, plant part or a plant;
down-regulating or decreasing carbon dioxide (CO₂) and/or water exchange in a plant leaf, a plant organ, a plant part or a plant;
decreasing the loss of water, while maintain uptake of carbon dioxide (CO₂) of a plant leaf, a plant organ, plant part or a plant; or
increasing the drought tolerance of a root cell, a plant leaf, a plant organ, a plant part or a plant; or
decreasing the heat resistance or tolerance (for example, under conditions of drought or increased atmospheric carbon dioxide) of a guard cell, a stomatal lineage stage-specific cell, a plant leaf, a plant organ, a plant part or a plant; comprising:
(a) in the guard cell, stomatal lineage stage-specific cell, plant leaf, plant organ, plant part or plant, increasing the expression and/or protein concentration or protein activity of:
a MAP kinase MPK4 and/or a MAP kinase MPK12;
(b) the method of (a), wherein the increasing of expression and/or protein concentration or protein activity of the MAP kinase MPK4 and/or the MAP kinase MPK12, is by:
   (1) providing a heterologous a MAP kinase MPK4, a MAP kinase MPK12, or a MAP kinase MPK4-, a MAP kinase MPK12-expressing nucleic acid (optionally a gene, cDNA or message) and expressing the gene, cDNA, message and/or protein in the guard cell, root cell, stomatal lineage stage-specific cell, plant leaf, plant organ, plant part or plant;
   (2) increasing of expression and/or protein concentration or protein activity of a homologous a MAP kinase MPK4-, a MAP kinase MPK12-expressing nucleic acid (optionally a gene, cDNA or message); or,
   (3) a combination of (1) and (2); or
(c) in a cell of the guard cell, stomatal lineage stage-specific cell, plant leaf, plant organ, plant part or plant, decreasing the expression and/or protein concentration or protein activity of:
   (1) a nucleic acid expressing a HT1 kinase; or
   (2) a HT1 kinase;
(b) the method of (a), wherein the decreasing of expression and/or protein concentration or protein activity of the HTI kinase, is by:
   (1) providing a heterologous antisense, iRNA, miRNA or artificial microRNA (miRNA) inhibitory to: the HT1 kinase-encoding nucleic acid (for example, to decrease or abrogate the expression or protein concentration or protein activity of a gene, cDNA or mRNA (message)), or any nucleic acid or compound inhibitory to the expression of the HT1 kinase; and,
expressing the inhibitory nucleic acid or compound, or the heterologous antisense, iRNA, miRNA or artificial microRNA (miRNA), in the guard cell, root cell, stomatal lineage stage-specific cell, plant leaf, plant organ, plant part or plant.

In alternative embodiments, provided are methods for:
up-regulating or increasing carbon dioxide (CO₂) and/or water exchange in a guard cell, a root cell, a stomatal lineage stage-specific cell, a plant leaf, a plant organ, a plant part or a plant;
decreasing the water use efficiency of a plant leaf, a plant organ, a plant part or a plant;
decreasing (desensitizing) the carbon dioxide (CO₂) sensitivity of a guard cell, a stomatal lineage stage-specific cell, a plant leaf, a plant organ, a plant part or a plant;
upregulating or increasing carbon dioxide (CO₂) and/or water exchange in a plant leaf, a plant organ, a plant part or a plant;
increasing the uptake of CO₂ in a plant leaf, a plant organ, a plant part or a plant;
decreasing drought tolerance in a plant leaf, a plant organ, a plant part or a plant; or
increasing the heat resistance or tolerance (for example, under conditions of drought or increased atmospheric carbon dioxide);
comprising:
(a) in a cell of the guard cell, stomatal lineage stage-specific cell, plant leaf, plant organ, plant part or plant, decreasing the expression and/or protein concentration or protein activity of:
   (1) a nucleic acid expressing a MAP kinase MPK4, a MAP kinase MPK12; or
   (2) a MAP kinase MPK4, a MAP kinase MPK12;
(b) the method of (a), wherein the decreasing of expression and/or protein concentration or protein activity of a MAP kinase MPK4, a MAP kinase MPK12, is by:
   (1) providing a heterologous antisense, iRNA, miRNA or artificial microRNA (miRNA) inhibitory to: the MAP kinase MPK4- and/or a MAP kinase MPK12-encoding nucleic acid (for example, to decrease or abrogate the expression or activity of a gene, cDNA or mRNA (message)), or any nucleic acid or compound inhibitory to the expression of the MAP kinase MPK4 and/or a MAP kinase MPK12; and,
   expressing the inhibitory nucleic acid or compound, or the heterologous antisense, iRNA, miRNA or artificial microRNA (miRNA), in the guard cell, root cell, stomatal lineage stage-specific cell, plant leaf, plant organ, plant part or plant;
   (2) decreasing of expression and/or activity of a homologous a MAP kinase MPK4-, a MAP kinase MPK12-encoding nucleic acid (optionally a gene, cDNA or mRNA (message)); or,
   (3) a combination of (1) and (2); or
(c) expressing or increasing the expression of a HT1-kinase-encoding gene, cDNA or mRNA or transcript, as described in any of the preceding claims, in the plant, guard cell, plant cell, plant leaf, plant organ or plant part.

In alternative embodiments, of methods as provided herein:
the MAP kinase MPK4 polypeptide comprises an amino acid sequence having between about 75% to 100% sequence identity, or 50%, 55%, 60%, 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or complete (100%) sequence identity, with SEQ ID NO:1,

(SEQ ID NO: 1)
MSAESCFGSSGDQSSSKGVATHGGSYVQYNVYGNLFEVSRKYVPPLRPIG
RGAYGIVCAATNSETGEEVAIKKIGNAFDNIIDAKRTLREIKLLKHMDHE
NVIAVKDIIKPPQRENFNDVYIVYELMDTDLHQIIRSNQPLTDDHCRFFL
YQLLRGLKYVHSANVLHRDLKPSNLLLNANCDLKLGDFGLARTKSETDFM
TEYVVTRWYRAPELLLNCSEYTAAIDIWSVGCILGETMTREPLFPGKDYV
HQLRLITELIGSPDDSSLGFLRSDNARRYVRQLPQYPRQNFAARFPNMSA
GAVDLLEKMLVFDPSRRITVDEALCHPYLAPLHDINEEPVCVRPFNFDFE
QPTLTEENIKELIYRETVKFNPQDSV;

the MAP kinase MPK12 polypeptide comprises an amino acid sequence having between about 75% to 100% sequence identity, or 50%, 55%, 60%, 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or complete (100%) sequence identity, with SEQ ID NO:2,

(SEQ ID NO: 2)
MSGESSSGSTEHCIKVVPTHGGRYVQYNVYGOLFEVSRKYVPPIRPIGRG
ACGIVCAAVNSVTGEKVAIKKIGNAFDNIIDAKRTLREIKLLRHMDHENV
ITIKDIVRPPQRDIFNDVYIVYELMDTDLQRILRSNQTLTSDQCRFLVYQ
LLRGLKYVHSANILHRDLRPSNVLLNSKNELKIGDFGLARTTSDTDFMTE
YVVTRWYRAPELLLNCSEYTAAIDIWSVGCILGEIMTGQPLFPGKDYVHQ
LRLITELVGSPDNSSLGFLRSDNARRYVRQLPRYPKQQFAARFPKMPTTA
IDLLERMLVFDPNRRISVDEALGHAYLSPHHDVAKEPVCSTPFSFDFEHP
SCTEEHIKELIYKESVKFNPDH;

the HT1 protein kinase polypeptide comprises an amino acid sequence having between about 75% to 100% sequence identity, or 50%, 55%, 60%, 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or complete (100%) sequence identity, with SEQ ID NO:3,

(SEQ ID NO: 3)
MSGLCFNPFRLRWSLRSKLPLEPSLPNLPCNPSSSKTNRYAEAETMEKKR
FDSMESWSMILESENVETWEASKGEREEWTADLSQLFIGNKFASGAHSRI
YRGIYKQRAVAVKMVRIPTHKEETRAKLEQQFKSEVALLSRLFHPNIVQF
IAACKKPPVYCIITEYMSQGNLRMYLNKKEPYSLSIETVLRLALDISRGM
EYLHSQGVIHRDLKSNNLLLNDEMRVKVADFGTSCLETQCREAKGNMGTY
RWMAPEMIKEKPYTRKVDVYSFGIVLWELTTALLPFQGMTPVQAAFAVAE
KNERPPLPASCQPALAHLIKRCWSENPSKRPDFSNIVAVLEKYDECVKEG
LPLTSHASLTKTKKAILDHLKGCVTSISSPFSSSSVPVNA;

and/or
the MAP kinase MPK4-, MAP kinase MPK12-, and/or HT1 protein kinase-expressing nucleic acid (for example, gene, cDNA or mRNA), is operably linked to a plant expressible promoter, an inducible promoter, a constitutive promoter, a root specific promoter, a stomatal lineage stage-specific cell specific promoter, a guard cell specific promoter, a drought-inducible promoter, a stress-inducible promoter or a guard cell active promoter.

In alternative embodiments, provided are methods for making a plant with enhanced water use efficiency (WUE), or drought-resistant plant, plant leaf, plant organ or plant part, comprising:
expressing or increasing the expression of a MAP kinase MPK4-, MAP kinase MPK12-encoding gene, cDNA or mRNA or transcript, as described in any of the preceding claims, in the plant, guard cell, plant cell, plant leaf, plant organ or plant part, or
decreasing the expression of an HT1 protein kinase protein-encoding gene or transcript in the plant, guard cell, plant cell, plant leaf, plant organ or plant part, by expressing a nucleic acid inhibitory to the expression of the HT1 protein kinase protein-expressing nucleic acid, gene, cDNA or mRNA or transcript as described in any one of the preceding claims, in the plant, guard cell, plant cell, plant leaf, plant organ, or plant part,
thereby regulating water uptake or water loss and increasing the WUE in the plant, plant cell, plant leaf, plant organ or plant part,
wherein optionally the increasing of the expression occurs in the plant guard cell or in a precursor cell of the plant guard cell.

In alternative embodiments, provided are methods for making a heat-resistant plant, plant leaf, plant organ, or plant part, comprising:
decreasing the expression of an HT1 protein kinase protein-encoding gene or transcript in the plant, guard cell, plant cell, plant leaf, plant organ or plant part, by expressing a nucleic acid inhibitory to the expression of the HT1 protein kinase protein-expressing nucleic acid, gene, cDNA or mRNA or transcript as described in any one of the preceding claims, in the plant, guard cell, plant cell, plant leaf, plant organ, or plant part, or
expressing or increasing the expression of a MAP kinase MPK4-, MAP kinase MPK12-encoding gene, cDNA or mRNA or transcript, as described in any of the preceding claims, in the plant, guard cell, plant cell, plant leaf, plant organ or plant part,
thereby making a heat-resistant plant, plant leaf, plant organ, or plant part
and optionally the decreasing of the expression occurs in the plant guard cell or in a precursor of the plant guard cell.

In alternative embodiments, of methods as provided herein, the plant is, or the guard cell, plant cell, plant part or plant organ, is isolated and/or derived from: (i) a dicotyledonous or monocotyledonous plant; (ii) wheat, oat, rye, barley, rice, sorghum, maize (corn), tobacco, a legume, a lupins, potato, sugar beet, pea, bean, soybean (soy), a cruciferous plant, a cauliflower, rape (or rapa or canola), cane (sugarcane), flax, cotton, palm, sugar beet, peanut, a tree, a poplar, a lupin, a silk cotton tree, desert willow, creosote bush, winterfat, balsa, ramie, kenaf, hemp, roselle, jute, or sisal abaca; or, (c) a species from the genera Anacardium, Arachis, Asparagus, Atropa, Avena, Brassica, Citrus, Citrullus, Capsicum, Carthamus, Cocos, Coffea, Cucumis, Cucurbita, Daucus, Elaeis, Fragaria, Glycine, Gossypium, Helianthus, Heterocallis, Hordeum, Hyoscyamus, Lactuca, Linum, Lolium, Lupinus, Lycopersicon, Malus, Man[iota]hot, Majorana, Medicago, Nicotiana, Olea, Oryza, Panieum, Pannisetum, Persea, Phaseolus, Pistachia, Pisum, Pyrus, Prunus, Raphanus, Ricinus, Secale, Senecio, Sinapis, Solanum, Sorghum, Theobromus, Trigonella, Triticum, Vicia, Vitis, Vigna or Zea.

In alternative embodiments, provided are a transgenic guard cell, plant, plant cell, plant tissue, plant seed or fruit, plant part or plant organ, comprising:
(a) a MAP kinase MPK4-, MAP kinase MPK12-, and/or HT1 protein kinase-expressing nucleic acid; or
wherein optionally the nucleic acid, gene or transcript is operably linked to a plant expressible promoter, an inducible promoter, a constitutive promoter, a root specific promoter, a stomatal lineage stage-specific cell specific promoter, a guard cell specific promoter, a drought-inducible promoter, a stress-inducible promoter or a guard cell active promoter;
and optionally the nucleic acid, gene or transcript is stably integrated into the genome of the guard cell, plant, plant cell, plant tissue, plant seed or fruit, plant part or plant organ, or is contained in an episomal vector in the guard cell, plant, plant cell, plant tissue, plant seed or fruit, plant part or plant organ.

In alternative embodiments, provided are a transgenic guard cell, plant, plant cell, plant tissue, plant seed or fruit, plant part or plant organ, comprising:
(a) (1) a heterologous nucleic acid that is inhibitory to a MAP kinase MPK4-, MAP kinase MPK12-, and/or HT1 protein kinase-expressing nucleic acid;
wherein optionally the inhibitory nucleic acid is operably linked to a plant expressible promoter, an inducible promoter, a constitutive promoter, a root specific promoter, a stomatal lineage stage-specific cell specific promoter, a guard cell specific promoter, a drought-inducible promoter, a stress-inducible promoter or a guard cell active promoter;
and optionally the inhibitory nucleic acid is stably integrated into the genome of the guard cell, plant, plant cell, plant tissue, plant seed or fruit, plant part or plant organ, or is contained in an episomal vector in the guard cell, plant, plant cell, plant tissue, plant seed or fruit, plant part or plant organ,
and optionally the inhibitory nucleic acid comprises an antisense RNA, siRNA, miRNA or an iRNA or an artificial micro RNA.

In alternative embodiments, provided are methods for genetically engineering variants or homologues of plant species as provided herein to have either reduced or enhanced expression or coding regions with reduced or enhanced activity of MPK4/MPK12 and/or HT1 orthologs in guard cells and/or leaves that show an altered CO₂ regulation of stomatal apertures; these genetically engineered variants or homologues can be used for marker assisted accelerated breeding of enhanced CO₂ sensor variants for improved water use efficiency or carbon capture.

The details of one or more exemplary embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference in their entireties for all purposes.

### DESCRIPTION OF DRAWINGS

The patent or application file contains at least one drawing executed in color. Copies of this patent or patent application publication with color drawing(s) will be provided by the Office upon request and payment of the necessary fee.

The drawings set forth herein are illustrative of exemplary embodiments provided herein and are not meant to limit the scope of the invention as encompassed by the claims.

FIG. 1A-D illustrate data showing that the CO₂ signaling Raf-like kinase CBC1 is activated by the HT1 protein kinase through phosphorylation:

FIG. 1A illustrates gel protein separation images showing that recombinant CBC1 and CBC2 proteins were incubated with or without HT1 proteins for 30 min with cold ATP, and in-gel kinase assays were performed;

FIG. 1B illustrates gel protein separation images showing that the kinase inactive CBC1-D253A and HT1-K113W protein isoforms were used for in vitro phosphorylation analyses with recombinant CBC1 and HT1 proteins as indicated;

FIG. 1C schematically illustrates a map of recombinant CBC1 proteins, which were incubated with or without HT1 and ATP, and CBC1 phosphorylation sites were identified by mass spectrometry, and HT1-dependent phosphorylation sites were identified. The underlined Ser-43 and Ser-45 as blue light-dependent phosphorylation sites;

FIG. 1D illustrates gel protein separation images showing data where CBC1-S43/S45A, S280A and T256/S280A proteins were used for in vitro phosphorylation assays, and the CBB gel (lower image) shows loading controls,
as discussed in detail in Example 1, below.

FIG. 2A-E illustrate data showing that MAP kinases MPK4 and MPK12 inhibit HT1-mediated CBC1 kinase phosphorylation in the presence of NaHCO₃ in vitro:

FIG. 2A illustrates gel protein separation images showing data where recombinant HT1 and CBC1 proteins were incubated with MPK4, MPK12 or the (pseudo)-kinase domain of GHR1 in the presence or absence of 20 mM NaHCO₃ for 30 min, and in vitro phosphorylation assays were performed;

FIG. 2B illustrates gel protein separation images showing data where MPK4, HT1 and CBC1 proteins were incubated with NaHCO₃ at the indicated concentrations for 30 min, and in vitro phosphorylation assays were performed;

FIG. 2C graphically illustrates CBC1 band intensities as shown in FIG. 2B, and they were measured using IMAGEJ™;

FIG. 2D illustrates gel protein separation images showing data where MPK4, HT1 and CBC1 proteins were incubated in reaction buffers adjusted at different pH (6.5 to 8.5) for 30 min, and in vitro phosphorylation assays were performed;

FIG. 2E graphically illustrates CBC1 band intensities and the density-ratios of ‘+NaHCO₃’ to ‘—NaHCO₃’ (=+NaCl controls) for each pH condition,
as discussed in detail in Example 1, below.

FIG. 3A-F illustrate data showing that the dominant HT1 mutations (HT1-R102K and A109V) disrupt HCO₃⁻ dependent downregulation of CBC1 protein kinase activity:

FIG. 3A-B illustrate data showing whole plant gas exchange analyses using ht1-G89R (FIG. 3A) and ht1-R173Q (FIG. 3B), ambient CO₂ concentrations are indicated by the top bars, n=7 experiments, error bars show±SEM;

FIG. 3C illustrates gel protein separation images showing data where recombinant HT1 (wild type, HT1-G89R or HT1-R173Q) and CBC1 proteins were incubated with MPK4 in the presence or absence of 20 mM NaHCO₃ or 20 mM NaCl (−controls) for 30 min, and in vitro phosphorylation assays were performed;

FIG. 3D illustrates gel protein separation images showing data where recombinant MPK12, CBC1 and HT1 (WT or R102K) proteins were incubated with or without 20 mM NaHCO₃ or 20 mM NaCl (−controls) for 30 min, and in vitro phosphorylation assays were performed. Histone was used as an artificial kinase substrate;

FIG. 3E illustrates gel protein separation images showing data where HT1 (WT or A109V) proteins were used for in vitro phosphorylation assays, proteins were incubated with or without 20 mM NaHCO₃ or 20 mM NaCl (−controls) for 30 min.;

FIG. 3F graphically illustrates data from studies where stomatal conductances were analyzed using intact plants of Arabidopsis (Col-0 (WT), cbc1 cbc2, ht1-A109V and cbc1 cbc2 ht1-A109V), CO₂ concentration changes were applied as indicated on top (p.p.m.),
as discussed in detail in Example 1, below.

FIG. 4A-J illustrate data showing that bicarbonate inactivates HT1 kinase by stabilizing HT1 interaction with MPK4/12:

FIG. 4A illustrates gel protein separation images showing data where recombinant CBC1, HT1 and MPK4 proteins are incubated with or without 20 mM NaHCO₃ for 30 min, and in vitro phosphorylation assays were performed;

FIG. 4B illustrates gel protein separation images showing data where CBC1 and HT1 proteins were incubated with or without 20 mM NaHCO₃ or 20 mM NaCl (“−” controls) in the presence or absence of MPK12 protein;

FIG. 4C illustrates gel protein separation images showing data where His-HT1 and GST-MPK4 or GST control proteins were used for in vitro pull-down assays with or without 20 mM NaHCO₃. NaHCO₃ or 20 mM NaCl (“−” controls) were supplemented in all buffers throughout the pull-down assay procedures including the washing step;

FIG. 4D illustrates gel protein separation images showing data where in vitro pull-down assays showed reversibility and were performed using the washing buffers supplemented with NaHCO₃ at the indicated concentrations (0, 2 or 20 mM);

FIG. 4E illustrates gel protein separation images showing data where in vitro pull-down assays were performed using recombinant HT1 (wild type, HT1-G89R and HT1-R173Q) proteins;

FIG. 4F illustrates gel protein separation images showing data where in vitro pull-down assays were performed using HT1-R102K isoform;

FIG. 4G illustrates gel protein separation images showing data where in vitro pull-down assays were performed using HT1-A109V isoform;

FIG. 4H illustrates gel protein separation images showing data where in vitro phosphorylation assays were performed after CBC1, HT1 and MPK12 or the MPK12-K70R kinase inactive isoform proteins were incubated with or without NaHCO₃ or NaCl (“−”) controls;

FIG. 4I graphically illustrates data from studies where stomatal conductances were analyzed in leaves of intact Arabidopsis plants [Col-0 (WT), mpk12, pGC1:MPK12-GFP mpk12 and pGC1:MPK12 (K70R)-GFP mpk12]; CO₂ concentration changes were applied as indicated on top (p.p.m.). n=6 experiments, error bars show ±SEM; and

FIG. 4J schematically illustrates an exemplary model of plant stomatal CO₂ sensor and signaling,
as discussed in detail in Example 1, below.

FIG. 5 illustrates data showing that Raf-like kinase CBC proteins are phosphorylated by HT1 protein kinase, and illustrates gel protein separation images showing data where in vitro phosphorylation analyses were performed using recombinant CBC1 and CBC2 proteins in the presence or absence of HT1, and histone was used as an artificial phosphorylation substrate of CBC1, as discussed in detail in Example 1, below.

FIG. 6 illustrates data showing an example of reversible interaction between MPK4 and HT1 in vitro; and illustrates gel protein separation images showing data where in vitro pull-down assays using recombinant GST-MPK4 and His-HT1 proteins were performed with or without 20 mM NaHCO₃, as discussed in detail in Example 1, below.

FIG. 7A-E illustrate data showing that a chill I mutant shows impaired stomatal response to changes in CO₂ but remains responsive to exogenous ABA:

FIG. 7A illustrates an image of 4 to 5 week old Brachypodium distachyon plants, as imaged following exposure to high CO₂ Pseudo-colored temperature scale is on the left; top image-infrared thermal image, bottom is image of photo of the same plants;

FIG. 7B-C graphically illustrate data where stomatal density (FIG. 7B) and stomatal index (FIG. 7C) were determined using leaf imprints created of the 4th true leaf of plants with 3 independent replicates plants per genotype with 4 images per plant, and circles represent counts per each image while diamonds are the average per each plant;

FIG. 7D graphically illustrate data where stomatal conductance was quantified using a gas exchange analyzer; and

FIG. 7E graphically illustrate data where stomatal conductance was recorded prior to and following addition of 2 μM ABA,
as discussed in further detail in Example 2, below.

FIG. 8A-B illustrate characterization of chill1 F1 backcross:

FIG. 8A illustrates an image of 5 to 6 week old backcrossed chill1 Brachypodium distachyon plants as analyzed (right) by infrared thermal imaging following 2 hour exposure to 1000 ppm CO₂ alongside parent line Bd 21-3 (WT) and the chill1 mutant, a pseudo-colored temperature scale is on the right; and

FIG. 8B graphically illustrate data where stomatal conductance response of the F1 cross of the parent line Bd21-3 with the chill1 mutant was analyzed using a gas exchange analyzer,
as discussed in further detail in Example 2, below.

FIG. 9A illustrates an image of 4 to 5 week old chill1×Bd 21-3 F2 backcrossed plants were exposed to 1000 ppm CO₂ for 2 hours then immediately imaged using the infrared thermal imaging camera, the left most plant is the Bd21-3 parent line, the drawn (blue) ellipse highlights a plant showing a chill1-like lower temperature, and a pseudo-colored temperature scale is on the right; and

FIG. 9A illustrates color images as taken at the same time for plant identification,
as discussed in further detail in Example 2, below.

FIG. 10A-B graphically illustrate QTLSEQR™ output for bulk-segregant analysis for M-Pool (FIG. 10A) and SM-Pool (FIG. 10B), respectively; and the genome-wide false discovery rate of 0.01 is indicated by the (red) line, as discussed in further detail in Example 2, below.

FIG. 11A-B illustrate candidate genes derived from BSA:

FIG. 11A illustrates a table showing variants in genes called as homozygous via Bulked Segregant analyses for the SM and M pools were compiled to create a list of called homozygous candidate mutations; and

FIG. 11B schematically illustrates the homozygous mutation within the 2nd exon of BdMPK5 (BdiBd21-3.3G0222500), and the exon sequence Bd 21-3 is GTCGCCATCAAGAAGATCGGCAACGCGTTCGACAACCAGATCGACGCCAA GCGCACTT (SEQ ID NO:4) as compared to the chill 1 sequence GTCGCCATCAAGAAGATCGGCAACGCGTTCAACAACCAGATCGACGCCAA GCGCACTT (SEQ ID NO:5) as shown by the circled nucleotides, as confirmed via sequencing, (see also SEQ ID NO:11, SEQ ID NO: 12, SEQ ID NO: 13 and SEQ ID NO: 14, as set forth in Example 2, below),
as discussed in further detail in Example 2, below.

FIG. 12A-D illustrates sodium azide line 0770 crosses:

FIG. 12A illustrates an image of 4- to 5-week old chill1, sequence indexed and confirmed sodium azide (NaN) line and the Bd21-3×chill1 F1 backcross were exposed to 1000 ppm CO₂ for 2 hours alongside the Bd21-3 parent (WT) control;

FIG. 12B illustrates color images as taken at the same time for identification of plants. Plants from left to right are labeled Bd21-3 (WT), chill1, NaN0770, and the F1 NaN0770 backcross;

FIG. 12C illustrates an image of sodium azide mutagenized NaN0770 line was crossed to chill1 and the F1 plants were used for infrared thermal imaging following exposure to high (1000 ppm) CO₂; and

FIG. 12D illustrates color images of the same plants, and plants from left to right are Bd 21-3, chill1, NaN0770 and the chill1×NaN0770 F1 cross. Pseudo-colored temperature scale is on the right in (FIG. 12A) and (FIG. 12C),
as discussed in further detail in Example 2, below.

FIG. 13A-B illustrate the design and Sequencing for isolation of chill1 CRISPR plants:

FIG. 13A illustrates guide RNA design used in generation of CRISPR plants in the Bd 21-3 parent background (highlighted in pink) and followed by the PAM sequence (highlighted in yellow); and

FIG. 13B illustrate sequences from plants generated using this gRNA, where the nucleotides were sequenced and two of the alleles generated are shown aligned to the parent sequence; alignment mis-matches between the CRISPR alleles and parent line are highlighted showing several small deletions near the PAM sequence:

Bd 21-3 (SEQ ID NO: 6)
(the underlined includes sequence of
the exon fragment shown in FIG. 11):
(SEQ ID NO: 6)
TGCTGCTATCAACGTGCAGACTCGCGAGGAGGTCGCCATCAAGAAGATCG
GCAACGCGTTCGACAACCAGATCGACG,
CRISPR #1 (SEQ ID NO: 7)
(where N can be any nucleotide),
(SEQ ID NO: 7)
TGCTGCTATCAACGTGCAGACTCCCCNAATAGACT
CRISPR #2 (SEQ ID NO: 8),
(SEQ ID NO: 8)
TGCTGCTATCAACGTGCAGACAGGAGGACACGTCGCCAACATCAGCAACG
CGTTCGACTTCCACATCCACA,

as discussed in further detail in Example 2, below.

FIG. 14A-D illustrate data showing that Bdmpk5 CRISPR alleles show strong CO₂ insensitivity but functional ABA responses:

FIG. 14A-B illustrate data from study where 5 to 6 week old plants were analyzed in time-resolved stomatal conductance analyses:

FIG. 14A graphically illustrates data showing that CRISPR alleles show strongly impaired stomatal responses to CO₂ shifts;

FIG. 14B graphically illustrates data showing that CRISPR leaves show functional ABA-induced stomatal closing, despite the large stomatal conductance; and

FIG. 14C-D graphically illustrate data showing that stomatal density (FIG. 14C) and stomatal index (FIG. 14D) were determined using leaf imprints created of the 4th true leaf with 3 replicate plants and genotype, and circles represent counts per each image while diamonds are the average per each plant, as discussed in further detail in Example 2, below.

FIG. 15A-B illustrate data showing that BdMPK5 functions together with Arabidopsis HT1 and CBC1 in high CO₂/bicarbonate-mediated down-regulation of CBC1 protein kinase phosphorylation and structural prediction of BdMPK5-HT1 complex:

FIG. 15A illustrates protein separation gels showing that, in contrast to wildtype BdMPK5 protein, the chill1 BdMPK5 (D90N) variant isoform does not mediate CO₂/HCO₃⁻-dependent downregulation of CBC1 kinase phosphorylation; and

FIG. 15B schematically illustrates an alphaFold2-predicted complex of BdMPK5 with HT1, with BdMPK5 is shown in red, while HT1 is shown in blue,
as discussed in further detail in Example 2, below.

FIG. 16A-D illustrate tiller harvesting and growth methods for F1 backcrossed plants enables generation of large number of F2 seeds:

FIG. 16A-B illustrate backcrossed F1 plants WT (Bd21-3)×chill1 (FIG. 16A) that were grown under short day (8 L: 16 D) until visible aerial root formation (FIG. 16B) could be observed; step 1 of tiller creation involves growing plants under short (8 D: 16 L) day until aerial root formation can be observed;

FIG. 16C illustrates tillers that were removed below the aerial root and placed into a nutrient containing solution; and

FIG. 16D illustrates aerial root growth prior to transfer to soil,
as discussed in further detail in Example 2, below.

FIG. 17A-B illustrate per base sequence quality of read one (FIG. 17A) and read two (FIG. 17B) for the raw sequencing data of the chill1-linked pool consisting of 57 chill1 BC1F2 individuals exhibiting a cooler leaf phenotype,
as discussed in further detail in Example 2, below.

FIG. 18 illustrates nucleic acid separation gels for genotyping of CRISPR plants to detect presence of Cas9: T2 CRISPR plants were genotyped to detect the presence of Cas9 using primers designed around the first 800 BP of the Cas9 sequence used in the transformation vector JD633 (Addgene); untransformed wildtype Bd21-3 plants were also used as controls as well as water as a negative control, bands indicate presence of Cas9 within T2 plants.

Like reference symbols in the various drawings indicate like elements.

### DETAILED DESCRIPTION

In alternative embodiments, provided are genetically modified plants and methods for manipulating the CO2 sensor which can be used for targeted engineering of plant water use efficiency and carbon intake in light of the continuing increase in the atmospheric CO2 concentration. In alternative embodiments, provided herein is a solution to a long-term question of how plants sense the continuing increase in the atmospheric CO2 concentration for regulation of the size (aperture) of stomatal gas exchange pores that allow CO₂ to enter plants from the air, while at the same time allowing over 90% of plant water loss by evapotranspiration to the outside air. In alternative embodiments, provided are methods and direct evidence for a powerful tool for improving plant water use efficiency of plants.

Alternative embodiments as provided herein are relevant for plant performance under water-limited and also in regions with water-replete conditions, given the continuing steep increase in the atmospheric CO2 concentration. For plants grown in arid regions, CO2-mediated reduction in stomatal pore apertures allows for reduced water consumption/loss by plants, while enabling robust CO2 intake into leaves for plant photosynthesis and growth. Alternatively, in humid regions with sufficient water availability, down-regulation of the CO2 sensor proteins provides plants that show less stomatal closing in light of the atmospheric CO2 increase for enabling enhanced CO2 influx into leaves, while also providing enhanced cooling of leaves due to enhanced evapotranspiration. The exchange of CO2 and water can be optimized in light of the increasing atmospheric CO2 concentration, as CO2 is a plant nutrient that is absorbed in exchange for water loss.

In alternative embodiments, provided are methods and transgenic plants that can be used for engineering of improved water use efficiency of plants via biochemical development, genome editing and over- and under-expression of the identified proteins and variants using promoters. The understanding into the structure and function of important sites allows selection of manipulated or naturally occurring allele variants in natural and wild populations for accelerated molecular marker assisted breeding of enhanced traits.

In alternative embodiments, provided are methods and transgenic plants that:
Result in dramatically enhanced water use efficiency of plants such as crop plants and trees;
Enhance drought tolerance of plants such as crop plants;
Reduced depletion of soil moisture by crops and trees;
Enhance growth under the present and continuing elevated CO₂
concentration of plants such as crop plants;
Enhance early monitoring of CO₂ elevation effects, drought and heat stress by plants such as crop plants and trees; and/or
Early mounting of stress resistance in plants such as crop plants and trees.

We have discovered the CO₂ sensor that controls CO₂ intake vs. water loss and can now improve water use efficiency (WUE). Two major applications of embodiments and technology as provided herein include:
(1) For plants grown in arid regions, CO₂-mediated reduction in stomatal pore apertures allows for reduced water consumption/loss by plants, while enabling robust CO₂ intake into leaves for plant photosynthesis and growth; and,
(2) Alternatively, in humid warm regions with sufficient water availability, down-regulation of the CO₂ sensor proteins provides plants that show less stomatal closing in light of the atmospheric CO₂ increase for enabling enhanced CO₂ influx into leaves, while also providing enhanced cooling of leaves due to enhanced evapotranspiration.

The exchange of CO₂ and water can be optimized in light of the continuing increase in the atmospheric CO₂ concentration, as CO₂ is a plant nutrient that is absorbed in exchange for water loss.

Agriculture uses approximately 70% of fresh water annually. The atmospheric CO₂ concentration is predicted to double during this century. This provides a unique opportunity to dramatically reduce the amount of water crops and trees use while improving carbon (CO₂) capture & avoiding soil and plant dessication. However, the CO₂ sensor that controls plant water loss has remained unknown. We have discovered the CO₂ sensor and its underlying mechanism. This enables greatly improving water use efficiency as the atmospheric CO₂ is rising.

This discovery further opens the door to marker-assisted accelerated breeding of enhanced CO₂ sensor variants for improved WUE and carbon capture. This breakthrough is relevant for developing plants, including crops, trees and other carbon sequesters in light of the continuing atmospheric CO₂ rise.

We have discovered that elevated CO2 triggers a reversible interaction of the redundant MAP kinases MPK4/MPK12 with the HT1 protein kinase. Bicarbonate anions are shown to be the active molecule causing MPK4/12-HT1 interaction. High bicarbonate-induced MPK4/12 interaction with HT1 in turn directly inhibits the kinase activity of HT1. The Raf-like protein kinases HT1 and CBC1 are strong negative regulators of high CO₂-induced stomatal closure. Upon CO₂/bicarbonate elevation, the MPK4/MPK12 kinases shut down the downstream CBC1 kinase via the MPK4/12 inhibition of HT1. This CO₂ down-regulation of the CBC1 kinase via the MPK-HT1 CO₂/bicarbonate-sensor is regulated by specific phosphorylation sites in CBC1 that are targeted by the HT1 kinase.

We further have unexpectedly found that the protein kinase activity of these MPKs is not required for the function of the CO₂ sensor. Transgenic MPK12 lines demonstrate that water transpiration and water use efficiency of these plants can be effectively manipulated. By forward genetic screening, we identify new dominant active HT1 mutants that cause a high CO2-insensitive constitutively open stomatal phenotype. These HT1 mutations abrogate the CO₂/bicarbonate-induced MPK4/12-HT1 interaction and HT1 inhibition and disrupt CO₂-mediated inhibition of CBC1.

Consistent with these findings epistasis analyses show that stomatal CO₂ signaling proceeds from the MPK4/12-HT1 CO2/bicarbonate sensor to CBC1 and CBC2. This advance defines specific amino acids in HT1 that are important for plant water loss manipulation and plant water use efficiency. These methods further provide an approach for defining these amino acids using direct biochemical methods, which are shown to affect plant water loss regulation in vivo.

Data described herein demonstrate that MPK4/12 (the redundant MAP kinases MPK4/MPK12) and HT1 together are the primary stomatal CO₂ sensor in plants that regulates plant water loss. The identification of the CO₂ sensor and signaling core that regulates stomatal conductance, the development of genetically modified plants and methods for manipulating the CO₂ sensor and findings with transgenic plants show that this advance can be used for targeted engineering of plant water use efficiency and carbon intake in light of the continuing increase in the atmospheric CO₂ concentration.

### Generating Transgenic Plants

### Plant (Expressible) Promoters

In alternative embodiments, promoters are used to drive the over-expression of a MAP kinase MPK4 and/or a MAP kinase MPK12 in a guard cell, stomatal lineage stage-specific cell, plant leaf, plant organ, plant part or plant, to increase the expression and/or protein concentration the kinase; or, promoters are used to drive the expression of nucleic acids inhibitory to a MAP kinase MPK4 and/or a MAP kinase MPK12 in a guard cell, stomatal lineage stage-specific cell, plant leaf, plant organ, plant part or plant, to decrease the expression and/or protein concentration the kinase.

In alternative embodiments, MAP kinase MPK4 and/or a MAP kinase MPK12-protein coding sequences or genes, or inhibitory sequences, used to practice methods as provided herein are operably linked to a plant expressible promoter, an inducible promoter, a constitutive promoter, a guard cell specific promoter, a drought-inducible promoter, a stress-inducible promoter or a guard cell active promoter. Promoters used to practice methods as provided herein include a strong promoter, particularly in plant guard cells, and in some embodiments is guard cell specific, for example, the promoters described in WO2008/134571.

In alternative embodiments, MAP kinase MPK4 and/or a MAP kinase MPK12-protein coding sequences or genes, or inhibitory sequences also can be operatively linked to any constitutive and/or plant specific, or plant cell specific promoter, for example, a cauliflower mosaic virus (CaMV) 35S promoter, a mannopine synthase (MAS) promoter, a 1′ or 2′ promoter derived from T-DNA of Agrobacterium tumefaciens, a figwort mosaic virus 34S promoter, an actin promoter, a rice actin promoter, a ubiquitin promoter, for example, a maize ubiquitin-1 promoter, and the like.

Examples of constitutive plant promoters which can be useful for expressing the protein-encoding sequences in accordance with methods as provided herein include: the cauliflower mosaic virus (CaMV) 35S promoter, which confers constitutive, high-level expression in most plant tissues (see, for example, Odell et al. (1985) Nature 313:810-812); the nopaline synthase promoter (An et al. (1988) Plant Physiol. 88:547-552); and the octopine synthase promoter (Fromm et al. (1989) Plant Cell 1:977-984).

A variety of plant gene promoters that regulate gene expression in response to environmental, hormonal, chemical, developmental signals, and in a tissue-active manner can be used for expression of a sequence in plants. Choice of a promoter is based largely on the phenotype of interest and is determined by such factors as tissue (for example, seed, fruit, root, pollen, vascular tissue, flower, carpel, etc.), inducibility (for example, in response to wounding, heat, cold, drought, light, pathogens, etc.), timing, developmental stage, and the like.

Numerous known promoters have been characterized and can be employed to promote expression of a polynucleotide used to practice methods as provided herein, for example, in a transgenic plant or cell of interest. For example, tissue specific promoters include: seed-specific promoters (such as the napin, phaseolin or DC3 promoter described in U.S. Pat. No. 5,773,697), fruit-specific promoters that are active during fruit ripening (such as the dru 1 promoter (U.S. Pat. No. 5,783,393), or the 2Al 1 promoter (for example, see U.S. Pat. No. 4,943,674) and the tomato polygalacturonase promoter (for example, see Bird et al. (1988) Plant Mol. Biol. 11:651-662), root-specific promoters, such as those disclosed in U.S. Pat. Nos. 5,618,988, 5,837,848 and 5,905,186, pollen-active promoters such as PTA29, PTA26 and PTA1 3 (for example, see U.S. Pat. No. 5,792,929), promoters active in vascular tissue (for example, see Ringli and Keller (1998) Plant Mol. Biol. 37:977-988), flower-specific (for example, see Kaiser et al. (1995) Plant Mol. Biol. 28:231-243), pollen (for example, see Baerson et al. (1994) Plant Mol. Biol. 26:1947-1959), carpels (for example, see OhI et al. (1990) Plant Cell 2: pollen and ovules (for example, see Baerson et al. (1993) Plant Mol. Biol. 22:255-267), auxin-inducible promoters (such as that described in van der Kop et al. (1999) Plant Mol. Biol. 39:979-990 or Baumann et al., (1999) Plant Cell 11:323-334), cytokinin-inducible promoter (for example, see Guevara-Garcia (1998) Plant Mol. Biol. 38:743-753), promoters responsive to gibberellin (for example, see Shi et al. (1998) Plant Mol. Biol. 38:1053-1060, Willmott et al. (1998) Plant Molec. Biol. 38:817-825) and the like.

Additional promoters that can be used to practice methods as provided herein are those that elicit expression in response to heat (for example, see Ainley et al. (1993) Plant Mol. Biol. 22:13-23), light (for example, the pea rbcS-3A promoter, Kuhlemeier et al. (1989) Plant Cell 1:471-478, and the maize rbcS promoter, Schaffher and Sheen (1991) Plant Cell 3:997-1012); wounding (for example, wunl, Siebertz et al. (1989) Plant Cell 1:961-968); pathogens (such as the PR-I promoter described in Buchel et al. (1999) Plant Mol. Biol. 40:387-396, and the PDF 1.2 promoter described in Manners et al. (1998) Plant Mol. Biol. 38:1071-1080), and chemicals such as methyl jasmonate or salicylic acid (for example, see Gatz (1997) Annu. Rev. Plant Physiol. Plant Mol. Biol. 48:89-108). In addition, the timing of the expression can be controlled by using promoters such as those acting at senescence (for example, see Gan and Amasino (1995) Science 270:1986-1988); or late seed development (for example, see Odell et al. (1994) Plant Physiol. 106:447-458).

In alternative embodiments, tissue-specific and/or developmental stage-specific promoters are used, for example, promoter that can promote transcription only within a certain time frame of developmental stage within that tissue. See, for example, Blazquez (1998) Plant Cell 10:791-800, characterizing the Arabidopsis LEAFY gene promoter. See also Cardon (1997) Plant J 12:367-77, describing the transcription factor SPL3, which recognizes a conserved sequence motif in the promoter region of the A. thaliana floral meristem identity gene API; and Mandel (1995) Plant Molecular Biology, Vol. 29, pp 995-1004, describing the meristem promoter eIF4. Tissue specific promoters which are active throughout the life cycle of a particular tissue can be used.

In one aspect, MAP kinase MPK4 and/or a MAP kinase MPK12-protein coding sequences or genes, or inhibitory sequences, used to practice methods as provided herein are operably linked to a promoter active primarily only in cotton fiber cells.

Root-specific promoters may also be used to express MAP kinase MPK4 and/or a MAP kinase MPK12-encoding nucleic acids used to practice methods as provided herein. Examples of root-specific promoters include the promoter from the alcohol dehydrogenase gene (DeLisle (1990) Int. Rev. Cytol. 123:39-60). Other promoters that can be used to express M3K-encoding nucleic acids used in methods as provided herein include, for example, ovule-specific, embryo-specific, endosperm-specific, integument-specific, seed coat-specific promoters, or some combination thereof; a leaf-specific promoter (see, for example, Busk (1997) Plant J. 11:1285 1295, describing a leaf-specific promoter in maize); the ORF 13 promoter from Agrobacterium rhizogenes (which exhibits high activity in roots, see, for example, Hansen (1997) supra); a maize pollen specific promoter (see, for example, Guerrero (1990) Mol. Gen. Genet. 224:161 168); a tomato promoter active during fruit ripening, senescence and abscission of leaves and, to a lesser extent, of flowers can be used (see, for example, Blume (1997) Plant J. 12:731 746); a pistil-specific promoter from the potato SK2 gene (see, for example, Ficker (1997) Plant Mol. Biol. 35:425 431); the Blec4 gene from pea, which is active in epidermal tissue of vegetative and floral shoot apices of transgenic alfalfa making it a useful tool to target the expression of foreign genes to the epidermal layer of actively growing shoots or fibers; the ovule-specific BELI gene (see, for example, Reiser (1995) Cell 83:735-742, GenBank No. U39944); and/or, the promoter in Klee, U.S. Pat. No. 5,589,583, describing a plant promoter region is capable of conferring high levels of transcription in meristematic tissue and/or rapidly dividing cells.

In alternative embodiments, plant promoters used in methods as provided herein can be inducible upon exposure to plant hormones, such as auxins; these promoters can be used to express MAP kinase MPK4 and/or a MAP kinase MPK12 nucleic acids used in methods as provided herein. For example, exemplary methods can use the auxin-response elements El promoter fragment (AuxREs) in the soybean {Glycine max L.) (Liu (1997) Plant Physiol. 115:397-407); the auxin-responsive Arabidopsis GST6 promoter (also responsive to salicylic acid and hydrogen peroxide) (Chen (1996) Plant J. 10:955-966); the auxin-inducible parC promoter from tobacco (Sakai (1996) 37:906-913); a plant biotin response element (Streit (1997) Mol. Plant Microbe Interact. 10:933-937); and, the promoter responsive to the stress hormone abscisic acid (Sheen (1996) Science 274:1900-1902).

In alternative embodiments, MAP kinase MPK4 and/or a MAP kinase MPK12-encoding nucleic acids used in methods as provided herein can also be operably linked to plant promoters which are inducible upon exposure to chemicals reagents which can be applied to the plant, such as herbicides or antibiotics. For example, the maize In2-2 promoter, activated by benzenesulfonamide herbicide safeners, can be used (De Veylder (1997) Plant Cell Physiol. 38:568-577); application of different herbicide safeners induces distinct gene expression patterns, including expression in the root, hydathodes, and the shoot apical meristem. Coding sequence can be under the control of, for example, a tetracycline-inducible promoter, for example, as described with transgenic tobacco plants containing the Avena sativa L. (oat) arginine decarboxylase gene (Masgrau (1997) Plant J. 11:465-473); or, a salicylic acid-responsive element (Stange (1997) Plant J. 11:1315-1324). Using chemically, for example, hormone- or pesticide-induced promoters, i.e., promoter responsive to a chemical which can be applied to the transgenic plant in the field, expression of a polypeptide can be induced at a particular stage of development of the plant.

In alternative embodiments, provided are transgenic plants containing an inducible gene encoding for polypeptides used to practice methods as provided herein whose host range is limited to target plant species, such as corn, rice, barley, wheat, potato or other crops, inducible at any stage of development of the crop.

In alternative embodiments, a tissue-specific plant promoter may drive expression of operably linked sequences in tissues other than the target tissue. In alternative embodiments, a tissue-specific promoter that drives expression preferentially in the target tissue or cell type, but may also lead to some expression in other tissues as well, is used.

In alternative embodiments, proper polypeptide expression may require polyadenylation region at the 3′-end of the coding region. The polyadenylation region can be derived from the natural gene, from a variety of other plant (or animal or other) genes, or from genes in the Agrobacterial T-DNA.

### Engineering Plants to Express MPK4 or MAP Kinase Nucleic Acids

In alternative embodiments, provided are transgenic plants, plant parts, plant organs or tissue, and seeds comprising a nucleic acid that encodes an MAP kinase MPK4 and/or a MAP kinase MPK12 family enzyme, and expression cassettes or vectors, or a transfected or transformed cell, or transgenic plant comprising or having contained therein an MAP kinase MPK4 and/or a MAP kinase MPK12 family enzyme-encoding nucleic acid, or inhibitory nucleic acids. Also provided are plant products, for example, seeds, leaves, extracts and the like, comprising an MAP kinase MPK4 and/or a MAP kinase MPK12 family enzyme-encoding nucleic acid.

In alternative embodiments, the transgenic plant can be dicotyledonous (a dicot) or monocotyledonous (a monocot). Also provided are methods of making and using these transgenic plants and seeds. The engineered transgenic plant or plant cell expressing, or over-expressing, an MAP kinase MPK4 and/or a MAP kinase MPK12 family polypeptide may be constructed in accordance with any method known in the art. See, for example, U.S. Pat. No. 6,309,872; or 8,710,207, which describes expression control sequences (ECS) operable in monocots and/or dicots; or U.S. Pat. No. 9,121,027, which describes methods for transforming monocotyledonous plants; or U.S. Pat. No. 11,330,776, which rapid and efficient transformation of plants; or U.S. patent publication US2020/0270621 A1, which describes vectors that can be used to express nucleic acids in monocots; or U.S. patent publication US2022/0073890A1, which describes genome modification of a target sequence in the genome of a cell, using a novel Cas endonuclease; or US 2021/0054390 A1, which describes plant genome editing.

Nucleic acids and expression constructs used to practice methods as provided herein can be introduced into a plant cell by any means. For example, nucleic acids or expression constructs can be introduced into the genome of a desired plant host, or, the nucleic acids or expression constructs can be episomes. Introduction into the genome of a desired plant can be such that the host's MAP kinase MPK4 and/or a MAP kinase MPK12 production is regulated by endogenous transcriptional or translational control elements, or by a heterologous promoter, for example, a promoter used to drive expression of a MAP kinase MPK4 and/or a MAP kinase MPK12 family enzyme-expressing nucleic acid.

Also provided are engineered plants where insertion of gene sequence into the genome by, for example, homologous recombination, inserts a MAP kinase MPK4 and/or a MAP kinase MPK12 family polypeptide-encoding nucleic acid sequence.

The nucleic acids practice methods as provided herein can be expressed in or inserted in any plant, plant part, plant cell or seed.

Transgenic plants or a plant or plant cell comprising a nucleic acid used to practice methods as provided herein (for example, a transfected, infected or transformed cell) can be dicotyledonous or monocotyledonous. Examples of monocots comprising a MAP kinase MPK4 and/or a MAP kinase MPK12 family enzyme-expressing nucleic acid, for example, as monocot transgenic plants as provided herein, are grasses, such as meadow grass (blue grass, Pod), forage grass such as festuca, lolium, temperate grass, such as Agrostis, and cereals, for example, wheat, oats, rye, barley, rice, sorghum, and maize (corn). Examples of dicots comprising a MAP kinase MPK4 and/or a MAP kinase MPK12 family enzyme-expressing nucleic acid, for example, as dicot transgenic plants as provided herein, are tobacco, legumes, such as lupins, potato, sugar beet, pea, bean and soybean, and cruciferous plants (family Brassicaceae), such as cauliflower, rape seed, and the closely related model organism Arabidopsis thaliana. Thus, plant or plant cell comprising a MAP kinase MPK4 and/or a MAP kinase MPK12 family enzyme-expressing nucleic acid, including the transgenic plants and seeds as provided herein, include a broad range of plants, including, but not limited to, species from the genera Anacardium, Arachis, Asparagus, Atropa, Avena, Brassica, Citrus, Citrullus, Capsicum, Carthamus, Cocos, Cojfea, Cucumis, Cucurbita, Daucus, Elaeis, Fragaria, Glycine, Gossypium, Helianthus, Heterocallis, Hordeum, Hyoscyamus, Lactuca, Linum, Lolium, Lupinus, Lycopersicon, Malus, Manihot, Majorana, Medicago, Nicotiana, Olea, Oryza, Panieum, Pannisetum, Persea, Phaseolus, Pistachia, Pisum, Pyrus, Prunus, Raphanus, Ricinus, Secale, Senecio, Sinapis, Solarium, Sorghum, Theobromus, Trigonella, Triticum, Vicia, Vitis, Vigna, and Zea.

The nucleic acids used to practice methods as provided herein can be expressed in or inserted in any plant cell, organ, seed or tissue, including differentiated and undifferentiated tissues or plants, including but not limited to roots, stems, shoots, cotyledons, epicotyl, hypocotyl, leaves, pollen, seeds, tumor tissue and various forms of cells in culture such as single cells, protoplast, embryos, and callus tissue. The plant tissue may be in plants or in organ, tissue or cell culture.

### Transgenic Plants

In alternative embodiments, provided are transgenic plants, plant cells, organs, seeds or tissues, comprising and expressing the nucleic acids used to practice methods as provided herein, including MAP kinase MPK4 and/or a MAP kinase MPK12 family-expressing genes; for example, provided are plants, for example, transgenic plants, plant cells, organs, seeds or tissues that show improved growth under limiting water conditions; thus, provided are drought-tolerant plants, plant cells, organs, seeds or tissues (for example, crops).

A transgenic plant as provided herein can also include the machinery necessary for increasing the expression or activity of MAP kinase MPK4 and/or a MAP kinase MPK12 family polypeptides encoded by an endogenous gene, for example, by altering the phosphorylation state of the polypeptide to maintain it in an activated state.

Transgenic plants (or plant cells, or plant explants, or plant tissues) expressing or over-expressing MAP kinase MPK4 and/or a MAP kinase MPK12 family polypeptides, or inhibitory nucleic acids, can be produced by a variety of well-established techniques as described above.

Following construction of a vector, most typically an expression cassette, including a polynucleotide, for example, encoding a transcription factor or transcription factor homolog, standard techniques can be used to introduce the M3K δ B3 family polynucleotide into a plant, a plant cell, a plant explant or a plant tissue of interest. In one aspect the plant cell, explant or tissue can be regenerated to produce a transgenic plant. The plant can be any higher plant, including gymnosperms, monocotyledonous and dicotyledonous plants. Suitable protocols are available for Leguminosae (alfalfa, soybean, clover, etc.), Umbelliferae (carrot, celery, parsnip), Cruciferae (cabbage, radish, rapeseed, broccoli, etc.), Curcurbitaceae (melons and cucumber), Gramineae (wheat, corn, rice, barley, millet, etc.), Solanaceae (potato, tomato, tobacco, peppers, etc.), and various other crops. See protocols described in Ammirato et al., eds., (1984) Handbook of Plant Cell Culture—Crop Species, Macmillan Publ. Co., New York, N. Y.; Shimamoto et al. (1989) Nature 338:274-276; Fromm et al. (1990) Bio/Technol. 8:833-839; and Vasil et al. (1990) Bio/Technol. 8:429-434.

Transformation and regeneration of both monocotyledonous and dicotyledonous plant cells is now routine, and the selection of the most appropriate transformation technique will be determined by the practitioner. The choice of method will vary with the type of plant to be transformed; those skilled in the art will recognize the suitability of particular methods for given plant types. Suitable methods can include, but are not limited to: electroporation of plant protoplasts; liposome-mediated transformation; polyethylene glycol (PEG) mediated transformation; transformation using viruses; micro-injection of plant cells; micro-projectile bombardment of plant cells; vacuum infiltration; and

In alternative embodiments, an Agrobacterium tumefaciens mediated transformation is used. Transformation means introducing a nucleotide sequence into a plant in a manner to cause stable or transient expression of the sequence.

Successful examples of the modification of plant characteristics by transformation with cloned sequences which serve to illustrate the current knowledge in this field of technology, and include for example: U.S. Pat. Nos. 5,571,706; 5,677,175; 5,510,471; 5,750,386; 5,597,945; 5,589,615; 5,750,871; 5,268,526; 5,780,708; 5,538,880; 5,773,269; 5,736,369 and 5,619,042.

In alternative embodiments, following transformation, plants are selected using a dominant selectable marker incorporated into the transformation vector. Such a marker can confer antibiotic or herbicide resistance on the transformed plants, and selection of transformants can be accomplished by exposing the plants to appropriate concentrations of the antibiotic or herbicide.

In alternative embodiments, after transformed plants are selected and grown to maturity, those plants showing a modified trait (for example, expression or overexpression of MAP kinase MPK4 and/or a MAP kinase MPK12 family enzyme) are identified. The modified trait can be any of those traits described above. In alternative embodiments, to confirm that the modified trait is due to changes in expression levels or activity of the transgenic polypeptide or polynucleotide can be determined by analyzing mRNA expression using Northern blots, RT-PCR or microarrays, or protein expression using immunoblots or Western blots or gel shift assays.

Nucleic acids and expression constructs can be introduced into a plant cell by any means. For example, nucleic acids or expression constructs can be introduced into the genome of a desired plant host, or, the nucleic acids or expression constructs can be episomes. Introduction into the genome of a desired plant can be such that the host's CO2 sensor production is regulated by endogenous transcriptional or translational control elements.

In alternative embodiments, provided are “knockout plants” where insertion of a gene sequence by, for example, homologous recombination, can result in MAP kinase MPK4 and/or a MAP kinase MPK12 over-expression. Means to generate “knockout” plants are well-known in the art, see, for example, Strepp (1998) Proc Natl. Acad. Sci. USA 95:4368-4373; Miao (1995) Plant J 7:359-365. See discussion on transgenic plants, below.

In alternative embodiments, making transgenic plants or seeds comprises incorporating sequences used to practice methods as provided herein and, in one aspect (optionally), marker genes into a target expression construct (for example, a plasmid), along with positioning of the promoter and the terminator sequences. This can involve transferring the modified gene into the plant through a suitable method. For example, a construct may be introduced directly into the genomic DNA of the plant cell using techniques such as electroporation and microinjection of plant cell protoplasts, or the constructs can be introduced directly to plant tissue using ballistic methods, such as DNA particle bombardment. For example, see, for example, Christou (1997) Plant Mol. Biol. 35:197-203; Pawlowski (1996) Mol. Biotechnol. 6:17-30; Klein (1987) Nature 327:70-73; Takumi (1997) Genes Genet. Syst. 72:63-69, discussing use of particle bombardment to introduce transgenes into wheat; and Adam (1997) supra, for use of particle bombardment to introduce YACs into plant cells. For example, Rinehart (1997) supra, used particle bombardment to generate transgenic cotton plants. Apparatus for accelerating particles is described U.S. Pat. No. 5,015,580; and, the commercially available BioRad (Biolistics) PDS-2000 particle acceleration instrument; see also, John, U.S. Pat. No. 5,608,148; and Ellis, U.S. Pat. No. 5,681,730, describing particle-mediated transformation of gymnosperms.

In alternative embodiments, protoplasts can be immobilized and injected with a nucleic acid, for example, an expression construct. Although plant regeneration from protoplasts is not easy with cereals, plant regeneration is possible in legumes using somatic embryogenesis from protoplast derived callus. Organized tissues can be transformed with naked DNA using gene gun technique, where DNA is coated on tungsten microprojectiles, shot 1/100th the size of cells, which carry the DNA deep into cells and organelles. Transformed tissue is then induced to regenerate, usually by somatic embryogenesis. This technique has been successful in several cereal species including maize and rice.

In alternative embodiments, a third step can involve selection and regeneration of whole plants capable of transmitting the incorporated target gene to the next generation. Such regeneration techniques rely on manipulation of certain phytohormones in a tissue culture growth medium, typically relying on a biocide and/or herbicide marker that has been introduced together with the desired nucleotide sequences. Plant regeneration from cultured protoplasts is described in Evans et al., Protoplasts Isolation and Culture, Handbook of Plant Cell Culture, pp. 124-176, MacMillilan Publishing Company, New York, 1983; and Binding, Regeneration of Plants, Plant Protoplasts, pp. 21-73, CRC Press, Boca Raton, 1985. Regeneration can also be obtained from plant callus, explants, organs, or parts thereof. Such regeneration techniques are described generally in Klee (1987) Ann. Rev. of Plant Phys. 38:467-486. To obtain whole plants from transgenic tissues such as immature embryos, they can be grown under controlled environmental conditions in a series of media containing nutrients and hormones, a process known as tissue culture. Once whole plants are generated and produce seed, evaluation of the progeny begins.

In alternative embodiments, after the expression cassette is stably incorporated in transgenic plants, it can be introduced into other plants by sexual crossing. Any of a number of standard breeding techniques can be used, depending upon the species to be crossed. Since transgenic expression of a MAP kinase MPK4 and/or a MAP kinase MPK12 family enzyme-expressing nucleic acid leads to phenotypic changes, plants comprising the recombinant nucleic acids comprising a MAP kinase MPK4 and/or a MAP kinase MPK12 family enzyme-expressing nucleic acid can be sexually crossed with a second plant to obtain a final product. Thus, a seed containing a MAP kinase MPK4 and/or a MAP kinase MPK12 family enzyme-expressing nucleic acid can be derived from a cross between two transgenic plants as provided herein, or a cross between a plant comprising a MAP kinase MPK4 and/or a MAP kinase MPK12 family enzyme-expressing nucleic acid and another plant. The desired effects (for example, over-expression of a MAP kinase MPK4 and/or a MAP kinase MPK12 family enzyme) can be enhanced when both parental plants express the polypeptides, for example, a MAP kinase MPK4 and/or a MAP kinase MPK12 family enzyme. The desired effects can be passed to future plant generations by standard propagation means.

### CRISPR in Plants

In alternative embodiments, provided are methods that comprise decreasing the expression and/or protein concentration or protein activity of: a nucleic acid expressing a MAP kinase MPK4, a MAP kinase MPK12; or a MAP kinase MPK4, a MAP kinase MPK12, in a cell of the guard cell, stomatal lineage stage-specific cell, plant leaf, plant organ, plant part or plant; and in alternative embodiments a CRISPR system such as CRISPR-Cas9 system is used, see also for example, discussion in Example 2.

Delivery of a MAP kinase MPK4, a MAP kinase MPK12; or a MAP kinase MPK4, a MAP kinase MPK12-inhibitory exogenous nucleic acids, Cas9, sgRNA, and associated complexes into cells such as T cells can occur using viral and non-viral systems: for example, electroporation of DNA, RNA, or ribo-nucleocomplexes; chemical transfection techniques utilizing lipids and peptides (particularly to introduce sgRNAs in complex with Cas9 into cells); nanoparticle-based delivery for transfection, and the like. Some categories of cells are more difficult to transfect, including stem cells, neurons, and hematopoietic cells, and these require more efficient delivery systems, such as those based on lentivirus (LVs), adenovirus (AdV), and adeno-associated virus (AAV).

Variants of CRISPR-Cas9 an be used to allow gene activation or genome editing with an external trigger such as light or small molecules: including photoactivatable CRISPR systems developed by fusing light-responsive protein partners with an activator domain and a dCas9 for gene activation, or by fusing similar light-responsive domains with two constructs of split-Cas9, or by incorporating caged unnatural amino acids into Cas9, or by modifying the guide RNAs with photocleavable complements for genome editing.

In alternative embodiments, “dead” versions of Cas9 (dCas9) are used to eliminate CRISPR's DNA-cutting ability while preserving its ability to target desirable sequences. Various regulatory factors can be added to dCas9s, enabling turning any gene on or off or adjust its level of activity. Like RNAi, CRISPR interference (CRISPRi) can turn off genes in a reversible fashion by targeting, but not cutting a site. The targeted site is methylated, epigenetically modifying the gene. This modification inhibits transcription. These precisely placed modifications may then be used to regulate the effects on gene expression (for example, HMGB2) and DNA dynamics after the inhibition of certain genome sequences within DNA.

In alternative embodiments, CRISPR-Cas13 fused to deaminases is used to direct mRNA editing; for example, Cas7-11, is better suited for therapeutic RNA editing than Cas13, and enables sufficiently targeted cuts.

In alternative embodiments, any CRISPR system can be used to practice methods as provided herein, for example, as described in US 2022 0387560 A1, which describes methods of treating and/or correcting ocular disease in vivo using an Adeno-associated virus (AAV) system, where the AAV system employs a nucleic acid encoding a CRISPR-Cas9 system for targeted gene disruption or correction; or US 2022 0389398 A1 that describes using engineered CRISPR/Cas effector enzymes, such as Cas13 (Cas13d, Cas13e, or Cas13f) that maintain guide-sequence-specific endonuclease activity and lack guide-sequence-independent collateral endonuclease activity; or US 2023 0029506 A1, which describes therapeutic applications of the crispr-cas systems and compositions for genome editing; or, US 2020 0340012 A1, which describes a modular CRISPR-Cas9 architecture that allows better delivery, specificity and selectivity of gene editing; or U.S. Pat. No. 8,771,945, which describes CRISPR-Cas systems and methods for altering expression of gene products; or WO 2023 283420 A2 which describes therapeutic gene silencing with crispr-cas13.

### Products of Manufacture and Kits

Provided are products of manufacture and kits for practicing methods as provided herein; and optionally, products of manufacture and kits can further comprise instructions for practicing methods as provided herein.

Any of the above aspects and embodiments can be combined with any other aspect or embodiment as disclosed here in the Summary, Figures and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About (use of the term “about”) can be understood as within 20%, 19%, 18%, 17%, 16%, 15%, 14%, 13%, 12% 11%, 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless specifically stated or obvious from context, as used herein, the terms “substantially all”, “substantially most of”, “substantially all of” or “majority of” encompass at least about 75%, 80%, 85%, 90%, 91%, 92%, 93%, 94%, 95%, 96%, 97%, 98%, 99% or 99.5%, or more of a referenced amount of a composition.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

The invention will be further described with reference to the examples described herein; however, it is to be understood that the invention is not limited to such examples.

### EXAMPLES

Unless stated otherwise in the Examples, all recombinant DNA techniques are carried out according to standard protocols, for example, as described in Sambrook et al. (2012) Molecular Cloning: A Laboratory Manual, 4th Edition, Cold Spring Harbor Laboratory Press, NY and in Volumes 1 and 2 of Ausubel et al. (1994) Current Protocols in Molecular Biology, Current Protocols, USA. Standard materials and methods for plant molecular work are described in Plant Molecular Biology Labfax (1993) by R. D. D. Croy, jointly published by BIOS Scientific Publications Ltd (UK) and Blackwell Scientific Publications, UK. Other references for standard molecular biology techniques include Sambrook and Russell (2001) Molecular Cloning: A Laboratory Manual, Third Edition, Cold Spring Harbor Laboratory Press, NY, Volumes I and II of Brown (1998) Molecular Biology LabFax, Second Edition, Academic Press (UK). Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR-Basics: From Background to Bench, First Edition, Springer Verlag, Germany.

### Example 1: Guard Cell CO₂/Bicarbonate Sensing is Mediated by Two Interacting Protein Kinases

This example demonstrates that methods and genetically engineered plants as provided herein are effective and can be used to engineer water transpiration and water use efficiency in plants, and engineered plants, plant tissues and cells as provided herein have increased water use efficiency and are drought-resistant.

The continuing rise in the atmospheric carbon dioxide (CO₂) concentration causes stomatal closing, thus critically affecting transpirational water loss, photosynthesis and plant growth. However, the primary CO₂ sensor remains unknown. Here, we show that elevated CO₂ triggers interaction of the MAP kinases MPK4/MPK12 with the HT1 protein-kinase, thus inhibiting HT1 activity. At low CO₂, HT1 phosphorylates and thus activates the downstream negatively-regulating CBC1 kinase. In a genetic screen we identify dominant active HT1 mutants that cause insensitivity to elevated CO₂. These HT1 mutants abrogate the CO₂-induced MPK4/12-HT1 interaction and HT1 inhibition. MPK12 kinase activity is not required for CO₂ sensor function, and CO₂-induced HT1 inhibition and stomatal closing. The presented findings reveal that MPK4/12 and HT1 together constitute the long-sought stomatal CO₂ sensor in plants.

Plant stomata open and close in response to changing environmental conditions thereby regulating gas exchange between plants and the atmosphere. CO₂ influx into leaves from the atmosphere is essential for plant photosynthesis. Stomatal conductance is regulated by dynamic and rapid stomatal movements (1-5). Plants sense diurnal dark/light-induced changes in the CO₂ concentration (Cᵢ) in the intercellular air spaces of leaves, thus causing opening and closing of stomata (5). Furthermore, the continuing rise in the atmospheric concentration is narrowing stomatal pores globally (1, 6). Elevation in the leaf CO₂ concentration causes rapid stomatal closing, thus reducing transpirational water loss from plants. Conversely, in response to low CO₂, stomata open and increase stomatal conductance. The stomatal CO₂ response, therefore, is critical for plant growth and regulates the water use efficiency of plants. CO₂-induced stomatal movements in dicots and monocots require catalytic carbonic anhydrase activity (7, 8). These carbonic anhydrases accelerate the catalysis of CO₂ entering through the plasma membrane to lipid membrane impermeable bicarbonate ions (HCO₃⁻) and protons. Data indicate that the accumulated bicarbonate ions are an important intracellular messenger in guard cells that mediate stomatal closure (7, 9-11). However, the primary CO₂/bicarbonate sensor has remained elusive. This sensor is required for regulation of early protein phosphorylation events that drive CO₂-regulated stomatal movements (9, 12-14).

Using infra-red thermal imaging, a CO₂-insensitive Arabidopsis mutant was isolated, and the causative gene was identified as a Raf-like protein kinase named High Leaf Temperaturel (HT1), suggesting an important role for protein phosphorylation in CO₂-induced stomatal movements (12). Recessive ht1-2 mutant stomata show a constitutively high CO₂-like closed stomatal phenotype regardless of the CO₂ concentration, but respond to blue light and the plant hormone abscisic acid (12). Furthermore, other Raf-like protein kinases CBC1 and CBC2 are essential for the stomatal CO₂ response (13). Since cbc1 cbc2 double mutants show constitutively closed stomata similar to the ht1-2 mutant, the HT1 and CBC kinases are considered to be negative regulators of high CO₂-induced stomatal closure, but the underlying CO₂ regulation mechanisms remain unknown.

Conversely, double mutant alleles in the Arabidopsis MPK4 and MPK12 MAP kinases show constitutively open stomata and insensitivity to high CO₂ concentrations, but an intact abscisic acid response, suggesting that these MAP kinases are redundant positive regulators of early CO₂ signal transduction in guard cells (14). However, the CO₂ sensor remains unknown and the subsequent signaling network mechanisms remain unclear. Here we reveal that the CO₂ sensor consists of the protein complex of MPK4/12 with the HT1 protein kinase. Elevated CO₂ causes a direct interaction of MPK4 & MPK12 with HT1, thereby directly inhibiting HT1 activity and downstream CBC1 activity. Moreover, MAP kinase activity is not required for CO₂ sensor signaling and CO₂-regulated stomatal movements.

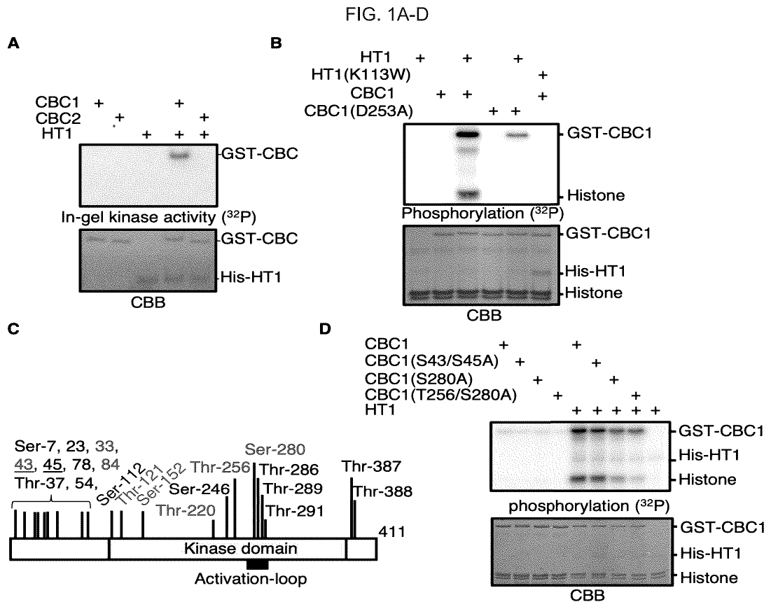

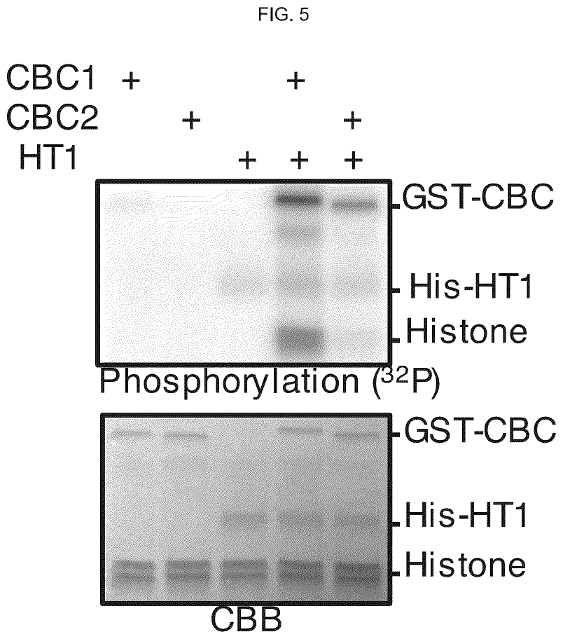

A previous study reported that the HT1 kinase phosphorylates the CBC1 kinase in vitro (13). Whether this phosphorylation of CBC1 affects CBC1 activity remains unknown. We confirmed CBC1 phosphorylation by HT1 using recombinant His-HT1 and GST-CBC1 proteins by in vitro phosphorylation assays using radioactive ³²P-ATP (FIG. 5). Moreover, phosphorylation levels of histone, an artificial kinase substrate, were increased at the same time. Together with findings that histone is not a substrate of HT1 (for example FIG. 5), these data suggest that the CBC1 phosphorylation by HT1 may induce CBC1 kinase activation (FIG. 5). In-gel kinase assays were pursued to test this hypothesis and provide direct evidence of HT1-dependent CBC1 kinase activation (FIG. 1A). In contrast, the kinase inactive HT1-K113W mutant did not activate CBC1 (FIG. 1B). The kinase inactive CBC1-D253A isoform shows a reduced phosphorylation level compared to wild type CBC1 and no clear phosphorylation of histone in the presence of HT1 (FIG. 1B). These findings suggest that after CBC1 activation by HT1, the CBC1 protein kinase also mediates auto-phosphorylation of CBC1 and trans phosphorylation of histone (FIG. 1B). We identified in vitro phosphorylation sites in CBC1 using mass spectrometry by analyzing recombinant CBC1 protein in the presence or absence of HT1 (FIG. 1C). We found two HT1-dependent phosphorylation sites (Thr-256 and Ser-280) that lie within or near the activation-loop of CBC1. In vitro phosphorylation assays suggest that these two HT1-dependent phosphorylation sites play an important role in HT1-mediated CBC1 activation (FIG. 1D). In contrast, the blue-light dependent phosphorylation sites (Ser-43 and 45) (13) do not have a clear role in HT1-mediated CBC1 activation (FIG. 1D).

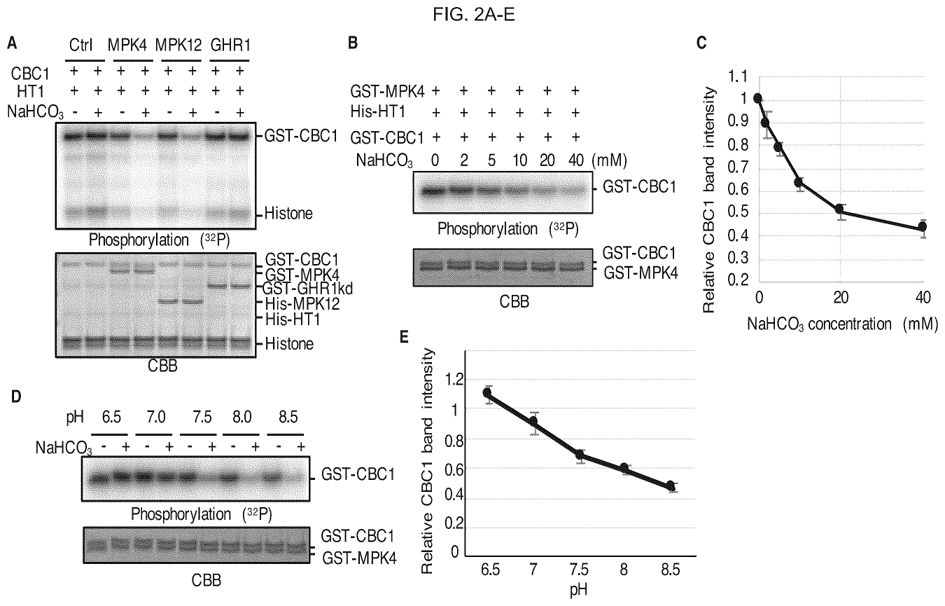

We tested whether this HT1-mediated activation of CBC1 is inhibited by CO₂/bicarbonate by adding NaHCO₃ in in vitro phosphorylation reactions. However, our results show no clear effect of NaHCO₃ on the CBC1 phosphorylation level (FIG. 2A, control lanes, n=3 experiments). Surprisingly, when MPK4 or MPK12 were added to the reaction, we found that the addition of NaHCO₃, but not NaCl, inhibited both CBC1 phosphorylation and histone phosphorylation in vitro (FIG. 2A, MPK4/12 lanes, n>6). However, we did not observe a clear effect of MPK4 or MPK12 without addition of NaHCO₃(FIG. 2A, MPK4/MPK12 lanes). In contrast, the cytosolic domain of the (pseudo-) receptor kinase GHR1 (15, 16) had no clear effect, further indicating a function of MPK4/12 (FIG. 2A, GHR1 lanes).

The inhibitory down-regulation of CBC1 activity shows a NaHCO₃ dose-dependency (FIGS. 2B and C). The ECso was ˜7.1+/−1.0 mM, which is similar to the unrelated cyanobacterium adenylyl cyclase bicarbonate sensor (17) and the mammalian soluble adenylyl cyclase bicarbonate sensor (18). In control “—NaHCO₃”-free experiments, we used the same concentration of NaCl, which did not cause CBC1 activity regulation (FIG. 2). When in vitro phosphorylation assays were performed using reaction buffers adjusted to different pH values individually, NaHCO₃ inhibited CBC1 phosphorylation in the presence of MPK4 and HT1 under high pH conditions at ≥pH 7.5 (FIGS. 2D and E), which suggests that bicarbonate ions are the main inorganic carbon signaling species. We note these results do not necessarily exclude a secondary role of CO₂, which is more abundant at low pH, although bicarbonate ions clearly have the stronger effect on kinase regulation (FIGS. 2D and E). A human soluble adenylyl cyclase, for example, senses both CO₂ and bicarbonate ions (19).

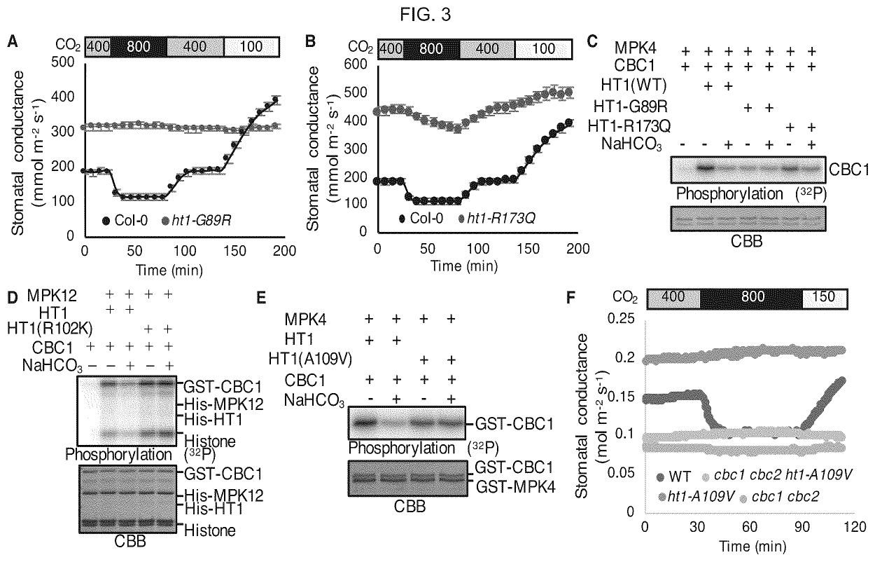

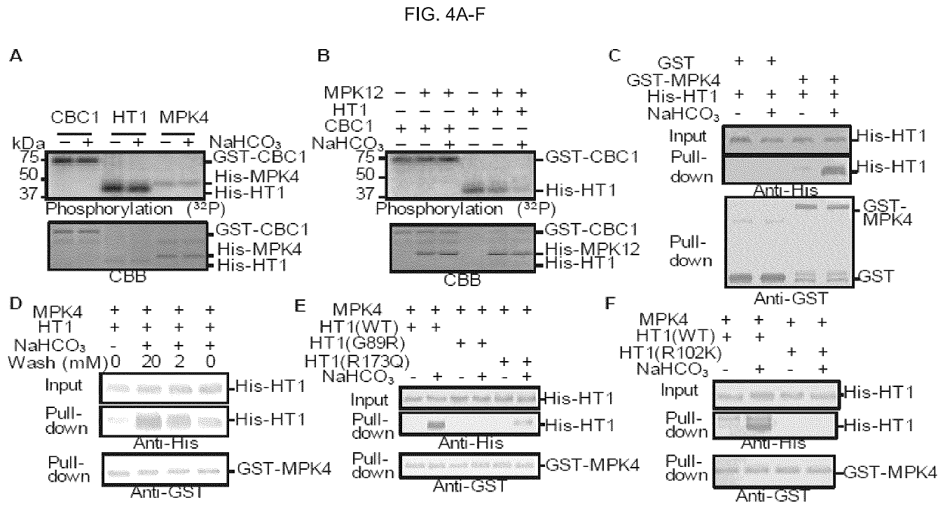

In parallel to these analyses, a genetic screen was pursued for ozone sensitive Arabidopsis mutants, which can result from higher stomatal conductance mutant phenotypes that enable damaging access of ozone into intercellular leaf spaces (20). Screening of greater than 50,000 EMS mutagenized M2 generation Arabidopsis lines led to isolation of candidate mutants impaired in the stomatal high CO₂ response, while exhibiting intact abscisic acid-induced stomatal closing (see Methods). These mutants included 6 mutants in the HT1 gene, comprising new ht1-G89R and htl-R173Q) alleles and re-isolations of the known htl-A109V variant in four remaining mutants. All of these ht1 mutant alleles were dominant and showed stomatal insensitivity to CO₂ elevation (FIG. 3A,B and F). Whole plant gas exchange analyses revealed that the ht1-G89R mutant showed an increased stomatal conductance at ambient CO₂ that did not respond to changes in the CO₂ concentration (FIG. 3A). The ht1-R173Q) mutant showed partly impaired stomatal conductance responses to CO₂ shifts (FIG. 3B).

Interestingly, in vitro phosphorylation assays revealed that the HT1-G89R isoform shows no NaHCO₃-mediated inhibition of CBC1 activity, in contrast to the WT HT1 protein (FIG. 3C, n=4 experiments). Furthermore, the R173Q mutation partly impaired the NaHCO₃-dependent CBC1 downregulation (FIG. 3C). These results are consistent with the stomatal phenotypes of the mutant plants (FIG. 3A to C). The smaller stomatal conductance of the ht1-G89R mutant plants than that of WT plants in response to low CO₂ conditions (FIG. 3A) is likely due to the lower kinase activity of the HT1-G89R isoform at low CO₂/bicarbonate concentrations (FIG. 3C). We examined additional dominant ht1 mutants. In contrast to recessive ht1 kinase mutants, two dominant HT1 mutations, HT1-R102K and HT1-A109V, cause a constitutively open high CO₂-insensitive stomata phenotype (21, 22), similar to the newly identified ht1-G89R allele (FIG. 3A). These data suggest that these dominant mutations constitutively enhance HT1 function in guard cells. However, these mutations do not greatly enhance HT1 kinase activity (21, 22). Interestingly, in our phosphorylation assays using MPK4/12, HT1 and CBC1 recombinant proteins, both of these R102K and A109V mutations disrupt the NaHCO₃-triggered down-regulation of CBC1 phosphorylation and CBC1 activity (FIG. 3D and E, n=3 (D) and n=4 (E) experiments).

The above results suggest that our in vitro signaling analyses can explain how these HT1 point mutations confer their CO₂-insensitive stomatal phenotypes. In a model derived from the above findings, low CO₂-induced activation of the CBC1 kinase requires CBC1 phosphorylation by HT1. CBC1 activity in turn is down-regulated by high CO₂/bicarbonate down-regulation of HT1. A prediction of this model would be that the constitutively open stomatal phenotypes of the dominant ht1-A109V mutant would require the presence of the CBC kinases. We created che1 che2 ht1-A109V triple mutant plants. Stomatal conductance analyses show that the triple mutant has a closed stomatal phenotype, similar to che1 cbc2 mutant leaves, whereas ht1-A109V single mutant leaves have constitutively open stomata (FIG. 3F) This genetic evidence supports the model that HT1 provides a critical upstream regulator connecting CO₂ sensing to downstream CBC kinase activity regulation.

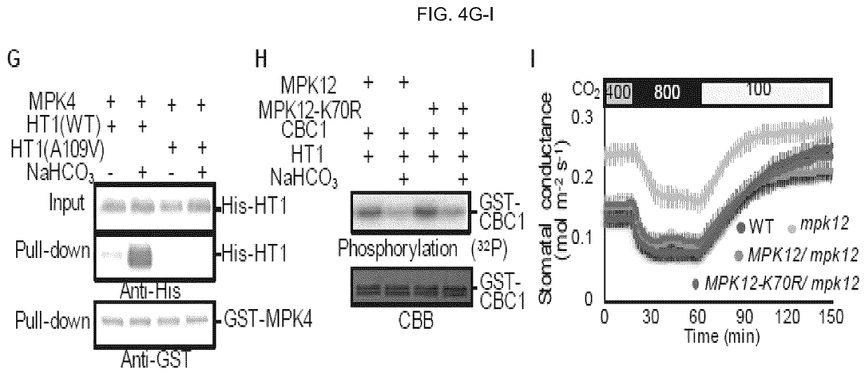

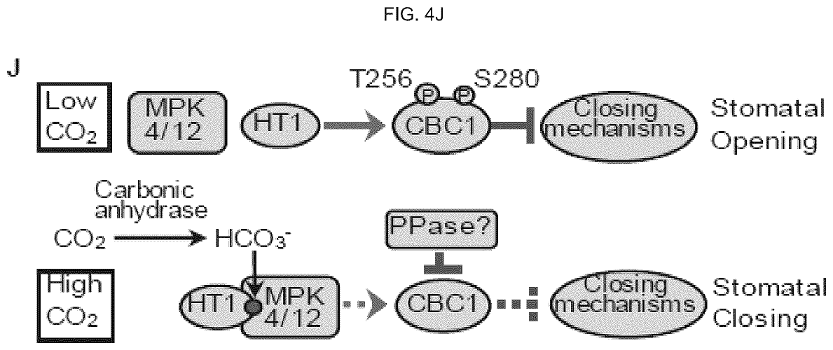

We further pursued experiments to identify the molecular mechanism of CO₂/bicarbonate sensing. When CBC1, HT1 or MPK4 were exposed to high NaHCO₃ individually, their kinase activities were not affected (FIG. 4A), in the case of the MPKs, consistent with previous findings showing no direct MPK4 and MPK12 activation by elevated CO₂ or NaHCO₃ (14). However interestingly, HT1 kinase activity was inhibited in response to NaHCO₃, only in the presence of MPK12 (FIG. 4B; n=3). In contrast, CBC1 activity was not affected when only MPK12 and CBC1 were added to the reaction (FIG. 4B; n=3), suggesting that MPK12 and HT1 might be the bicarbonate sensing module.

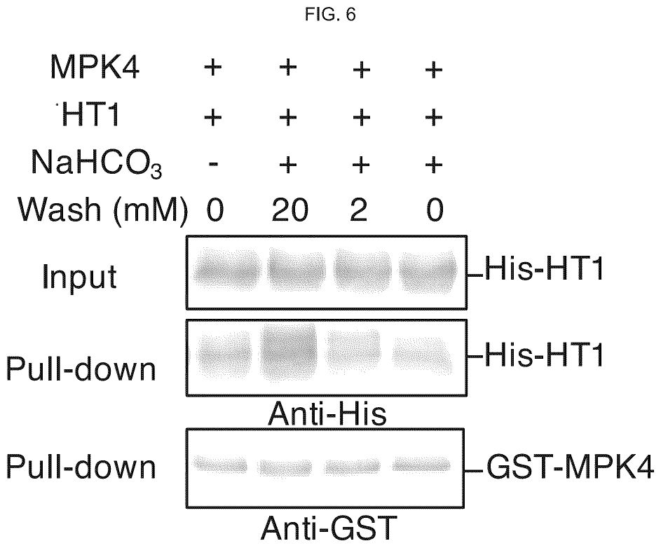

We therefore investigated a possible binding between MPK4 and HT1 and the effect of HCO₃⁻. In vitro pull-down assays showed that HCO₃⁻ greatly enhanced the binding between MPK4 and HT1 (FIG. 4C, n>6 experiments). This HCO₃⁻-dependent binding was reversed by removing HCO₃⁻, indicating that MPK4 and HT1 interact reversibly depending on the bicarbonate concentration (see FIG. 4D, FIG. 6).

Interestingly, the HT1-G89R mutation disrupts the HCO₃⁻-dependent binding with MPK4 (FIG. 4E, n=3 experiments). The HT1-R173Q isoform, that causes a weaker CO₂-insensitive phenotype (FIG. 3B), is still able to partially interact with MPK4 upon HCO₃⁻ addition, albeit less strongly than the wildtype MPK4-HT1 proteins (FIG. 4E, n=3 experiments). Furthermore, the strong dominant HT1-R102K and HT1-A109V mutant isoforms did not show a bicarbonate-induced interaction of HT1 with MPK4 (FIG. 4F and G, n=3 experiments). These findings are consistent with in vitro phosphorylation assays (FIG. 3C to E) and stomatal conductance analyses (FIG. 3A, B and F). These results suggest that MPK4/12 and HT1 are the long-sought CO₂/bicarbonate sensor, for which HCO₃⁻ causes an interaction of MPK4/12 with HT1, which in turn inhibits the negative regulatory HT1 protein kinase activity, thus enabling high CO₂-induced stomatal closure to proceed.

Surprisingly, we found that the kinase inactive MPK12-K70R isoform (23) retained the ability to mediate CO₂/bicarbonate-induced CBC1 inhibition via the HT1 protein kinase in in vitro phosphorylation assays (FIG. 4H; n=4). We further investigated the requirement of MPK activity for CO₂ regulation of stomatal movements in planta. Strong mpk12 mutant alleles show a larger steady state stomatal conductance and slightly slowed CO₂ responses (14, 22, 23). Consistent with phosphorylation analyses (FIG. 4H), the inactive MPK12-K70R isoform rescued the open and slowed stomatal CO₂ response phenotype of the mpk 12 mutant (FIG. 4I). Complementation of the in planta mpk12 CO₂ response was indistinguishable upon expression of the kinase dead MPK12-K70R kinase or the wildtype MPK12 isoforms. These findings further suggest that the signaling mechanism by which the HT1 and MPK12 protein kinases sense CO₂ concentration functions via a reversible MPK-HT1 interaction rather than HT1 phosphorylation by these MAP kinases.

In this study, we reveal the biochemical, genetic and physiological stomatal CO₂ sensing and early signaling core mechanisms that use three types of protein kinases, MPK4/12, HT1 and CBC1. While HCO₃⁻ can modulate ˜20% of the activity of downstream S-type anion channels, as a proposed secondary HCO₃⁻ response mechanism (10), the primary CO₂/bicarbonate sensor that controls the required upstream phosphorylation events and thereby stomatal closing, has remained elusive. The present findings that the MPK4/MPK12-HT1 complex functions as a bicarbonate sensor together with strong genetic CO₂-insensitive phenotypes of the respective ht1 and cbc1 2 mutants (FIGS. 3A, B and F) (12, 13) explains how plant cells sense and transmit the CO₂ signal to trigger stomatal closure. At low CO₂/bicarbonate concentrations, the HT1 kinase phosphorylates and activates the CBC1 protein kinase, which leads to inhibition of stomatal closing mechanisms (FIG. 4J). However, when guard cells are exposed to high CO₂ concentrations, carbonic anhydrases accelerate the intracellular conversion of CO₂ to bicarbonate (7, 8), and the accumulated bicarbonate ions can trigger MPK4/12-HT1 binding that leads to inhibition of HT1 kinase activity. HT1 kinase inhibition in turn results in down-regulation of CBC1 kinase activity promoting induction of stomatal closure (FIG. 4J). The kinase inactive MPK12 isoform is sufficient for stomatal CO₂ sensing and the in planta complementation of the stomatal CO₂ response (FIGS. 4H and I), suggesting an unexpected phosphorylation-independent MAP kinase function in plants. The reversible MPK4/12-HT1 binding (FIG. 4D, FIG. 6) further correlates with the rapid reversibility of stomatal opening and closing in response to changing CO₂ concentrations in leaves (1, 3). The identification of the guard cell CO₂ sensing and the mechanisms within this CO₂ signaling core that regulates stomatal conductance can lead to future targeted engineering of plant water use efficiency and carbon intake in light of the continuing increase in the atmospheric CO₂ concentration (2, 6, 24, 25).

### FIGURE LEGENDS

FIG. 1. The CO₂ signaling Raf-like kinase CBC1 is activated by the HT1 protein kinase through phosphorylation.
(A) Recombinant CBC1 and CBC2 proteins were incubated with or without HT1 proteins for 30 min with cold ATP, and in-gel kinase assays were performed.
(B) The kinase inactive CBC1-D253A and HT1-K113W protein isoforms were used for in vitro phosphorylation analyses with recombinant CBC1 and HT1 proteins as indicated (see text). Histone was used as an artificial phosphorylation substrate of CBC1.
(C) Recombinant CBC1 proteins were incubated with or without HT1 and ATP, and CBC1 phosphorylation sites were identified by mass spectrometry. The red fonts indicate HT1-dependent phosphorylation sites. CBC1 autophosphorylation sites detected without HT1 addition are labeled in black fonts. The underlined Ser-43 and Ser-45 were previously reported as blue light-dependent phosphorylation sites (13), but did not affect HT1 activation of CBC1 (D).
(D) CBC1-S43/S45A, S280A and T256/S280A proteins were used for in vitro phosphorylation assays. CBB gels show loading controls.

FIG. 2. MAP kinases MPK4 and MPK12 inhibit HT1-mediated CBC1 kinase phosphorylation in the presence of NaHCO₃ in vitro.
(A) Recombinant HT1 and CBC1 proteins were incubated with MPK4, MPK12 or the (pseudo)-kinase domain of GHR1 in the presence or absence of 20 mM NaHCO₃ for 30 min, and in vitro phosphorylation assays were performed. Histone was used as an artificial protein kinase substrate.
(B) MPK4, HT1 and CBC1 proteins were incubated with NaHCO₃ at the indicated concentrations for 30 min, and in vitro phosphorylation assays were performed.
(C) CBC1 band intensities as shown in (B) were measured using IMAGEJ™, n=4 experiments, error bars show ±S.D.
(D) MPK4, HT1 and CBC1 proteins were incubated in reaction buffers adjusted at different pH (6.5 to 8.5) for 30 min, and in vitro phosphorylation assays were performed.
(E) CBC1 band intensities and the density-ratios of ‘+NaHCO₃’ to ‘—NaHCO₃’ (=+NaCl controls) for each pH condition

FIG. 3. The dominant HT1 mutations (HT1-R102K and A109V) disrupt HCO₃⁻-dependent downregulation of CBC1 protein kinase activity.
(A) and (B), Whole plant gas exchange analyses using ht1-G89R (A) and ht1-R173Q
(B). Ambient CO₂ concentrations are indicated by the top bars. n=7 experiments, error bars show ±SEM.
(C) Recombinant HT1 (wild type, HT1-G89R or HT1-R173Q) and CBC1 proteins were incubated with MPK4 in the presence or absence of 20 mM NaHCO₃ or 20 mM NaCl (−controls) for 30 min, and in vitro phosphorylation assays were performed.
(D) Recombinant MPK12, CBC1 and HT1 (WT or R102K) proteins were incubated with or without 20 mM NaHCO₃ or 20 mM NaCl (−controls) for 30 min, and in vitro phosphorylation assays were performed. Histone was used as an artificial kinase substrate.
(E) HT1 (WT or A109V) proteins were used for in vitro phosphorylation assays. Proteins were incubated with or without 20 mM NaHCO₃ or 20 mM NaCl (−controls) for 30 min.
(F) Stomatal conductances were analyzed using intact plants of Arabidopsis (Col-0 (WT), cbc1 cbc2, ht1-A109V and cbc1 cbc2 ht1-A109V). CO₂ concentration changes were applied as indicated on top (p.p.m.).

FIG. 4. Bicarbonate inactivates HT1 kinase by stabilizing HT1 interaction with MPK4/12.
(A) Recombinant CBC1, HT1 and MPK4 proteins are incubated with or without 20 mM NaHCO₃ for 30 min, and in vitro phosphorylation assays were performed.
(B) CBC1 and HT1 proteins were incubated with or without 20 mM NaHCO₃ or 20 mM NaCl (“−” controls) in the presence or absence of MPK12 protein.
(C) His-HT1 and GST-MPK4 or GST control proteins were used for in vitro pull-down assays with or without 20 mM NaHCO₃. NaHCO₃ or 20 mM NaCl (“−” controls) were supplemented in all buffers throughout the pull-down assay procedures including the washing step.
(D) In vitro pull-down assays showed reversibility and were performed using the washing buffers supplemented with NaHCO₃ at the indicated concentrations (0, 2 or 20 mM).
(E) In vitro pull-down assays were performed using recombinant HT1 (wild type, HT1-G89R and HT1-R173Q) proteins.
(F) In vitro pull-down assays were performed using HT1-R102K isoform.
(G) In vitro pull-down assays were performed using HT1-A109V isoform.
(H) In vitro phosphorylation assays were performed after CBC1, HT1 and MPK12 or the MPK12-K70R kinase inactive isoform proteins were incubated with or without NaHCO₃ or NaCl (“−”) controls.
(I) Stomatal conductances were analyzed in leaves of intact Arabidopsis plants [Col-0 (WT), mpk 12, pGC1:MPK12-GFP/mpk12 and pGC1:MPK12 (K70R)-GFP/mpk12]. CO₂ concentration changes were applied as indicated on top (p.p.m.). n=6 experiments, error bars show ±SEM.
(J) Model of plant stomatal CO₂ sensor and signaling (see text).

FIG. 5. The Raf-like kinase CBC proteins are phosphorylated by HT1 protein kinase.

In vitro phosphorylation analyses were performed using recombinant CBC1 and CBC2 proteins in the presence or absence of HT1. Histone was used as an artificial phosphorylation substrate of CBC1 (see main manuscript).

FIG. 6. Repeat experiment example of reversible interaction between MPK4 and HT1 in vitro.: In vitro pull-down assays using recombinant GST-MPK4 and His-HT1 proteins were performed with or without 20 mM NaHCO₃. Precipitated proteins were washed with buffers supplemented with NaHCO₃ at the indicated concentrations (0, 2 or 20 mM). See FIG. 4d for an independent experiment. To control for effects of Na⁺, 20 mM NaCl (0 mM NaHCO₃) for lanes 1 and 4 were included in the buffers; and 18 mM NaCl (2 mM NaHCO₃) for lane 3 were included in buffer.

### Example 2: Genetically Engineered Monocot Plants

This example describes studies using the grass monocot Brachypodium distachyon for CO₂ impaired mutants in controlling water loss. This example demonstrates that methods and genetically engineered monocot plants as provided herein are effective and can be used to engineer water transpiration and water use efficiency in plants, and engineered plants, plant tissues and cells as provided herein have increased water use efficiency and are drought-resistant.

We identify a major gene in this screen, map the gene and discover that it is the closest ortholog of the Arabidopsis CO₂ sensor component, MPK12 and MPK4. We also include data that the Brachypodium distachyon protein can function in the CO₂ sensor.

In summary these findings provide evidence that methods as provided herein also applies to grasses (monocots), which represents major staple crops such as maize, wheat, rice, barley etc.

Plants respond to increased CO₂ concentrations through rapid stomatal closure which can contribute to increased water use efficiency (WUE). Grasses have been shown to display faster stomatal responses than dicots due to the dumbbell shaped guard cells flanked by subsidiary cells working in opposition to facilitate stomatal movements. However, forward genetic screening for mutants in stomatal CO₂ signal transduction in grasses have not been reported. The grass model Brachypodium distachyon shows a high evolutionary similarity and collinearity to staple cereal crops.

To gain insights into genes and mechanisms that function in CO₂ control of stomatal movements in grasses, a forward genetics screen was developed and conducted on a population of individually stored M5 generation EMS (ethyl methyl sulfonate) mutagenized Brachypodium distachyon plants using infrared thermal imaging to identify plants with altered canopy leaf temperature at elevated CO₂. One of the mutant lines selected in this screen, chill1, was shown to be colder than wildtype parent Bd21-3 controls after exposure to increased CO₂ concentrations. Moreover, chill1 plants were strongly impaired in high CO₂-induced stomatal closing, while retaining a robust stomatal closing response to abscisic acid (ABA). Through the use of genetic and bulked segregant whole genome sequencing analyses as well as the generation and characterization of CRISPR-cas9 plants, chill1 was mapped to a MAP (mitogen activated protein) kinase gene. Biochemical reconstitution analyses and AlphaFold2-directed structural modeling demonstrate the MAP kinase is a key signaling component involved in CO₂-induced stomatal movements and functions as a component of the CO₂ sensor in grasses.

Brachypodium distachyon is emerging as a new model organism for monocots as opposed to the widely investigated model organism Arabidopsis thaliana, which has been shown to have very limited genomic collinearity to staple cereal crops (Brkljacic et al., 2011; Keller & Feuillet, 2000 Scholthof et al., 2018; Vogel and Bragg., 2009). In contrast to Arabidopsis, B. distachyon is much more closely related to cereal crops with a high degree of evolutionary collinearity (Hasterok et al., 2022; Sun et al., 2017). B. distachyon compared to other monocotyledonous models has a smaller size, compact genome, and short lifespan; making it favorable for high throughput screening (Brkljacic et al. 2011; Huo et al., 2009; Scholthof et al. 2018).

In the present study, we screened over 1000 M5 generation EMS-mutagenized B. distachyon lines for impairments in the stomatal CO₂ response. We report isolation of a mutant, chill1, that strongly impairs high CO₂-induced stomatal closure and show that this mutant on average has a higher stomatal conductance and disrupted response to increased CO₂ while retaining robust abscisic acid (ABA)-induced stomatal closing. Data suggest that chill1 encodes a central component of CO₂ regulation of stomatal movements in grasses.

### Results

Rapid stomatal closure is induced by exposure to elevated CO₂, limiting gas exchange and evapotranspiration, causing higher leaf temperatures which can be measured through infrared thermal imaging (Mustilli et al., 2002; Hauser et al., 2019). Infrared imaging was applied to a population of individually stored EMS mutagenized M5 generation B. distachyon to screen for plants with reduced sensitivity to altered CO₂ by selecting plants appearing cooler than the wildtype control (Bd 21-3) via infrared thermal imaging.

Through an infrared leaf thermal imaging screen conducted at ambient CO₂ (˜415 ppm) of 1,075 individually stored M5 generation EMS mutagenized B. distachyon lines, we isolated 50 putative “chill” mutants with leaf temperatures cooler than the WT parent (Bd21-3). After re-screening five plants from each of the individually stored M5 seed stock in a secondary screen, 28 of the 50 putative mutants showed cool leaves at elevated CO₂ (900 ppm). In a tertiary screen in the following (M6) generation, 7 of the 28 mutants could not be confirmed as markedly different from WT controls through thermal imaging analyses and were removed as candidates. The remaining 21 mutants were then named the chill mutant lines and reconfirmation for these 21 mutants was pursued by rescreening in the M6 generation.

Of the mutants identified in this screen, we initially focused on a mutant that we have named chill1 (FIG. 1A), which consistently and reliably appears cooler than WT (Bd 21-3) controls in infrared thermal imaging following exposure to increased CO₂ (1000 ppm) (FIG. 1A). To determine whether the difference observed in canopy leaf temperature was related to stomatal development or stomatal movements, stomatal index and density analyses were conducted comparing chill1 to the parental WT control (Bd21-3). No significant difference could be observed in stomatal index and density (FIGS. 1 B and 1C).

Stomatal conductance analyses were pursued (FIG. 1D) wherein chill1 shows a dramatically reduced response to elevation in CO₂ when compared to WT controls. We next investigated stomatal closure in response to abscisic acid. Interestingly, chill1 leaves responded similarly to WT following the addition of 2 μM abscisic acid (ABA) to the transpiration stream of 5-week old leaves (FIG. 1E).

To determine inheritance patterns, chill1 plants were backcrossed to the parental strain Bd 21-3 (see Methods). The F1 chill1×Bd21-3 population was initially analyzed by infrared thermal imaging experiments following exposure to increased (1000 ppm) CO₂. F1 crossed chill1×Bd21-3 plants exhibited a phenotype more similar to that of WT (Bd21-3 controls) (FIG. 2A). These data were consistent across four individual chill1×Bd 21-3 F1 plants. F1 plants were also analyzed in stomatal conductance analyses to quantify real time responses to shifts in CO₂ concentrations. The F1 backcrossed plants exhibited a WT-like response to high CO₂ (900 ppm) consistently across three biological replicates (FIG. 2B).

Tillers were created from these F1 plants (O'Connor et al. 2017) to generate large numbers of seeds for an F2 mapping population (Figure S1). The 4 week old F2 mapping population of approximately 560 individual F2 plants was screened following 1000 ppm CO₂ exposure. Screening was done with a maximum of three F2 plants alongside the WT as a control for all plants that were screened (FIG. 3). In an initial analysis of all infrared thermal images of F2 plants, 116 of approximately 560 plants screened showed a chill1-like phenotype (21%), which is close to a 25% to 75% segregation. Analyses of F1 and F2 generations suggest that the chill1 phenotype is due to a single locus recessive mutation. All images were also independently reanalyzed by three laboratory members in a very conservative manner to reduce false positive selection in bioinformatic mapping analyses. Of the plants screened, 57 plants (10%) were individually selected by all members as clearly showing a chill1-like phenotype and 50 plants (9%) were selected to be strongly wildtype-like to be used as a point of comparison in bioinformatic analyses. The 57 plants selected as chill1-like were named M-pool (mutant pool) and were screened a second time for those which exhibited the strongest phenotype. A smaller mutant pool of 25 individuals with the strongest chill leaf canopy phenotype was named SM-pool (Strongest Mutant Pool).

DNA from those selected to be chill1-like or WT-like was pooled and submitted for whole genome sequencing (WGS) to be utilized in Bulked Segregant Analyses (BSA) (Michelmore and Kesseli; 1991). Quality checks were performed on sequence reads before use in further analyses (Figure S2). QTLSEQR™ (Mansfeld and Grumet, 2018) was used to analyze files and generate visual representations of the −log₁₀ (p-value) derived from G′ values (Magwene et al 2011) (FIG. 4) (see Methods), which are generated based on SNPs called during analysis (Takagi et al. 2013). BSA analysis of both the mutant (M)-pool and the SM-pool show likely heterozygous and homozygous variants on chromosome 3 (Bd3) with some smaller potential peaks on other chromosomes (FIG. 4). Most of the called variants were heterozygous. We filtered the variant data for homozygous variants and compared the called variants in the M-pool and SM-pool vs. the wildtype-like pool. Called homozygous variants from short read WGS in both the M-pool and SM-pool were analyzed for variants predicted to cause impactful mutations were compiled into a single list via SNPEFF™ (an open source tool that annotates variants and predicts their effects on genes by using an interval forest approach, see Cingolani P, et al. Fly (Austin). 2012 April-June; 6 (2): 80-92) and QTLSEQR™ (an R package for Quantitative Trait Locus (QTL) mapping using next-generation sequencing (NGS) Bulk Segregant Analysis, see for example, Cingolani et al., 2012; Mansfeld and Grumet, 2018). Heterozygous variants were excluded from further analyses on the basis that chill1 behaves like a recessive mutant as determined by the FI generation phenotype and F2 generation segregation. After variants were filtered for homozygosity four variants were called for the mutant (M) pool and three for the strongest mutant (SM) pool. Variants in two genes (BdiBd21-3.3G0222500 and 3G0296900) were called in both pools leading to a total compiled list of five possible candidate genes (FIG. 5A).

To reduce the number of potential candidates, additional chill1 F2 plants were screened and those selected as cooler than WT controls were backcrossed to WT Bd 21-3 a second time. Plants were taken into the F4 generation and were rescreened by infrared thermal imaging. DNA was extracted from those selected individually by two different members to be cooler than WT (Bd 21-3) controls. DNA from selected F4 plants was used to confirm the presence of called variants via Sanger sequencing. Of the remaining five candidate genes, four were excluded from further analyses based on the absence of the called variant or heterozygosity at the called loci, confirmed via Sanger sequencing. The only candidate which could not be excluded was in the BdiBd21-3.3G0222500 gene. The called variant in exon 2 was confirmed by Sanger sequencing and found to be homozygous in 11 individually analyzed chill1-like F4 plants but was not found in three WT (Bd21-3) controls nor in a F4 plant selected as WT-like (FIG. 5B). These analyses suggested that the variant in BdiBd21-3.3G0222500 may be responsible for the chill1 phenotype.

To further test this hypothesis, we searched the Phytozome (phytozome-next.jgi.doe.gov) database for sequence-indexed sodium azide (NaN) mutagenized lines (Dalmais et al, 2013) predicted to contain a mutation in BdiBd21-3.3G0222500. The NaNO770 line was obtained and was sequenced to confirm the presence of the predicted T to C point mutation sustained at position 14916843 on chromosome Bd3 via Sanger sequencing. NaN0770 plants in which the predicted mutation was homozygous and confirmed were analyzed by infrared thermal imaging alongside the original chill1 mutant allele, WT controls and F1 crossed plants of the NaN0770 line and WT Bd 21-3 plants (FIGS. 6A and 6B). Infrared imaging of the NaN0770 line showed canopy temperatures that were more similar to chill1 than to WT control plants (FIG. 6A). However, the NaN0770×wildtype F1 backcrossed line appeared more similar to WT controls (FIG. 6A). NaN0770 was also used in an allelism cross with chill1 wherein the F1 cross appeared cooler than WT (FIGS. 6C and 6D). Canopy temperatures in the NaNO770 line and in the F1 cross to chill1 were cooler than WT (Bd 21-3) controls but less pronounced than chill1. These data indicated that the NaN0770 line has a less severe phenotype compared to chill1 (FIGS. 6C and D).

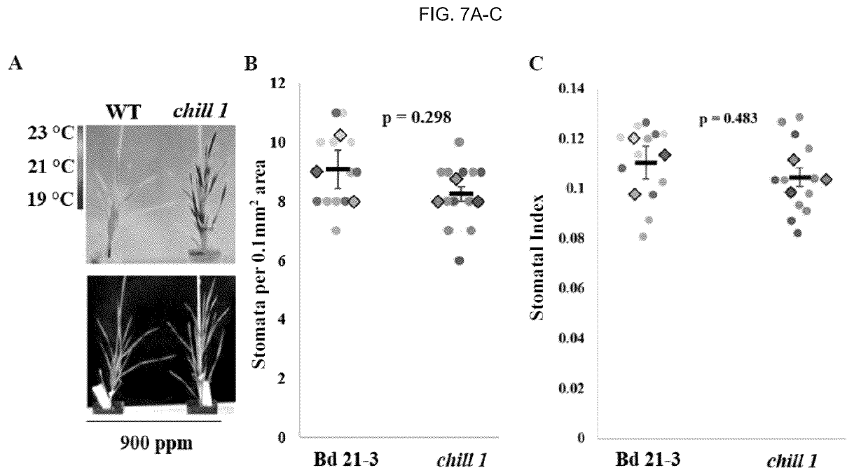

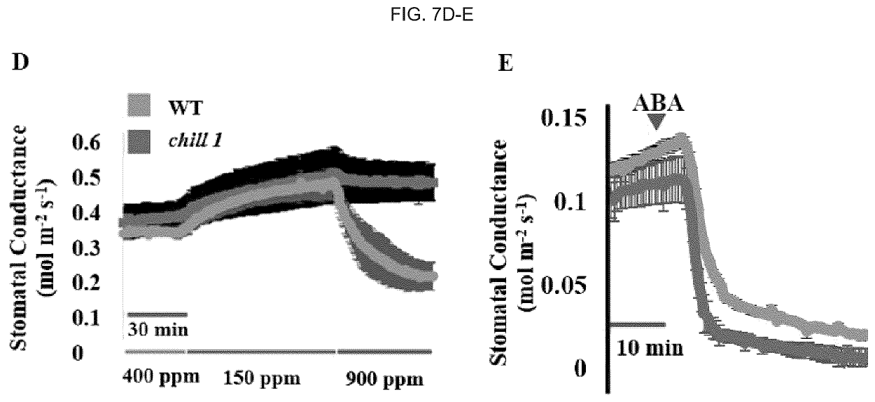

Taken together, the above data provide evidence that the chill1 variant in BdiBd21-3.3G0222500 may be responsible for the cool leaf canopy temperature. To further test this hypothesis, CRISPR-cas9 lines were generated using a guide RNA targeting BdiBd21-3.3G0222500, which lies in a gene annotated as BdMPK5 (FIG. 7A). BdMPK5 was amplified in both WT and CRISPR-cas9 lines and aligned for comparison to determine the presence of mutations sustained within the gene of interest leading to identification of individual CRISPR alleles in in BdiBd21-3.3G0222500. Individual T2 generation CRISPR plants were also genotyped to detect the presence or absence of Cas9. T2 CRISPR plants were additionally genotyped to confirm mutations sustained within BdMPK5. Only plants which were confirmed to be Cas9-free (Figure S3) and to have sustained predicted impactful mutations in BdMPK5 were taken into the following generation. Two independent CRISPR alleles were isolated and confirmed via Sanger Sequencing (FIG. 7B; CRISPR #1 and CRISPR #2).

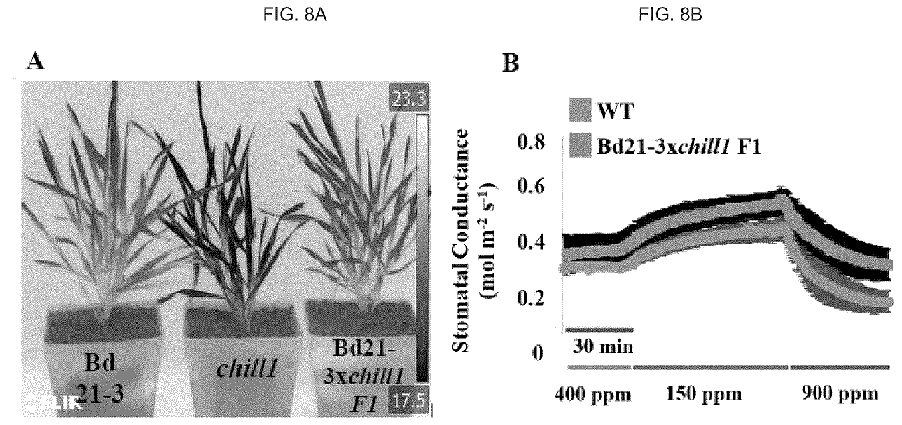

Stomatal conductance analyses were pursued in T3 CRISPR plants wherein both alleles exhibited a constitutively high stomatal conductance which did not decrease in response to shifts to high CO₂ conditions (800 ppm) (FIG. 8A). In further stomatal conductance analyses, 2 mM ABA was added to the transpiration stream and a steep decline in conductance was observed in T3 CRISPR lines (FIG. 8B). Genotype blinded stomatal index and density analyses were pursued for these lines and while the average stomatal density of CRISPR #1 line appeared lower when compared to WT parent controls, a significant difference could not be discerned with a significance cutoff of p≤0.05 for a 95% confidence interval (FIGS. 8C and 8D).

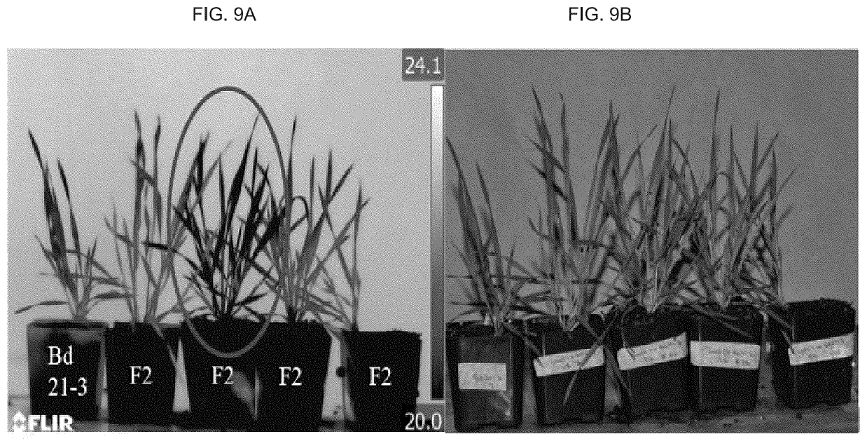

Blast analysis against Arabidopsis thaliana genes suggested that the mapped BdiBd21-3.3G0222500 (BdMPK5) gene encodes a close B. distachyon ortholog of the A. thaliana AtMPK4 and AtMPK12 proteins (AT4G01370 and AT2G46070 respectively). A recent study showed that the Arabidopsis AtMPK12 and AtMPK4 proteins function as part of the primary CO₂/bicarbonate sensor together with the HT1 protein kinase, that mediates CO₂ control of stomatal movements (Takahashi et al., 2022). Recent findings showed that HT1 activates the downstream protein kinase CBC1 by phosphorylating CBC1 at defined residues (Takahashi et al., 2022). Furthermore, this activation of CBC1 is inhibited by elevated CO₂/bicarbonate only when HT1, CBC1 and MPK4 or MPK12 are included in the reconstituted reaction (Takahashi et al., 2022). In-vitro protein kinase assays were pursued using the recombinant BdMPK5 protein to investigate whether BdMPK5 shows a CO₂ response together with HT1 and CBC1. HT1 and CBC1 together with the BdMPK5 protein showed a high CO₂/bicarbonate-induced down-regulation of the CBC1 protein kinase activity (FIG. 9, Figure S4; n=2). The high CO₂/bicarbonate response was similar to high CO₂ responses found for AtMPK4 and AtMPK12, but not for the close homolog AtMPK11 (Takahashi et al., 2022).

We next investigated whether the BdMPK5-D90N variant identified in chill1 has an effect on CO₂-mediated downregulation of CBC1 phosphorylation. The BdMPK5-D90N variant protein abrogated the high CO₂/bicarbonate-mediated downregulation of CBC1 phosphorylation (FIG. 9; n=2). These analyses showed that BdMPK5 can replace AtMPK4 and AtMPK12 in the reconstitution of this CO₂ response and that the chill1 variant in BdMPK5 cannot mediate downstream regulation of CBC1 protein phosphorylation (FIG. 9).

To gain insight into the mechanism by which the BdMPK5-D90N protein disrupts CO₂-induced stomatal closing in vivo (FIGS. 1,3, 8) and reconstitution of the CO₂ sensing core in vitro (FIG. 9A), we used AlphaFold2 (Jumper et al., 2021; Evans et al., 2022) to predict the BdMPK5-HT1 protein complex structure (FIG. 9B), using methods previously developed from the Arabidopsis MPK12/MPK4-HT1 sensor complex (Takahashi et al., 2022). Apart from the flexible loop regions, the predicted structure of the mutant BdMPK5-HT1 complex, with the BdMPK5-D90N mutation, is similar to to HT1 in the MPK12/MPK4-HT1 complexes predicted previously (Takahashi et al, 2022), displaying the reproducibility of these methods. Interesting, the mutant residue BdMPK5-D90N is predicted to lie directly at the interaction surface of BdMPK5 and HT1 (FIG. 9B).

FIG. 7 (or FIG. 1 of Example 2): chill1 mutant shows impaired stomatal response to changes in CO₂ but remains responsive to exogenous ABA:
(A) 4 to 5 week old Brachypodium distachyon plants were imaged following exposure to high CO₂ Pseudo-colored temperature scale is on the left. (top infrared thermal image, bottom photo of the same plants).
(B) Stomatal density and (C) stomatal index were determined using leaf imprints created of the 4^{th} true leaf of plants with 3 independent replicates plants per genotype with 4 images per plant. Circles represent counts per each image while diamonds are the average per each plant. Error bars represent SEM while p-values were obtained from two-tailed t-tests comparing mutant to WT.
(D) Stomatal conductance was quantified using a gas exchange analyzer. Data shown are the average of n=3 plants per genotype±SEM using 4 leaves per plant.
(E) Stomatal conductance was recorded prior to and following addition of 2 μM ABA. Data shown are the average of n=3±SEM plants per genotype using 1 leaf per experiment and genotype.

FIG. 8 (or FIG. 2 of Example 2): Characterization of chill1 F1 backcross:
(A) 5 to 6 week old backcrossed chill Brachypodium distachyon plants were analyzed (right) by infrared thermal imaging following 2 hour exposure to 1000 ppm CO₂ alongside parent line Bd 21-3 (WT) and the chill1 mutant. Pseudo-colored temperature scale is on the right.
(B) Stomatal conductance response of the F1 cross of the parent line Bd21-3 with the chill1 mutant was analyzed using a gas exchange analyzer. Data shown are the average of n=3±SEM experiments using 4 leaves per genotype in each experiment.

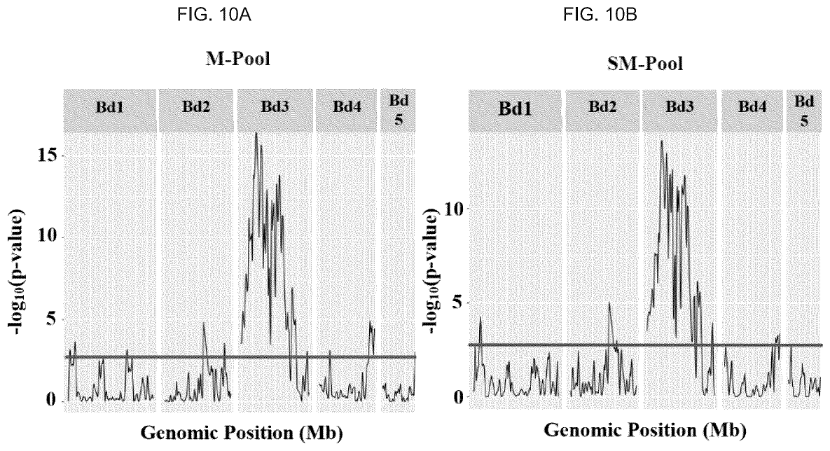

FIG. 9 (or FIG. 3 of Example 2):
(A) 4 to 5 week old chill1×Bd 21-3 F2 backcrossed plants were exposed to 1000 ppm CO₂ for 2 hours then immediately imaged using the infrared thermal imaging camera. The left most plant is the Bd21-3 parent line. The blue ellipse highlights a plant showing a chill1-like lower temperature. Pseudo-colored temperature scale is on the right.
(B) Color images were taken at the same time for plant identification. FIG. 10 (or FIG. 4 of Example 2): QTLSEQR™ output for bulk-segregant analysis for M- and SM-Pool, respectively

Bulk-segregant analysis was performed with two different data sets. The (left) M (mutant)-pool (57 chill1-like individuals) was used as the mutant pool and (right) the SM (strong phenotype mutant)-pool (37 chill1-like individuals with the most robust infrared thermal image phenotypes) were used as the mutant pools. In both analyses the WT-like pool (50 WT-like F2 individuals) was used as the reference. X-axis shows the chromosome number and genomic position. The Y-axis represents the −log 10 (p-value) derived from the G′ value (see Methods). The genome-wide false discovery rate of 0.01 is indicated by the red line.

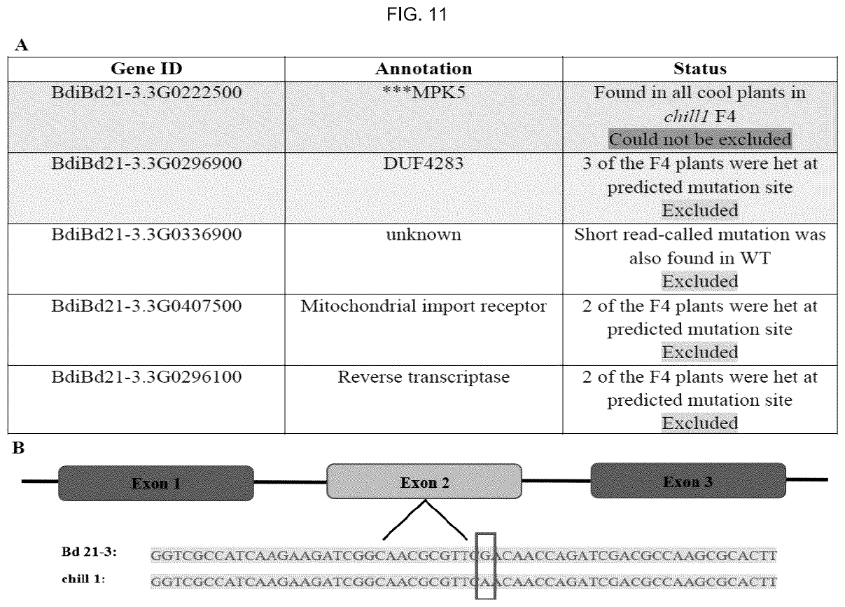

FIG. 11 (or FIG. 5 of Example 2): Candidate Genes Derived from BSA
(A) Variants in genes called as homozygous via Bulked Segregant analyses for the SM and M pools were compiled to create a list of called homozygous candidate mutations (see Results). Results of targeted Sanger sequencing are shown in the right column (het: heterozygous).
(B) The called homozygous mutation within the 2^{nd} exon of BdMPK5 (BdiBd21-3.3G0222500) is shown by the circled nucleotides and has been confirmed via sequencing.

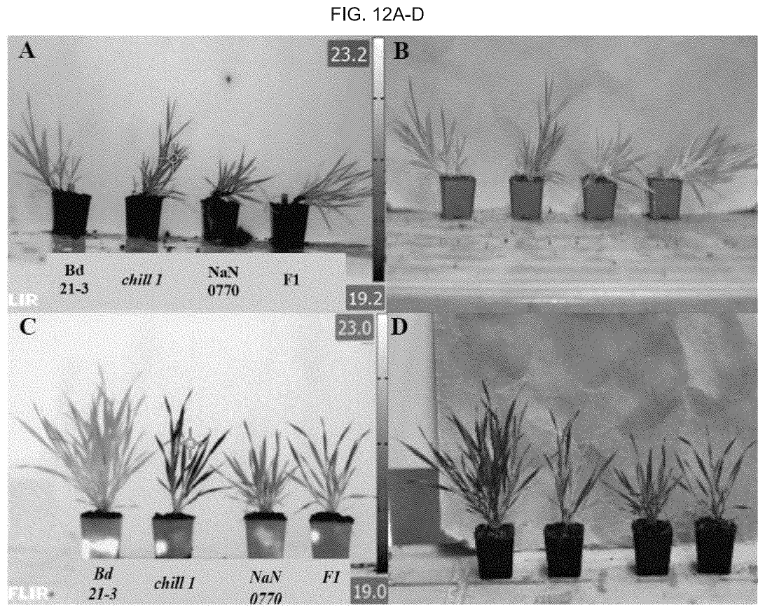

FIG. 12 (or FIG. 6 of Example 2): Sodium Azide Line 0770 crosses
(A) 4 to 5 week old chill1, sequence indexed and confirmed sodium azide (NaN) line and the Bd21-3× chill1 F1 backcross were exposed to 1000 ppm CO₂ for 2 hours alongside the Bd21-3 parent (WT) control.
(B) Color images were taken at the same time for identification of plants. Plants from left to right are labeled Bd21-3 (WT), chill1, NaN0770, and the F1 NaN0770 backcross.
(C) The sodium azide mutagenized NaN0770 line was crossed to chill1 and the F1 plants were used for infrared thermal imaging following exposure to high (1000 ppm) CO₂.
(D) Color image of the same plants. Plants from left to right are Bd 21-3, chill1, NaN0770 and the chill1×NaN0770 F1 cross. Pseudo-colored temperature scale is on the right in (A) and (C).

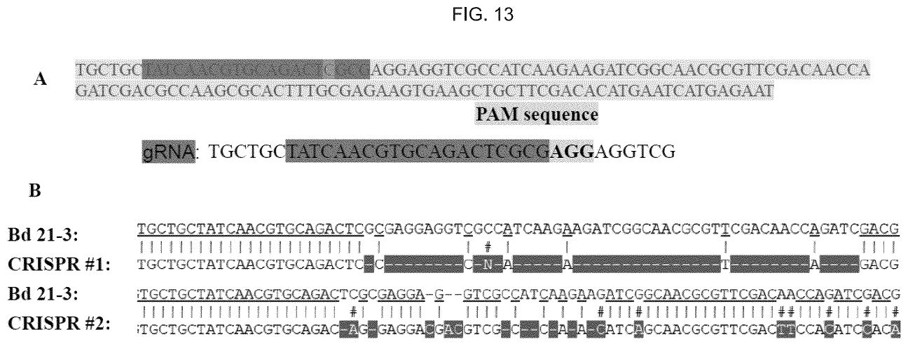

FIG. 13 (or FIG. 7 of Example 2): Design and Sequencing for isolation of chill1 CRISPR plants
(A) Guide RNA design used in generation of CRISPR plants in the Bd 21-3 parent background (highlighted in pink) and followed by the PAM sequence (highlighted in yellow).
(B) Plants generated using this gRNA were sequenced and two of the alleles generated are shown aligned to the parent sequence. Alignment mis-matches between the CRISPR alleles and parent line are highlighted in red showing several small deletions near the PAM sequence.

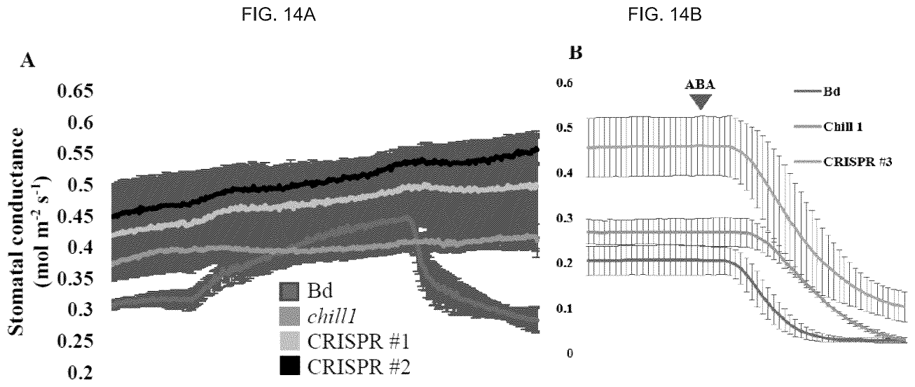

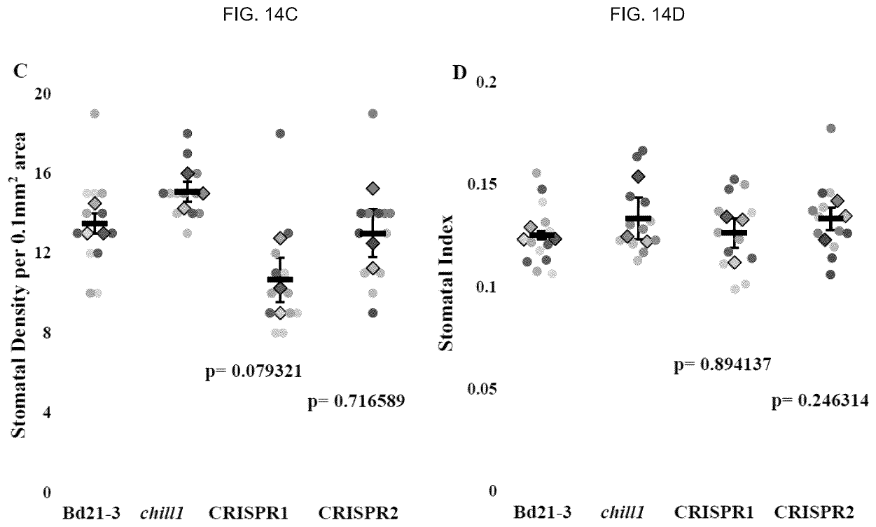

FIG. 14 (or FIG. 8 of Example 2): Bdmpk5 CRISPR Alleles Show Strong CO₂ insensitivity but functional ABA responses.
(A, B) 5 to 6 week old plants were analyzed in time-resolved stomatal conductance analyses.
(A) CRISPR alleles show strongly impaired stomatal responses to CO₂ shifts. Data shown are the average of n=3±SEM experiments using 4 leaves per genotype in each experiment.
(B) CRISPR leaves show functional ABA-induced stomatal closing, despite the large stomatal conductance. Stomatal conductance was recorded for 10 minutes prior to addition of 2 □M ABA. Data shown are the average of n=3±SEM experiments using one leaf per experiment and genotype.
(C,D) Stomatal density (C) and stomatal index (D) were determined using leaf imprints created of the 4^{th} true leaf with 3 replicate plants and genotype. Circles represent counts per each image while diamonds are the average per each plant. Error bars represent SEM while p-values were obtained from two-tailed t-tests comparing mutant to the Bd21-3 parent line (“WT”).

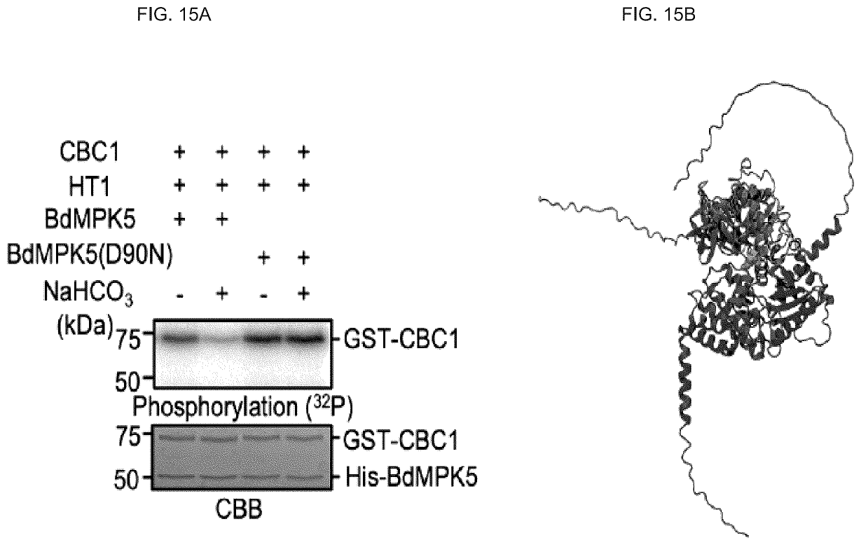

FIG. 15 (or FIG. 9 of Example 2): BdMPK5 functions together with Arabidopsis HT1 and CBC1 in high CO₂/bicarbonate-mediated down-regulation of CBC1 protein kinase phosphorylation and structural prediction of BdMPK5-HT1 complex
(A) In contrast to wildtype BdMPK5 protein, the chill1 BdMPK5 (D90N) variant isoform does not mediate CO₂/HCO3-dependent downregulation of CBC1 kinase phosphorylation.

In vitro phosphorylation assays were performed using GST-CBC1, His-HT1 and His-BdMPK5 (WT or D90N) recombinant proteins with or without 20 mM NaHCO₃. Phosphorylation levels of CBC1 (top) and Coomassie brilliant blue (CBB)-stained gels (bottom) are shown.
(B) AlphaFold2-predicted complex of BdMPK5 with HT1. BdMPK5 is shown in red, while HT1 is shown in blue. The BdMPK5-D90 residue, which is mutant in chill1, is colored green, and is predicted, based on the wildtype BdMPK5-HT1 simulation, to lie at the interface of BdMPK5 and HT1.

FIG. 17A-B illustrate per base sequence quality of read one (FIG. 17A) and read two (FIG. 17B) for the raw sequencing data of the chill1-linked pool consisting of 57 chill1 BC1F2 individuals exhibiting a cooler leaf phenotype. Paired end whole genome sequencing outputs were created in two files for each sample, because all DNA fragments were sequenced in two directions (forward and reverse complement orientation) referred to as read 1 (forward, A) and read 2 (reverse-complement, B). Y-axis represents the phred quality score. Phred quality score is a measure of the quality of base calling generated by automated DNA sequencing. The higher the Phred quality score the more accurate the base calling is. A quality phred score above 28 indicates very high quality (green), between 28 to 20 good quality (orange). A phred quality score below 20 (corresponding to 99% accuracy) is considered to be the cutoff for good sequencing quality (red). The thin dark line (top of A and B) represents the mean quality score at each base position/window. The inner-quartile range for 25th to 75th percentile was so high that range boxes were mostly not visible, with exception of right most read positions. The upper and lower whiskers represent the 10th and 90th percentile scores.

### DISCUSSION

CO₂ regulated stomatal closure can increase water use efficiency (WUE) in grasses by reducing water loss through transpiration (Allen et al., 2011; De Souza et al., 2008; Wang et al., 2015). However, no forward genetic screens of stomatal responses to increased CO₂ in grasses have been reported to date. In the present study, we have developed an unbiased forward genetic screen in the grass B. distachyon to identify mutants showing altered canopy leaf temperature under high and ambient CO₂. Mutant lines were isolated and confirmed throughout three individual stages of infrared screening. Subsequently isolated mutants were screened using real-time stomatal conductance assays. Mutant lines were then taken into the following (M6) generation and investigated in blinded stomatal index and density analyses which showed that the impairments observed were due to stomatal movement responses as opposed to stomatal development. Bulked segregant analyses and resequencing of candidate genes in individual segregating F4 plants and analysis of CRISPR/CAS9-generated bdmpk5 alleles further confirmed mapping of the mutant locus chill1 to the BdMPK5 gene.

Although Arabidopsis thaliana is the most widely used dicot model organism, Brachypodium distachyon is an emerging model organism for monocots due to the increased evolutionary collinearity to important cereal crops such as wheat (Brkljacic et al., 2011, Scholthof et al., 2018, Vogel et al., 2010). Some components of the CO₂ signaling cascade appear to be conserved across monocots and dicots such as bcarbonic anhydrases which have been documented to perform similar functions in stomatal CO₂ responses in A. thaliana, rice, and maize (Chen et al., 2017; Hu et al., 2010; Kolbe et al., 2018). However, stomatal CO₂ sensing mechanisms and the underlying signal transduction pathway remain unknown in grasses. A recent study identified AtMPK4/12 and AtHTI as the two components that together function as primary biosensor for stomatal CO₂ signal transduction controlling stomatal movements in A. thaliana (Takahashi et al., 2022). Our protein sequence analyses show that BdMPK5 is the closest homologue to AtMPK4 and AtMPK12 in A thaliana, further suggesting a level of conservation between CO₂ signaling in both species. This hypothesis is further supporter by findings that BdMPK5 could replace AtMPK4/12 in invitro reconstitution of the “MPK-HT1-CBC1” CO₂ sensing core (FIG. 9A). Interestingly, the chill1 mutant was mapped to a SNP that causes the BdMPK5-D90N variant. Alphafold2 modeling of BdMPk5 wildtype protein with HT1 predicts that this variant lies at the interface of BdMPK5 and HT1 (FIG. 9B), which could explain observed disruption of CO₂ signaling. Considering that Alphafold has sometimes been able to correctly predict the effects of point mutations in some cases (citation: https://www.mdpi.com/1420-3049/27/4/1386 and https://www.biorxiv.org/content/10.1101/2022.10.17.512570v1.abstract and https://www.frontiersin.org/articles/10.3389/fgene.2023.1052383/full #B3 and https://www.biorxiv.org/content/10.1101/2022.04.14.488301v4.abstract) but not in others (citation: https://www.biorxiv.org/content/10.1101/2021.11.03.467194v2.abstract and https://journals.plos.org/plosone/article?id=10.1371/journal.pone.0282689 and https://www.nature.com/articles/s41594-021-00714-2) these results should be treated with caution and will require future structural resolution.

Stomata of grasses are composed of guard cells flanked by lateral subsidiary cells which shrink as turgor increases within guard cells (Franks and Farquhar., 2007; Lawson and Vialet-Chabrand, 2019; McAusland et al., 2016; Nunes et al; 2020; Raschke and Fellows, 1971). This arrangement has been shown to contribute to the increased speed at which grass stomata are able to respond to stimuli thereby improving WUE (Franks and Farquhar., 2007; Lawson and Vialet-Chabrand, 2019; McAusland et al., 2016; Nunes et al; 2020, Raissig et al., 2017). High CO₂ triggers stomatal closure facilitated by ionic efflux out of guard cells leading to a loss of turgor pressure, while subsidiary cells function in direct opposition and increase turgor pressure (Raschke and Fellows; 1971) assisting in the speed and effectivity of stomatal responses (Dubeaux et al., 2021; Farquhar., 2007; Lawson and Vialet-Chabrand, 2019; McAusland et al., 2016; Raschke 1972).

Brachypodium distachyon has a high level of evolutionary similarity to other monocot model species, including maize, wherein the closest homologue, ZmMPK12, has been predicted to be expressed in both guard cells and subsidiary cells, with a much higher expression level within subsidiary cells, based on leaf single cell transcriptomics (Sun et al., 2022). The same study indicates that transcripts of both of the most closely related orthologues of AtHT1 were expressed in maize guard cells but may not be clearly expressed in subsidiary cells (Sun et al., 2022). Due to the increased collinearity between maize and B. distachyon it is possible that the expression patterns may resemble that of maize. Future protein expression studies of BdMPK5 as well as FRET-FLIM analyses in B. distachyon will be needed to determine potential interactions between BdMPK5 and BdHT1. This gives rise to the questions for future studies (1) whether BdHT1 is present only in Brachypodium guard cells or in both guard and subsidiary cell types. (2) Does BdMPK5 contribute to the CO₂ response in both guard cells and subsidiary cells or only in guard cells?

In summary, development of a forward genetics CO₂ response screen in Brachypodium distachyon has led to isolation of stomatal CO₂ response mutants. The chill1 mutant is strongly impaired in stomatal CO₂ responses, but shows robust abscisic acid-induced stomatal closure. Mapping of the chill1 mutant to the BdMPK5 gene, generation and phenotyping of BdMPK5 CRISPR-cas9 alleles and in-vitro reconstitution of the CO₂ sensing core with BdMPK5 suggest that chill1 encodes a component of the CO₂ sensor in grasses. Future mapping of additional mutants and characterization of BdMPK5 in guard cells and subsidiary cells will further dissect stomatal CO₂ sensing and signaling mechanisms in grasses.

### Methods:

### Plant Growth Conditions:

The M5 and subsequent generations of an Ethyl methyl sulfonate (EMS) mutagenized Brachypodium distachyon seed library obtained from the Joint Genome Institute (JGI) were grown alongside the wildtype parent control (Bd21-3). All seeds were cold-treated for a minimum of 5 days at 4° C. prior to sowing. Seeds were sewn in soil containing a mixture of perlite, vermiculite, and Osmocote™ following manufacturer's instructions (3:1:1 soil:perlite:vermiculite). Trays were covered with transparent domes which were removed 7 days following emergence of first true leaves. Plants were grown under 16/8 light/dark conditions under a minimum 200 μE m⁻² s⁻¹ light intensity which has been shown to improve growth conditions. Sequence-indexed sodium azide (NaN) mutagenized lines were grown under the same conditions alongside appropriate controls and the wildtype Bd21-3 parent line.

### Infrared Thermal Imaging Analyses:

4-5 Week old Brachypodium distachyon plants were imaged using an infrared FLIR Thermal Imaging camera T650 sc (FLIR Systems, Inc. Wilsonville, OR 97070 USA). Ambient CO₂ imaging was performed at approximately 450 ppm CO₂ within a plant growth room. Infrared thermal imaging at elevated CO₂ was conducted at a minimum of 900 ppm by placing plants within Percival, E-36HO high CO₂ chambers. Imaging was conducted immediately following removal of plants from high CO₂ chambers before plants were able to equilibrate. Plants were removed from the chamber in small, staggered batches and arranged in groups containing wildtype (Bd 21-3) for comparative analysis. All captured infrared and corresponding parallel bright field images were analyzed individually off-line by at least two laboratory members to reduce bias.

### Stomatal Conductance Analyses:

Healthy 5-6 weeks old plants were selected for gas exchange analyses to measure stomatal conductance (gs) using Licor gas exchange analyzer (LI-6800, LI-COR, Lincoln, NE, USA, or LI-6400XT, LI-COR, Lincoln, NE, USA). Prior to analysis, 4 leaves were bound together using micropore tape (3M), abaxial side downwards (Ceciliato et al., 2019). Leaves were allowed to equilibrate within the analyzer for 1 hour under ambient CO₂ of 400 ppm, 65% humidity, with a light intensity of 250 μmol m⁻² s⁻¹. Data are representative of average data of 3 experiments each using 4 leaves per experiment (minimum total=12 leaves per genotype).

### Microscopy:

Stomatal imaging was performed by creating an imprint of the 4th true leaf from healthy 5-6 week old plants. Leaves were glued onto slides using quick dry super glue to mount the abaxial side to the slide which was then peeled off approximately 45 minutes later once the glue was dry. The resulting imprint was imaged via a compound fluorescence microscope and attached camera. A total of 4 images were taken per leaf at 40× magnification with 3 biological replicates per experiment for a total of 12 images per genotype. Stomatal imaging was done by capturing 2 images above the main vein and 2 below to account for uneven dispersal of stomata. Counting of stomata and pavement cells was done using the “cell-counter” function of IMAGEJ™. Confocal imaging was done on the 4th true leaf after leaves were exposed to a fixing solution of 1:5 35% formaldehyde:ethanol for <2 hours (Sultana et al., 2021). Leaves were then rinsed and stained with 10 mg/mL propidium iodide (PI) for a minimum of 5 minutes before leaves were mounted on slides with water to be imaged using a Leica Confocal Microscope (Raissig 2017). Stomatal aperture was measured manually within FIJI.

Brachypodium distachyon Crossing:

Crossing Brachypodium distachyon plants was performed over a 2-day period in 5-6 week old plants. Individual spikelets containing mature stigma but that had not yet developed mature anthers were used in day 1 and each spikelet was then emasculated. Flowers were taped at the base of the flower with micropore tape (3M) and both anthers were removed using fine pointed forceps without damaging stigma or rupturing anthers. All other flowers were removed from the inflorescence following emasculation. The following day mature anthers were harvested and placed onto a slide inside of a closed plate with a wet paper towel to increase humidity and promote dehiscence. Only anthers that dehisce without manual rupturing are to be used for pollination and were applied to stigma of the emasculated plants from the previous day that did not show any signs of wilting. Plants were then returned to the growth room and kept under well-watered conditions. Seed development should be visible after approximately 4 days.

### Generation of Tillers & Growth Conditions:

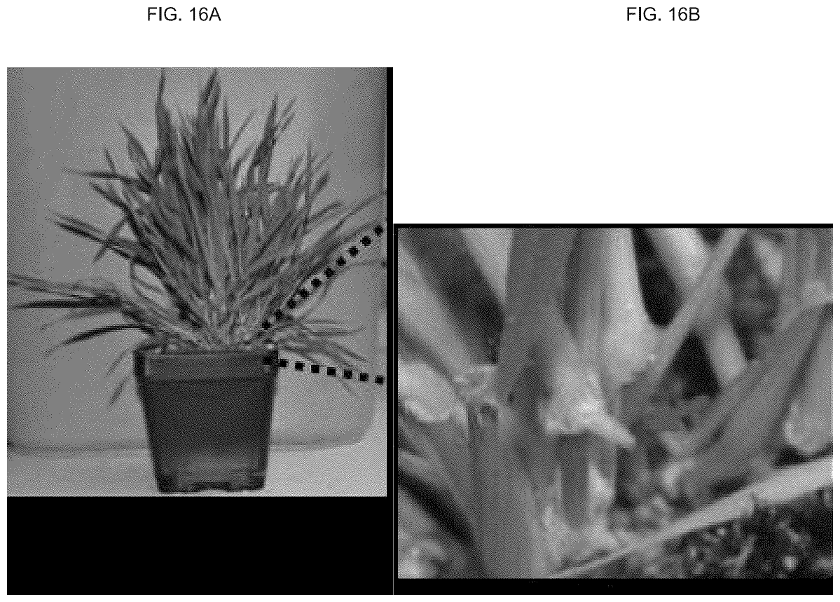

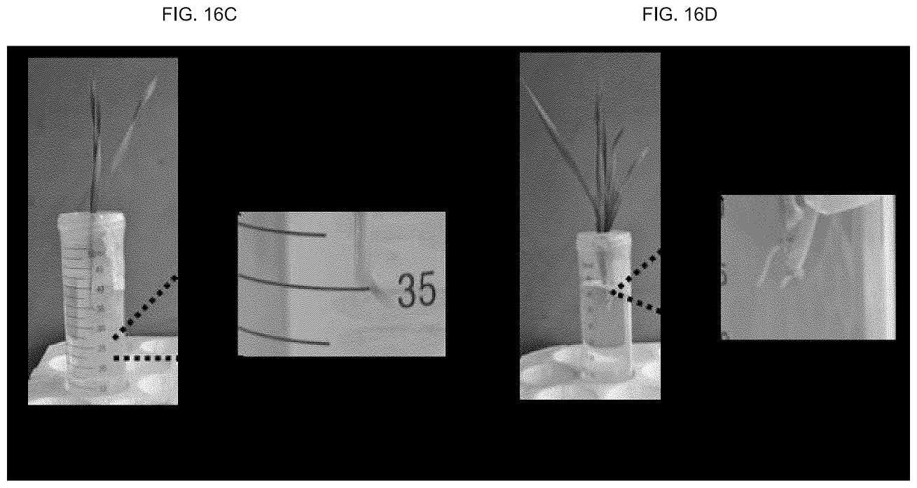

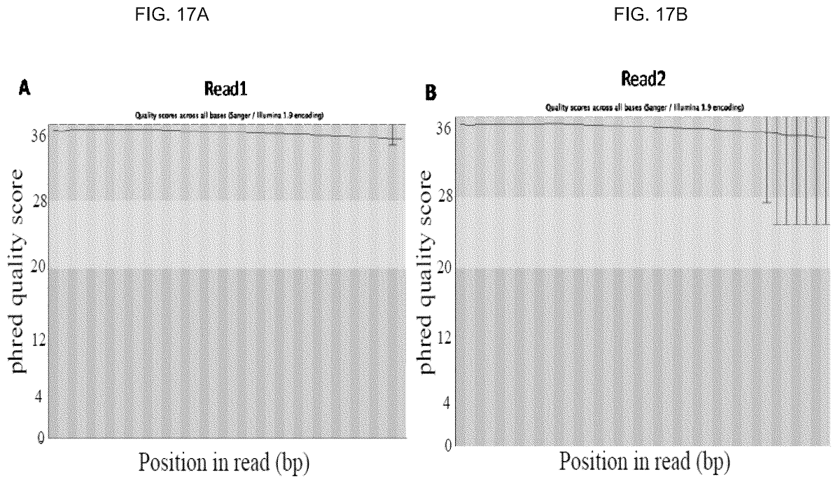

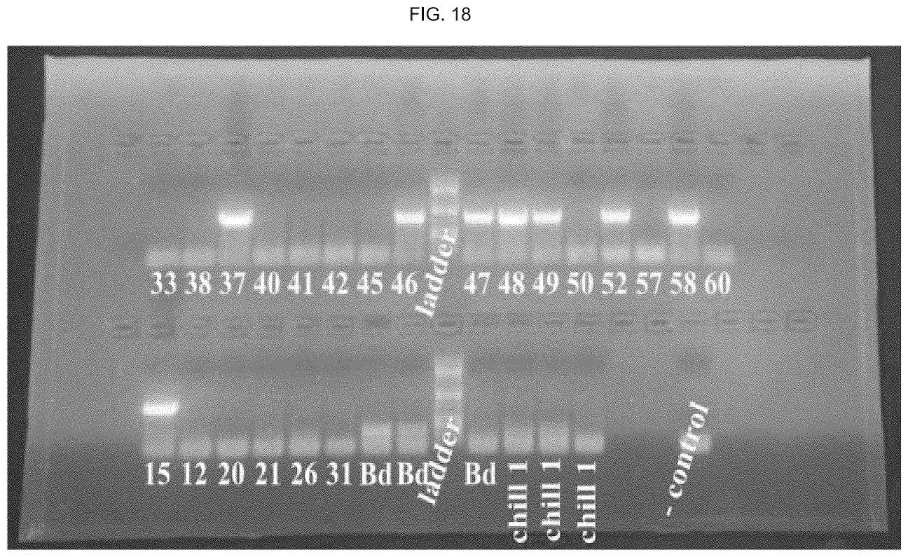

F1 progeny generated by backcrossing to WT (Bd 21-3) were grown under short day (8 hours light/16 hours dark) to promote large growth of plants and aerial root formation. Once aerial root formation could be observed, tillers were created by using a razor blade to make a cut below the node from which the roots grew (FIG. 16). Tillers were immediately placed into Falcon Tubes containing a mix of water and 1× fertilizer solution and grown under short day conditions (8 hours light/16 hours dark). After a minimum of 7 days, cuts were transferred to soil and remained covered with domes to maintain increased humidity for approximately 2 weeks (FIG. 16). Once plants were established in soil, domes were removed and light conditions were switched to long day (22 hours light/2 hours dark) which promotes flowering. F1 tiller populations were allowed to self-cross. F2 seeds were harvested and grown in staggered batches under short day to be used for infrared thermal imaging and subsequent DNA extraction.

### F2 Selection

4-5 week old F2 chill 1 lines backcrossed with the Bd21-3 parent line were analyzed by infrared thermal imaging alongside Bd 21-3 (wildtype) plants following exposure to 1000 ppm CO₂ for 2 hours. Approximately 560 individual plants were screened and images were analyzed independently by 3 people. Plants were sorted based on selections made by laboratory members and were placed into groups to keep only those appearing to have the most robust phenotype. F2 plants selected independently by all 3 persons as showing wildtype-like canopy temperature formed the “wildtype-pool” to be used for comparison to plants with chill1-like phenotypes for Bulked Segregant Analyses (BSA) (Michelmore and Kesseli; 1991). To reduce selection of false positive chill1-like plants all images were screened in a very conservative manner by three independent lab members. Only plants that were selected to be clearly chill1-like by all 3 persons would become the “M” or ‘mutant’ pool. A subset of this pool (SM-pool) selected to be the strongest phenotypically was created by one laboratory member by reanalyzing only those plants which had already been designated as chill1-like by all three persons after individual analyses had been compiled. DNA was extracted from all selected plants using the DNeasy Plant Min Kit (Qiagen, 2016), pooled, and sent out for sequencing via Novogene (Davis, CA). Bulked Segregant Analyses

Sequence data files obtained from Novogene short read whole genome sequencing were first run through quality checks (Ewing and Green, 1998) before proceeding with analysis and all pools were aligned to a Brachypodium distachyon reference sequence obtained via PHYTOZOME™ (https://phytozome-next.jgi.doe.gov/). Sequences were converted from Sequence Alignment Map (SAM) file format to Binary Alignment Map (BAM) before being sorted and indexed using samtools (Li et al, 2009). GATK™ (GENOME ANALYSIS TOOLKIT™) Van der Auwera and O'Connor, 2020; Curr Protoc Bioinformatics, 2013; 43) was then used to mark duplicate sequences, add headers to columns, as well create a BAM index. Before proceeding the reference genome also needed to be prepared before it could be utilized within GATK™ Following this, variants were called using the haplotypecaller function of GATK™ for both M and SM pools and SNPs and indels were separated into 2 separate files for each pool. VARIANTFILTRATION™ was used on all four generated files which were then exported using VARIANTSTOTABLE™ to be used in QTLSEQR™. Variants were annotated using snpEff. The R package QTLSEQR™ was used to import SNP data from GATK™, verify total read depth, check reference allele frequency and the SNP-index as well as filter SNPs. QTLSEQR™ was also used for running the QTLSEQ™ analysis as well as the G′ analysis and generating plots and producing summary tables (Takagi et al, 2013; Magwene et al, 2011).

### Creation of CRISPR Lines

CRISPR lines were designed to induce small deletions in BdMPK5 by generating gRNA with the following oligos: Chill1_CRISPR_Fw (ACTTGTATCAACGTGCAGACTCGCG) (SEQ ID NO:9) and Chill1_CRISPR_Rv (AAACCGCGAGTCTGCACGTTGATAC) (SEQ ID NO:10). Oligos were phosphorylated and utilized in a Golden Gate Reaction to be ligated into CRISPR destination vector JD633 (Addgene). JD633-chill1 plasmid was used to transform E. coli (TOP10) and screened with kanamycin containing media. Destination vector JD633 contains hygromycin resistance as required for transformation by the Boyce Thompson Institute (BTI). Assembled and sequence confirmed plasmids were sent to BTI for transformation in the Bd 21-3 parent background. Lines were screened on plates for hygromycin resistance before shipping. Lines received from BTI were screened again via imaging using the FLIR infrared thermal imaging camera as well as by genotyping to confirm the presence of mutations in the target site. Lines were also genotyped in T2 to confirm the absence of active Cas9 to prevent further sustained off target mutations. All sequencing was done via Sanger Sequencing (Retrogen, San Diego, CA).

### Structural Predictions of BdMPK5 HT1

The BdMPK5-HT1 complex structure was predicted with ALPHAFOLD2, VERSION 2.2.2TM¹ using the multimer functionality2. The source code was downloaded from the ALPHAFOLD2™ Github page (https://github.com/deepmind/alphafold). The sequences of BdMPK5 and HT1 were used for structure prediction, with the D90N mutant made in BdMPK5. We predicted the complex of the long form of HT1 (Uniprot ID: Q2MHE4) with BdMPK5 (Uniprot ID: ???). The maximum template release date we used was from May 14, 2020. We used the full genetic database configuration, and included a final relaxation step on all predicted models. For complex prediction, we created five BdMPK5-HT1 complex models (for example 3 Takahashi et al., 2022), each starting with a random seed, and for each of these models, made five structure predictions. We then ranked all 25 predictions and used the top ranked prediction as our final model. This ranking system uses the predicted template modeling (pTM) score, which may produce a different set of rankings than if one uses the predicted local difference distance test (pLDDT) score to rank structures. However, apart from the manually set maximum template release date, all of these settings are the default ones provided by ALPHAFOLD2™. This workflow and the results are similar to previous predictions of MPK-HT1 complexes3.

BdMPK5 nucleic acid sequences
Bd21-3(WT) BdMPK5 (SEQ ID NO: 11)
(the bolded, underlined, shows the exon
fragment as illustrated in FIG. 11)
(SEQ ID NO: 11)
ATGCGCATGGAAGGCGGCGGCGCCGGGCCGGCGGCAGCAGCAGCAGCAGG
AGGAGCCCATGGCCTCGGCGAGGCGCAGATCAAGGGCACGCTCACCCACG
GCGGCAGGTACGTGCAGTACAACGTCTACGGCAACCTCTTCGAGGTCTCC
GCCAAGTACGTCCCGCCCATCCGTCCCGTCGGCCGCGGCGCCTGCGGCAT
CATCTGTGCTGCTATCAACGTGCAGACTCGCGAGGAGGTCGCCATCAAGA
AGATCGGCAACGCGTTCGACAACCAGATCGACGCCAAGCGCACTTTGCGA
GAAGTGAAGCTGCTTCGACACATGAATCATGAGAATGTGATTTCAATCAA
GGACATCATACGCCCGCCAAGGCGGGAGAATTTTAACGATGTTTACATCG
TTTACGAGCTGATGGACACTGATCTTCACCACCTTCTTAGATCAAACCAG
CCACTCACAGATGATCACTGTCAGTATTTTCTCTACCAAGTGCTCAGAGG
ATTAAAGTATGTGCACTCGGCGAATGTCTTGCACCGAGACCTTAGGCCGA
GCAATCTGTTACTGAATGCCAAATGTGACCTCAAGATTGGGGACTTTGGG
CTGGCGAGGACCACCACTGAGACTGACTTCATGATGGAGTATGTTGTTAC
CCGGTGGTATCGGGCGCCAGAGCTCCTTCTTAACTGCTCGGAGTACACCG
GAGCTATTGATATGTGGTCAGTCGGCTGCATCCTTGGTGAGATTGCAACA
AGAGAGCCTCTGTTTCCTGGAAAAGATTACGTTCATCAGCTGAGGCTAAT
TACCGAGCTGCTAGGCTCACCAGACGACACCAGCCTAGGGTTTCTTCGGA
GCGATAATGCCCGCAGATATGTCAGGTCTCTTCCTCAATACCCCAAGCAA
CAATTTCGTTCACGGTTCCCAAATATGTCCAGTGGCGCCATGGATTTGCT
TGAGAGGATGCTTGTATTTGATCCAAACAAGAGGATTACTGTTGACGAGG
CTCTGTGCCATCCCTACTTGGCATCCCTTCATGAGATAAACGATGAACCT
GTCTGCCCAGCGCCTTTCAGCTTTGATTTCGAGCAGCCATCATTTACCGA
GGAAGATATCAAAGAACTCATTTGGAGGGAATCTGTCAAGTTCAACCCTG
AACCGATTCACTGA
chill1 BdMPK5 (SEQ ID NO: 12)
(the bolded, underlined, shows the exon
fragment as illustrated in FIG. 11)
(SEQ ID NO: 12)
CNNNNNNNGNNGGNGCGCAGCAGCAGCAGGAGGAGCCCATGGCCTCGGCG
AGGCGCAGATCAAGGGCACGCTCACCCACGGCGGCAGGTACGTGCAGTAC
AACGTCTACGGCAACCTCTTCGAGGTCTCCGCCAAGTACGTCCCGCCCAT
CCGTCCCGTCGGCCGCGGCGCCTGCGGCATCATCTGTGCTGCTATCAACG
TGCAGACTCGCGAGGAGGTCGCCATCAAGAAGATCGGCAACGCGTTCAAC
AACCAGATCGACGCCAAGCGCACTTTGCGAGAAGTGAAGCTGCTTCGACA
CATGAATCATGAGAATGTGATTTCAATCAAGGACATCATACGCCCGCCAA
GGCGGGAGAATTTTAACGATGTTTACATCGTTTACGAGCTGATGGACACT
GATCTTCACCACCTTCTTAGATCAAACCAGCCACTCACAGATGATCACTG
TCAGTATTTTCTCTACCAAGTGCTCAGAGGATTAAAGTATGTGCACTCGG
CGAATGTCTTGCACCGAGACCTTAGGCCGAGCAATCTGTTACTGAATGCC
AAATGTGACCTCAAGATTGGGGACTTTGGGCTGGCGAGGACCACCACTGA
GACTGACTTCATGATGGAGTATGTTGTTACCCGGTGGTATCGGGCGCCAG
AGCTCCTTCTTAACTGCTCGGAGTACACCGGAGCTATTGATATGTGGTCA
GTCGGCTGCATCCTTGGTGAGATTGCAACAAGAGAGCCTCTGTTTCCTGG
AAAAGATTACGTTCATCAGCTGANGCTAATTACCGAGCTGCTAGGCTCAC
CAGACGACACCNGCCTAGGGTTTCTTCGGAGCGANNTGCCCGCAGATATN
TCANGTCTCTTCCTCAATACCCCAAGCAACAATTTCGTTCACGGTTCCCA
AATATGTCCNGTGGCGCCCTGGATTTGCTTGAGAGGATGCTTGTATTTGA
TCCAAACAAGAGNATTACTGTTGACGAGGCTCTNTGCCATCCCTACTGGC
ATCCCTTCANGAGATAAACGATNNNNTGTCTGCCCNGCGCCTTTCAGCTT
TGATTTCNAGCAGCCNCNTTTACCGAGNANANNNCNAANNANTCNTTN
BdMPK5 Protein sequences
Bd21-3 (WT) BdMPK5 (SEQ ID NO: 13)
(SEQ ID NO: 13)
MRMEGGGAGPAAAAAAGGAHGLGEAQIKGTLTHGGRYVQYNVYGNLFEVS
AKYVPPIRPVGRGACGIICAAINVQTREEVAIKKIGNAFDNQIDAKRTLR
EVKLLRHMNHENVISIKDIIRPPRRENFNDVYIVYELMDTDLHHLLRSNQ
PLTDDHCQYFLYQVLRGLKYVHSANVLHRDLRPSNLLLNAKCDLKIGDFG
LARTTTETDFMMEYVVTRWYRAPELLLNCSEYTGAIDMWSVGCILGEIAT
REPLFPGKDYVHQLRLITELLGSPDDTSLGFLRSDNARRYVRSLPQYPKQ
QFRSRFPNMSSGAMDLLERMLVFDPNKRITVDEALCHPYLASLHEINDEP
VCPAPFSFDFEQPSFTEEDIKELIWRESVKFNPEPIH*
Chill1 BdMPK5 (SEQ ID NO: 14)
(SEQ ID NO: 14)
MRMEGGGAGPAAAAAAGGAHGLGEAQIKGTLTHGGRYVQYNVYGNLFEVS
AKYVPPIRPVGRGACGIICAAINVQTREEVAIKKIGNAFNNQIDAKRTLR
EVKLLRHMNHENVISIKDIIRPPRRENFNDVYIVYELMDTDLHHLLRSNQ
PLTDDHCQYFLYQVLRGLKYVHSANVLHRDLRPSNLLLNAKCDLKIGDFG
LARTTTETDFMMEYVVTRWYRAPELLLNCSEYTGAIDMWSVGCILGEIAT
REPLFPGKDYVHQLRLITELLGSPDDTSLGFLRSDNARRYVRSLPQYPKQ
QFRSRFPNMSSGAMDLLERMLVFDPNKRITVDEALCHPYLASLHEINDEP
VCPAPFSFDFEQPSFTEEDIKELIWRESVKFNPEPIH*

### REFERENCES EXAMPLE 1

1. E. A. Ainsworth, et al, What have we learned from 15 years of free-air CO2 enrichment (FACE)? A meta-analytic review of the responses of photosynthesis, canopy properties and plant production to rising CO2. New Phytol 165, 351-371 (2005).
2. A. M. Hetherington, et al, The role of stomata in sensing and driving environmental change. Nature 424, 901-908 (2003).
3. J. Negi, M. et al, New approaches to the biology of stomatal guard cells. Plant Cell Physiol 55, 241-250 (2014).
4. Z. Yang et al., Leveraging abscisic acid receptors for efficient water use in Arabidopsis. Proc Natl Acad Sci USA 113, 6791-6796 (2016).
5. J. Zhang et al., Insights into the Molecular Mechanisms of CO2-Mediated Regulation of Stomatal Movements. Curr Biol 28, R1356-R1363 (2018).
6. A. Wu, G. L. Hammer, A. Doherty, S. von Caemmerer, G. D. Farquhar, Quantifying impacts of enhancing photosynthesis on crop yield. Nat Plants 5, 380-388 (2019).
7. H. Hu et al., Carbonic anhydrases are upstream regulators of CO2-controlled stomatal movements in guard cells. Nat Cell Biol 12, 87-93; sup pp 81-18 (2010).
8. A. R. Kolbe, et al, Carbonic Anhydrase Mutants in Zea mays Have Altered Stomatal Responses to Environmental Signals. Plant Physiol 177, 980-989 (2018).
9. S. Xue et al., Central functions of bicarbonate in S-type anion channel activation and OST1 protein kinase in CO2 signal transduction in guard cell. EMBO J 30, 1645-1658 (2011).
10. J. Zhang et al., Identification of SLAC1 anion channel residues required for CO2/bicarbonate sensing and regulation of stomatal movements. Proc Natl Acad Sci USA 115, 11129-11137 (2018).
11. T. Kinoshita, S. Toh, K. U. Torii, Chemical control of stomatal function and development. Curr Opin Plant Biol 60, 102010 (2021).
12. M. Hashimoto et al., Arabidopsis HT1 kinase controls stomatal movements in response to CO₂. Nat Cell Biol 8, 391-397 (2006).
13. A. Hiyama et al., Blue light and CO2 signals converge to regulate light-induced stomatal opening. Nat Commun 8, 1284 (2017).
14. K. Toldsepp et al., Mitogen-activated protein kinases MPK4 and MPK12 are key components mediating CO2-induced stomatal movements. Plant J 96, 1018-1035 (2018).
15. D. Hua et al., A plasma membrane receptor kinase, GHR1, mediates abscisic acid- and hydrogen peroxide-regulated stomatal movement in Arabidopsis. Plant Cell 24, 2546-2561 (2012).
16. M. Sierla et al., The Receptor-like Pseudokinase GHR1 Is Required for Stomatal Closure. Plant Cell 30, 2813-2837 (2018).
17. M. J. Cann, A. Hammer, J. Zhou, T. Kanacher, A defined subset of adenylyl cyclases is regulated by bicarbonate ion. J Biol Chem 278, 35033-35038 (2003).
18. Y. Chen et al., Soluble adenylyl cyclase as an evolutionarily conserved bicarbonate sensor. Science 289, 625-628 (2000).
19. P. D. Townsend et al., Stimulation of mammalian G-protein-responsive adenylyl cyclases by carbon dioxide. J Biol Chem 284, 784-791 (2009).
20. T. Vahisalu et al., SLAC1 is required for plant guard cell S-type anion channel function in stomatal signalling. Nature 452, 487-491 (2008).
21. M. Hashimoto-Sugimoto et al., Dominant and recessive mutations in the Raf-like kinase HT1 gene completely disrupt stomatal responses to CO2 in Arabidopsis. J Exp Bot 67, 3251-3261 (2016).
22. H. Horak et al., A Dominant Mutation in the HT1 Kinase Uncovers Roles of MAP Kinases and GHR1 in CO2-Induced Stomatal Closure. Plant Cell 28, 2493-2509 (2016).
23. L. Jakobson et al., Natural Variation in Arabidopsis Cvi-0 Accession Reveals an Important Role of MPK12 in Guard Cell CO2 Signaling. PLOS Biol 14, e2000322 (2016).
24. G. J. Dow, D. C. Bergmann, J. A. Berry, An integrated model of stomatal development and leaf physiology. New Phytol 201, 1218-1226 (2014).
25. N. M. L. Simon, C. A. Graham, N. E. Comben, A. M. Hetherington, A. N. Dodd, The Circadian Clock Influences the Long-Term Water Use Efficiency of Arabidopsis. Plant Physiol 183, 317-330 (2020).
26. J. Cohen, Statistical Power Analysis. Current Directions in Psychological Science 1, 98-101 (1992).
27. Y. Takahashi et al., MAP3Kinase-dependent SnRK2-kinase activation is required for abscisic acid signal transduction and rapid osmotic stress response. Nat Commun 11, 12 (2020).
28. Y. Yang, et al, Isolation of a strong Arabidopsis guard cell promoter and its potential as a research tool. Plant Methods 4, 6 (2008).
29. Y. Kim, et al, EMS mutagenesis of Arabidopsis. Methods Mol Biol 323, 101-103 (2006).
30. T. Kollist et al., A novel device detects a rapid ozone-induced transient stomatal closure in intact Arabidopsis and its absence in abi2 mutant. Physiologia Plantarum 129, 796-803 (2007).

### REFERENCES—EXAMPLE 2

Allen L H Jr, et al. Elevated CO₂ increases water use efficiency by sustaining photosynthesis of water-limited maize and sorghum. J Plant Physiol. 2011 Nov. 1; 168 (16): 1909-18. doi: 10.1016/j.jplph.2011.05.005. Epub 2011 Jun. 14. PMID: 21676489.
Asseng, S., et al. (2015). Rising temperatures reduce global wheat production. Nature Climate Change, 5 (2), 143-147. https://doi.org/10.1038/nclimate2470
Brkljacic J, et al. Brachypodium as a model for the grasses: today and the future. Plant Physiol. 2011 September; 157 (1): 3-13. doi: 10.1104/pp. 111.179531. Epub 2011 Jul. 19. PMID: 21771916; PMCID: PMC3165879.
Chen, T., Wu, H., Wu, J., Fan, X., Li, X. and Lin, Y. (2017), Absence of OsβCA1 causes a CO2 deficit and affects leaf photosynthesis and the stomatal response to CO2 in rice. Plant J, 90:344-357. https://doi.org/10.1111/tpj.13497
Cingolani P, et al. A program for annotating and predicting the effects of single nucleotide polymorphisms, SnpEff: SNPs in the genome of Drosophila melanogaster strain w1118; iso-2; iso-3. Fly (Austin). 2012 April-June; 6 (2): 80-92. doi: 10.4161/fly.19695. PMID: 22728672; PMCID: PMC3679285.
Dalmais M, et al. A TILLING Platform for Functional Genomics in Brachypodium distachyon. PLOS One. 2013 Jun. 19; 8 (6): e65503. doi: 10.1371/journal.pone.0065503. PMID: 23840336; PMCID: PMC3686759.
De Souza A P, et al. Elevated CO2 increases photosynthesis, biomass and productivity, and modifies gene expression in sugarcane. Plant Cell Environ. 2008 August; 31 (8): 1116-27. doi: 10.1111/j.1365-3040.2008.01822.x. Epub 2008 Apr. 21. PMID: 18433443.
Dubeaux G, et al. Deep dive into CO₂-dependent molecular mechanisms driving stomatal responses in plants. Plant Physiol. 2021 Dec. 4; 187 (4): 2032-2042. doi: 10.1093/plphys/kiab342. PMID: 35142859; PMCID: PMC8644143.
Evans, R; et al, Protein complex prediction with AlphaFold-Multimer. bioRxiv 2022.
Ewing B, Green P. Base-calling of automated sequencer traces using phred. II. Error probabilities. Genome Res. 1998 March; 8 (3): 186-94. PMID: 9521922.
Franks P J, Farquhar G D. The mechanical diversity of stomata and its significance in gas-exchange control. Plant Physiology. 2007; 143:78-87. doi: 10.1104/pp. 106.089367.
Hauser F, et al. A seed resource for screening functionally redundant genes and isolation of new mutants impaired in CO2 and ABA responses. J Exp Bot. 2019 Jan. 7; 70 (2): 641-651. doi: 10.1093/jxb/ery363. PMID: 30346611; PMCID: PMC6322574.
Headquarters, N. (2014). 2014 NASA Strategic Plan. www.nasa.gov
Hu H, et al. Carbonic anhydrases are upstream regulators of CO2-controlled stomatal movements in guard cells. Nat Cell Biol. 2010 January; 12 (1): 87-93; sup pp 1-18. doi: 10.1038/ncb2009. Epub 2009 Dec. 13. Erratum in: Nat Cell Biol. 2011 June; 13 (6): 734. PMID: 20010812; PMCID: PMC2906259.
Hu H, et al. Distinct Cellular Locations of Carbonic Anhydrases Mediate Carbon Dioxide Control of Stomatal Movements. Plant Physiol. 2015 October; 169 (2): 1168-78. doi: 10.1104/pp. 15.00646. Epub 2015 Aug. 4. PMID: 26243620; PMCID: PMC4587455.
Kolbe A R, Brutnell T P, Cousins A B, Studer A J. Carbonic Anhydrase Mutants in Zea mays Have Altered Stomatal Responses to Environmental Signals. Plant Physiol. 2018 July; 177 (3): 980-989. doi: 10.1104/pp. 18.00176. Epub 2018 May 24. PMID: 29794168; PMCID: PMC6053012.
Jakobson L, et al. Natural Variation in Arabidopsis Cvi-0 Accession Reveals an Important Role of MPK12 in Guard Cell CO2 Signaling. PLOS Biol. 2016 Dec. 6; 14 (12): e2000322. doi: 10.1371/journal.pbio.2000322. PMID: 27923039; PMCID: PMC5147794.
Jumper, J; et al, Highly accurate protein structure prediction with AlphaFold. Nature 2021, 596, 583-589. (2021). https://doi.org/10.1038/s41586-021-03819-2
Lawson T, Vialet-Chabrand S. Speedy stomata, photosynthesis and plant water use efficiency. The New Phytologist. 2019; 221:93-98. doi: 10.1111/nph.15330.
Li H, et al; 1000 Genome Project Data Processing Subgroup. The Sequence Alignment/Map format and SAMtools. Bioinformatics. 2009 Aug. 15; 25 (16): 2078-9. doi: 10.1093/bioinformatics/btp352. Epub 2009 Jun. 8. PMID: 19505943; PMCID: PMC2723002.
Magwene P M, Willis J H, Kelly J K (2011) The Statistics of Bulk Segregant Analysis Using Next Generation Sequencing. PLOS Comput Biol 7 (11): e1002255. https://doi.org/10.1371/journal.pcbi.1002255
Mansfeld B N, Grumet R.: An R Package for Bulk Segregant Analysis with Next-Generation Sequencing. Plant Genome. 2018 July; 11 (2). doi: 10.3835/plantgenome2018.01.0006. PMID: 30025013
Marten H, et al. Silencing of NtMPK4 impairs CO-induced stomatal closure, activation of anion channels and cytosolic Casignals in Nicotiana tabacum guard cells. Plant J. 2008 August; 55 (4): 698-708. doi: 10.1111/j.1365-313X.2008.03542.x. Epub 2008 Apr. 30. PMID: 18452588.
Martin, Victoria, et al (2009), Recombinant plant gamma carbonic anhydrase homotrimers bind inorganic carbon, FEBS Letters, 583, doi: 10.1016/j.febslet.2009.09.055
McAusland L, et al. Effects of kinetics of light-induced stomatal responses on photosynthesis and water-use efficiency. The New Phytologist. 2016; 211:1209-1220. doi: 10.1111/nph.14000.
Michelmore R W, et al. Identification of markers linked to disease-resistance genes by bulked segregant analysis: a rapid method to detect markers in specific genomic regions by using segregating populations. Proc Natl Acad Sci USA. 1991 Nov. 1; 88 (21): 9828-32. doi: 10.1073/pnas.88.21.9828. PMID: 1682921; PMCID: PMC52814.
Nunes T D G, Zhang D, Raissig M T. Form, development and function of grass stomata. The Plant Journal. 2020; 101:780-799. doi: 10.1111/tpj.14552.
O'Connor D L, et al. Cross-species functional diversity within the PIN auxin efflux protein family. Elife. 2017 Oct. 24; 6: e31804. doi: 10.7554/eLife.31804. PMID: 29064367; PMCID: PMC5655145.
Peters K, et al. A structural investigation of complex I and I+III2 supercomplex from Zea mays at 11-13 A resolution: assignment of the carbonic anhydrase domain and evidence for structural heterogeneity within complex I. Biochim Biophys Acta. 2008 January; 1777 (1): 84-93. doi: 10.1016/j.bbabio.2007.10.012. Epub 2007 Nov. 4. PMID: 18047828.
Qiao, X., He, Y., Wang, Z. et al. Effect of foliar spray of zinc on chloroplast β-carbonic anhydrase expression and enzyme activity in rice (Oryza sativa L.) leaves. Acta Physiol Plant 36, 263-272 (2014). https://doi.org/10.1007/s11738-013-1407-6
Raissig M T, et al. Mobile MUTE specifies subsidiary cells to build physiologically improved grass stomata. Science. 2017 Mar. 17; 355 (6330): 1215-1218. doi: 10.1126/science.aal3254. Epub 2017 Mar. 16. Erratum in: Science. 2017 Apr. 7; 356 (6333): Gil, MX [corrrected to Anleu Gil, MX]. PMID: 28302860.
Raschke K. Saturation Kinetics of the Velocity of Stomatal Closing in Response to CO(₂). Plant Physiol. 1972 February; 49 (2): 229-34. doi: 10.1104/pp. 49.2.229. PMID: 16657930; PMCID: PMC365934.
Raschke K, Fellows M P. Stomatal movement in Zea mays: Shuttle of potassium and chloride between guard cells and subsidiary cells. Planta. 1971 December; 101 (4): 296-316. doi: 10.1007/BF00398116. PMID: 24488474.
Scholthof K B G, et al. Brachypodium: A Monocot Grass Model Genus for Plant Biology. Plant Cell. 2018 August; 30 (8): 1673-1694. doi: 10.1105/tpc.18.00083. Epub 2018 Jul. 11. PMID: 29997238; PMCID: PMC6139682.
Stocker, T. F., Qin, D., Plattner, G.-K., Tignor, M., Allen, S. K., Boschung, J., Nauels, A., Xia, Y., Bex, V. and Midgley, P. M., Eds. (2013) IPCC, 2013: Climate Change 2013: The Physical Science Basis. Contribution of Working Group I to the Fifth Assessment Report of the Intergovernmental Panel on Climate Change. Cambridge University Press, Cambridge, United Kingdom and New York, NY, USA, 1535 p.
Sultana, S. N.; et al. Optimizing the Experimental Method for Stomata-Profiling Automation of Soybean Leaves Based on Deep Learning. Plants 2021, 10, 2714. https://doi.org/10.3390/plants10122714
Sun G, et al. The maize single-nucleus transcriptome comprehensively describes signaling networks governing movement and development of grass stomata. Plant Cell. 2022 Apr. 26; 34 (5): 1890-1911. doi: 10.1093/plcell/koac047. PMID: 35166333; PMCID: PMC9048877.
Takagi, H., et al. (2013), QTL-seq: rapid mapping of quantitative trait loci in rice by whole genome resequencing of DNA from two bulked populations. Plant J, 74:174-183. doi: 10.1111/tpj.12105
Takahashi Y, et al. Stomatal C O₂/bicarbonate sensor consists of two interacting protein kinases, Raf-like HT1 and non-kinase-activity requiring MPK12/MPK4. Sci Adv. 2022 Dec. 9; 8 (49): eabq6161. doi: 10.1126/sciadv.abq6161. Epub 2022 Dec. 7. PMID: 36475789; PMCID: PMC9728965.
Töldsepp K, et al. Mitogen-activated protein kinases MPK4 and MPK12 are key components mediating CO2-induced stomatal movements. Plant J. 2018 December; 96 (5): 1018-1035. doi: 10.1111/tpj.14087. Epub 2018 Oct. 23. PMID: 30203878; PMCID: PMC6261798.
Van der Auwera G A et al. (2020). Genomics in the Cloud: Using Docker, GATK, and WDL in Terra (1st Edition). O'Reilly Media.
Vogel, J., Bragg, J. (2009). Brachypodium distachyon, a New Model for the Triticeae. In: Muehlbauer, G., Feuillet, C. (eds) Genetics and Genomics of the Triticeae. Plant Genetics and Genomics: Crops and Models, vol 7. Springer, New York, NY. https://doi.org/10.1007/978-0-387-77489-3_16
Vogel J P, et al. 2010. Genome sequencing and analysis of the model grass Brachypodium distachyon. Nature 463, 763-768.
Wahid, A., et al. (2007). Heat tolerance in plants: An overview. In Environmental and Experimental Botany (Vol. 61, Issue 3, pp. 199-223). Elsevier. https://doi.org/10.1016/j.envexpbot.2007.05.011
Wang M, et al. Effects of different elevated CO₂ concentrations on chlorophyll contents, gas exchange, water use efficiency, and PSII activity on C3 and C4 cereal crops in a closed artificial ecosystem. Photosynth Res. 2015 December; 126 (2-3): 351-62. doi: 10.1007/s11120-015-0134-9. Epub 2015 Apr. 14. PMID: 25869633.
Xie, Xiaodong, et al. “The identification of genes involved in the stomatal response to reduced atmospheric relative humidity.” Current Biology 16.9 (2006): 882-887.
Yu S, et al. Expression of a carbonic anhydrase gene is induced by environmental stresses in rice (Oryza sativa L.). Biotechnol Lett. 2007 January; 29 (1): 89-94. doi: 10.1007/s10529-006-9199-z. Epub 2006 Oct. 3. PMID: 17016673.
Zheng, Y., et al. (2019). Elevated CO2 concentration induces photosynthetic down-regulation with changes in leaf structure, non-structural carbohydrates and nitrogen content of soybean. BMC Plant Biology, 19 (1), 255. https://doi.org/10.1186/s12870-019-1788-9
Zhu, Z., et al. Greening of the Earth and its drivers. Nature Clim Change 6, 791-795 (2016). https://doi.org/10.1038/nclimate3004

A number of embodiments of the invention have been described. Nevertheless, it can be understood that various modifications may be made without departing from the spirit and scope of the invention. Accordingly, other embodiments are within the scope of the following claims.

1. A method for:
increasing the water use efficiency of a plant leaf, a plant organ, a plant part or a plant;
increasing the rate of growth or biomass production in a plant leaf, a plant organ, a plant part or a plant;
enhancing the carbon dioxide (CO₂) sensitivity of a guard cell, a plant leaf, a plant organ, plant part or a plant;
down-regulating or decreasing carbon dioxide (CO₂) and/or water exchange in a plant leaf, a plant organ, a plant part or a plant;
decreasing the loss of water, while maintain uptake of carbon dioxide (CO₂) of a plant leaf, a plant organ, plant part or a plant; or
increasing the drought tolerance of a root cell, a plant leaf, a plant organ, a plant part or a plant; or
decreasing the heat resistance or tolerance (optionally decreasing the heat resistance or tolerance under conditions of drought or increased atmospheric carbon dioxide) of a guard cell, a stomatal lineage stage-specific cell, a plant leaf, a plant organ, a plant part or a plant;
comprising:
(a) in the guard cell, stomatal lineage stage-specific cell, plant leaf, plant organ, plant part or plant, increasing the expression and/or protein concentration or protein activity of: a MAP kinase MPK4 and/or a MAP kinase MPK12;
(b) the method of (a), wherein the increasing of expression and/or protein concentration or protein activity of the MAP kinase MPK4 and/or the MAP kinase MPK12, is by:
(1) providing a heterologous a MAP kinase MPK4, a MAP kinase MPK12, or a MAP kinase MPK4-, a MAP kinase MPK12-expressing nucleic acid (optionally a gene, cDNA or message) and expressing the gene, cDNA, message and/or protein in the guard cell, root cell, stomatal lineage stage-specific cell, plant leaf, plant organ, plant part or plant;
(2) increasing of expression and/or protein concentration or protein activity of a homologous a MAP kinase MPK4-, a MAP kinase MPK12-expressing nucleic acid (optionally a gene, cDNA or message); or,
(3) a combination of (1) and (2); or
(c) in a cell of the guard cell, stomatal lineage stage-specific cell, plant leaf, plant organ, plant part or plant, decreasing the expression and/or protein concentration or protein activity of:
(1) a nucleic acid expressing a HT1 kinase; or
(2) a HT1 kinase; or
(d) the method of (a), wherein the decreasing of expression and/or protein concentration or protein activity of the HT1 kinase, is by:
(1) providing a heterologous antisense, iRNA, miRNA or artificial microRNA (miRNA) inhibitory to: the HT1 kinase-encoding nucleic acid (for example, to decrease or abrogate the expression or protein concentration or protein activity of a gene, cDNA or mRNA (message)), or any nucleic acid or compound inhibitory to the expression of the HT1 kinase; and,
expressing the inhibitory nucleic acid or compound, or the heterologous antisense, IRNA, miRNA or artificial microRNA (miRNA), in the guard cell, root cell, stomatal lineage stage-specific cell, plant leaf, plant organ, plant part or plant.

2. A method for:
up-regulating or increasing carbon dioxide (CO₂) and/or water exchange in a guard cell, a root cell, a stomatal lineage stage-specific cell, a plant leaf, a plant organ, a plant part or a plant;
decreasing the water use efficiency of a plant leaf, a plant organ, a plant part or a plant;
decreasing or desensitizing the carbon dioxide (CO₂) sensitivity of a guard cell, a stomatal lineage stage-specific cell, a plant leaf, a plant organ, a plant part or a plant;
upregulating or increasing carbon dioxide (CO₂) and/or water exchange in a plant leaf, a plant organ, a plant part or a plant;
increasing the uptake of CO₂ in a plant leaf, a plant organ, a plant part or a plant;
decreasing drought tolerance in a plant leaf, a plant organ, a plant part or a plant; or
increasing the heat resistance or tolerance (for example, under conditions of drought or increased atmospheric carbon dioxide);
comprising:
(a) in a cell of the guard cell, stomatal lineage stage-specific cell, plant leaf, plant organ, plant part or plant, decreasing the expression and/or protein concentration or protein activity of:
(1) a nucleic acid expressing a MAP kinase MPK4, a MAP kinase MPK12; or
(2) a MAP kinase MPK4, a MAP kinase MPK12;
(b) the method of (a), wherein the decreasing of expression and/or protein concentration or protein activity of a MAP kinase MPK4, a MAP kinase MPK12, is by:
(1) providing a heterologous antisense, IRNA, miRNA or artificial microRNA (miRNA) inhibitory to: the MAP kinase MPK4- and/or a MAP kinase MPK12-encoding nucleic acid (for example, to decrease or abrogate the expression or activity of a gene, cDNA or mRNA (message)), or any nucleic acid or compound inhibitory to the expression of the MAP kinase MPK4 and/or a MAP kinase MPK12; and,
expressing the inhibitory nucleic acid or compound, or the heterologous antisense, iRNA, miRNA or artificial microRNA (miRNA), in the guard cell, root cell, stomatal lineage stage-specific cell, plant leaf, plant organ, plant part or plant;
(2) decreasing of expression and/or activity of a homologous a MAP kinase MPK4-, a MAP kinase MPK12-encoding nucleic acid (optionally a gene, cDNA or mRNA (message)); or,
(3) a combination of (1) and (2); or
(c) expressing or increasing the expression of a HT1-kinase-encoding gene, cDNA or mRNA or transcript, as described in any of the preceding claims, in the plant, guard cell, plant cell, plant leaf, plant organ or plant part.

3. The method of claim 1, wherein the MAP kinase MPK4 polypeptide comprises an amino acid sequence having between about 75% to 100% sequence identity, or 50%, 55%, 60%, 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or complete (100%) sequence identity, with SEQ ID NO:1,
(SEQ ID NO: 1)
MSAESCFGSSGDQSSSKGVATHGGSYVQYNVYGNLFEVSRKYVPPLRPIG
RGAYGIVCAATNSETGEEVAIKKIGNAFDNIIDAKRTLREIKLLKHMDHE
NVIAVKDIIKPPQRENFNDVYIVYELMDTDLHQIIRSNQPLTDDHCRFFL
YQLLRGLKYVHSANVLHRDLKPSNLLLNANCDLKLGDFGLARTKSETDFM
TEYVVTRWYRAPELLLNCSEYTAAIDIWSVGCILGETMTREPLFPGKDYV
HQLRLITELIGSPDDSSLGFLRSDNARRYVRQLPQYPRQNFAARFPNMSA
GAVDLLEKMLVFDPSRRITVDEALCHPYLAPLHDINEEPVCVRPFNFDFE
QPTLTEENIKELIYRETVKFNPQDSV.

4. The method of claim 1, wherein the MAP kinase MPK12 polypeptide comprises an amino acid sequence having between about 75% to 100% sequence identity, or 50%, 55%, 60%, 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or complete (100%) sequence identity, with SEQ ID NO:2,
(SEQ ID NO: 2)
MSGESSSGSTEHCIKVVPTHGGRYVQYNVYGQLFEVSRKYVPPIRPIGRG
ACGIVCAAVNSVTGEKVAIKKIGNAFDNIIDAKRTLREIKLLRHMDHENV
ITIKDIVRPPQRDIFNDVYIVYELMDTDLQRILRSNQTLTSDQCRFLVYQ
LLRGLKYVHSANILHRDLRPSNVLLNSKNELKIGDFGLARTTSDTDFMTE
YVVTRWYRAPELLLNCSEYTAAIDIWSVGCILGEIMTGQPLFPGKDYVHQ
LRLITELVGSPDNSSLGFLRSDNARRYVRQLPRYPKQQFAARFPKMPTTA
IDLLERMLVFDPNRRISVDEALGHAYLSPHHDVAKEPVCSTPFSFDFEHP
SCTEEHIKELIYKESVKFNPDH.

5. The method of claim 1, wherein the HT1 protein kinase polypeptide comprises an amino acid sequence having between about 75% to 100% sequence identity, or 50%, 55%, 60%, 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or complete (100%) sequence identity, with SEQ ID NO:3,
(SEQ ID NO: 3)
MSGLCFNPFRLRWSLRSKLPLEPSLPNLPCNPSSSKTNRYAEAETMEKKR
FDSMESWSMILESENVETWEASKGEREEWTADLSQLFIGNKFASGAHSRI
YRGIYKQRAVAVKMVRIPTHKEETRAKLEQQFKSEVALLSRLFHPNIVQF
IAACKKPPVYCIITEYMSQGNLRMYLNKKEPYSLSIETVLRLALDISRGM
EYLHSQGVIHRDLKSNNLLLNDEMRVKVADFGTSCLETQCREAKGNMGTY
RWMAPEMIKEKPYTRKVDVYSFGIVLWELTTALLPFQGMTPVQAAFAVAE
KNERPPLPASCQPALAHLIKRCWSENPSKRPDFSNIVAVLEKYDECVKEG
LPLTSHASLTKTKKAILDHLKGCVTSISSPFSSSSVPVNA.

6. The method of claim 1, wherein the MAP kinase MPK4-, MAP kinase MPK12-, and/or HT1 protein kinase-expressing nucleic acid (for example, gene, cDNA or mRNA), is operably linked to a plant expressible promoter, an inducible promoter, a constitutive promoter, a root specific promoter, a stomatal lineage stage-specific cell specific promoter, a guard cell specific promoter, a drought-inducible promoter, a stress-inducible promoter or a guard cell active promoter.

7. A method for making a plant with enhanced water use efficiency (WUE), or drought-resistant plant, plant leaf, plant organ or plant part, comprising:
expressing or increasing the expression of a MAP kinase MPK4-, MAP kinase MPK12-encoding gene, cDNA or mRNA or transcript, as described in any of the preceding claims, in the plant, guard cell, plant cell, plant leaf, plant organ or plant part, or
decreasing the expression of an HT1 protein kinase protein-encoding gene or transcript in the plant, guard cell, plant cell, plant leaf, plant organ or plant part, by expressing a nucleic acid inhibitory to the expression of the HT1 protein kinase protein-expressing nucleic acid, gene, cDNA or mRNA or transcript as described in any one of the preceding claims, in the plant, guard cell, plant cell, plant leaf, plant organ, or plant part,
thereby regulating water uptake or water loss and increasing the WUE in the plant, plant cell, plant leaf, plant organ or plant part.

8. A method for making a heat-resistant plant, plant leaf, plant organ, or plant part, comprising:
decreasing the expression of an HT1 protein kinase protein-encoding gene or transcript in the plant, guard cell, plant cell, plant leaf, plant organ or plant part, by expressing a nucleic acid inhibitory to the expression of the HT1 protein kinase protein-expressing nucleic acid, gene, cDNA or mRNA or transcript as described in any one of the preceding claims, in the plant, guard cell, plant cell, plant leaf, plant organ, or plant part, or
expressing or increasing the expression of a MAP kinase MPK4-, MAP kinase MPK12-encoding gene, cDNA or mRNA or transcript, as described in any of the preceding claims, in the plant, guard cell, plant cell, plant leaf, plant organ or plant part,
thereby making a heat-resistant plant, plant leaf, plant organ, or plant part.

9. A method of claim 1, wherein the plant is, or the guard cell, plant cell, plant part or plant organ, is isolated and/or derived from: (i) a dicotyledonous (dicot) or monocotyledonous (monocot) plant; (ii) wheat, oat, rye, barley, rice, sorghum, maize (corn), tobacco, a legume, a lupins, potato, sugar beet, pea, bean, soybean (soy), a cruciferous plant, a cauliflower, rape (or rapa or canola), cane (sugarcane), flax, cotton, palm, sugar beet, peanut, a tree, a poplar, a lupin, a silk cotton tree, desert willow, creosote bush, winterfat, balsa, ramie, kenaf, hemp, roselle, jute, or sisal abaca; or, (c) a species from the genera Anacardium, Arachis, Asparagus, Atropa, Avena, Brassica, Citrus, Citrullus, Capsicum, Carthamus, Cocos, Coffea, Cucumis, Cucurbita, Daucus, Elaeis, Fragaria, Glycine, Gossypium, Helianthus, Heterocallis, Hordeum, Hyoscyamus, Lactuca, Linum, Lolium, Lupinus, Lycopersicon, Malus, Man[iota]hot, Majorana, Medicago, Nicotiana, Olea, Oryza, Panieum, Pannisetum, Persea, Phaseolus, Pistachia, Pisum, Pyrus, Prunus, Raphanus, Ricinus, Secale, Senecio, Sinapis, Solanum, Sorghum, Theobromus, Trigonella, Triticum, Vicia, Vitis, Vigna or Zea.

10. A transgenic guard cell, plant, plant cell, plant tissue, plant seed or fruit, plant part or plant organ, comprising:
a MAP kinase MPK4-, MAP kinase MPK12-, and/or HT1 protein kinase-expressing nucleic acid; or
wherein the nucleic acid, gene or transcript is operably linked to a plant expressible promoter, an inducible promoter, a constitutive promoter, a root specific promoter, a stomatal lineage stage-specific cell specific promoter, a guard cell specific promoter, a drought-inducible promoter, a stress-inducible promoter or a guard cell active promoter;
and the nucleic acid, gene or transcript is stably integrated into the genome of the guard cell, plant, plant cell, plant tissue, plant seed or fruit, plant part or plant organ, or is contained in an episomal vector in the guard cell, plant, plant cell, plant tissue, plant seed or fruit, plant part or plant organ.

11. A transgenic guard cell, plant, plant cell, plant tissue, plant seed or fruit, plant part or plant organ, comprising:
a heterologous nucleic acid that is inhibitory to a MAP kinase MPK4-, MAP kinase MPK12-, and/or HT1 protein kinase-expressing nucleic acid;
wherein the inhibitory nucleic acid is operably linked to a plant expressible promoter, an inducible promoter, a constitutive promoter, a root specific promoter, a stomatal lineage stage-specific cell specific promoter, a guard cell specific promoter, a drought-inducible promoter, a stress-inducible promoter or a guard cell active promoter;
and the inhibitory nucleic acid is stably integrated into the genome of the guard cell, plant, plant cell, plant tissue, plant seed or fruit, plant part or plant organ, or is contained in an episomal vector in the guard cell, plant, plant cell, plant tissue, plant seed or fruit, plant part or plant organ,
and the inhibitory nucleic acid comprises an antisense RNA, siRNA, miRNA or an iRNA or an artificial micro RNA.

12. The method of claim 2, wherein the MAP kinase MPK4 polypeptide comprises an amino acid sequence having between about 75% to 100% sequence identity, or 50%, 55%, 60%, 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or complete (100%) sequence identity, with SEQ ID NO:1,
(SEQ ID NO: 1)
MSAESCFGSSGDQSSSKGVATHGGSYVQYNVYGNLFEVSRKYVPPLRPIG
RGAYGIVCAATNSETGEEVAIKKIGNAFDNIIDAKRTLREIKLLKHMDHE
NVIAVKDIIKPPQRENFNDVYIVYELMDTDLHQIIRSNQPLTDDHCRFFL
YQLLRGLKYVHSANVLHRDLKPSNLLLNANCDLKLGDFGLARTKSETDFM
TEYVVTRWYRAPELLLNCSEYTAAIDIWSVGCILGETMTREPLFPGKDYV
HQLRLITELIGSPDDSSLGFLRSDNARRYVRQLPQYPRQNFAARFPNMSA
GAVDLLEKMLVFDPSRRITVDEALCHPYLAPLHDINEEPVCVRPFNFDFE
QPTLTEENIKELIYRETVKFNPQDSV.

13. The method of claim 2, wherein the MAP kinase MPK12 polypeptide comprises an amino acid sequence having between about 75% to 100% sequence identity, or 50%, 55%, 60%, 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or complete (100%) sequence identity, with SEQ ID NO:2,
(SEQ ID NO: 2)
MSGESSSGSTEHCIKVVPTHGGRYVQYNVYGOLFEVSRKYVPPIRPIGRG
ACGIVCAAVNSVTGEKVAIKKIGNAFDNIIDAKRTLREIKLLRHMDHENV
ITIKDIVRPPQRDIFNDVYIVYELMDTDLQRILRSNQTLTSDQCRFLVYQ
LLRGLKYVHSANILHRDLRPSNVLLNSKNELKIGDFGLARTTSDTDFMTE
YVVTRWYRAPELLLNCSEYTAAIDIWSVGCILGEIMTGQPLFPGKDYVHQ
LRLITELVGSPDNSSLGFLRSDNARRYVRQLPRYPKQQFAARFPKMPTTA
IDLLERMLVFDPNRRISVDEALGHAYLSPHHDVAKEPVCSTPFSFDFEHP
SCTEEHIKELIYKESVKFNPDH.

14. The method of claim 2, wherein the HT1 protein kinase polypeptide comprises an amino acid sequence having between about 75% to 100% sequence identity, or 50%, 55%, 60%, 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or complete (100%) sequence identity, with SEQ ID NO:3,
(SEQ ID NO: 3)
MSGLCFNPFRLRWSLRSKLPLEPSLPNLPCNPSSSKTNRYAEAETMEKKR
FDSMESWSMILESENVETWEASKGEREEWTADLSQLFIGNKFASGAHSRI
YRGIYKQRAVAVKMVRIPTHKEETRAKLEQQFKSEVALLSRLFHPNIVQF
IAACKKPPVYCIITEYMSQGNLRMYLNKKEPYSLSIETVLRLALDISRGM
EYLHSQGVIHRDLKSNNLLLNDEMRVKVADFGTSCLETQCREAKGNMGTY
RWMAPEMIKEKPYTRKVDVYSFGIVLWELTTALLPFQGMTPVQAAFAVAE
KNERPPLPASCQPALAHLIKRCWSENPSKRPDFSNIVAVLEKYDECVKEG
LPLTSHASLTKTKKAILDHLKGCVTSISSPFSSSSVPVNA.

15. The method of claim 2, wherein the MAP kinase MPK4-, MAP kinase MPK12-, and/or HT1 protein kinase-expressing nucleic acid (for example, gene, cDNA or mRNA), is operably linked to a plant expressible promoter, an inducible promoter, a constitutive promoter, a root specific promoter, a stomatal lineage stage-specific cell specific promoter, a guard cell specific promoter, a drought-inducible promoter, a stress-inducible promoter or a guard cell active promoter.

16. The method of claim 7, wherein the increasing of the expression occurs in the plant guard cell or in a precursor cell of the plant guard cell.

17. The method of claim 8, wherein the decreasing of the expression occurs in the plant guard cell or in a precursor of the plant guard cell.

18. The method of claim 2, wherein the plant is, or the guard cell, plant cell, plant part or plant organ, is isolated and/or derived from: (i) a dicotyledonous (dicot) or monocotyledonous (monocot) plant; (ii) wheat, oat, rye, barley, rice, sorghum, maize (corn), tobacco, a legume, a lupins, potato, sugar beet, pea, bean, soybean (soy), a cruciferous plant, a cauliflower, rape (or rapa or canola), cane (sugarcane), flax, cotton, palm, sugar beet, peanut, a tree, a poplar, a lupin, a silk cotton tree, desert willow, creosote bush, winterfat, balsa, ramie, kenaf, hemp, roselle, jute, or sisal abaca; or, (c) a species from the genera Anacardium, Arachis, Asparagus, Atropa, Avena, Brassica, Citrus, Citrullus, Capsicum, Carthamus, Cocos, Coffea, Cucumis, Cucurbita, Daucus, Elaeis, Fragaria, Glycine, Gossypium, Helianthus, Heterocallis, Hordeum, Hyoscyamus, Lactuca, Linum, Lolium, Lupinus, Lycopersicon, Malus, Man[iota]hot, Majorana, Medicago, Nicotiana, Olea, Oryza, Panieum, Pannisetum, Persea, Phaseolus, Pistachia, Pisum, Pyrus, Prunus, Raphanus, Ricinus, Secale, Senecio, Sinapis, Solanum, Sorghum, Theobromus, Trigonella, Triticum, Vicia, Vitis, Vigna or Zea.

19. The method of claim 7, wherein the plant is, or the guard cell, plant cell, plant part or plant organ, is isolated and/or derived from: (i) a dicotyledonous (dicot) or monocotyledonous (monocot) plant; (ii) wheat, oat, rye, barley, rice, sorghum, maize (corn), tobacco, a legume, a lupins, potato, sugar beet, pea, bean, soybean (soy), a cruciferous plant, a cauliflower, rape (or rapa or canola), cane (sugarcane), flax, cotton, palm, sugar beet, peanut, a tree, a poplar, a lupin, a silk cotton tree, desert willow, creosote bush, winterfat, balsa, ramie, kenaf, hemp, roselle, jute, or sisal abaca; or, (c) a species from the genera Anacardium, Arachis, Asparagus, Atropa, Avena, Brassica, Citrus, Citrullus, Capsicum, Carthamus, Cocos, Coffea, Cucumis, Cucurbita, Daucus, Elaeis, Fragaria, Glycine, Gossypium, Helianthus, Heterocallis, Hordeum, Hyoscyamus, Lactuca, Linum, Lolium, Lupinus, Lycopersicon, Malus, Man[iota]hot, Majorana, Medicago, Nicotiana, Olea, Oryza, Panieum, Pannisetum, Persea, Phaseolus, Pistachia, Pisum, Pyrus, Prunus, Raphanus, Ricinus, Secale, Senecio, Sinapis, Solanum, Sorghum, Theobromus, Trigonella, Triticum, Vicia, Vitis, Vigna or Zea.

20. The method of claim 8, wherein the plant is, or the guard cell, plant cell, plant part or plant organ, is isolated and/or derived from: (i) a dicotyledonous (dicot) or monocotyledonous (monocot) plant; (ii) wheat, oat, rye, barley, rice, sorghum, maize (corn), tobacco, a legume, a lupins, potato, sugar beet, pea, bean, soybean (soy), a cruciferous plant, a cauliflower, rape (or rapa or canola), cane (sugarcane), flax, cotton, palm, sugar beet, peanut, a tree, a poplar, a lupin, a silk cotton tree, desert willow, creosote bush, winterfat, balsa, ramie, kenaf, hemp, roselle, jute, or sisal abaca; or, (c) a species from the genera Anacardium, Arachis, Asparagus, Atropa, Avena, Brassica, Citrus, Citrullus, Capsicum, Carthamus, Cocos, Coffea, Cucumis, Cucurbita, Daucus, Elaeis, Fragaria, Glycine, Gossypium, Helianthus, Heterocallis, Hordeum, Hyoscyamus, Lactuca, Linum, Lolium, Lupinus, Lycopersicon, Malus, Man[iota]hot, Majorana, Medicago, Nicotiana, Olea, Oryza, Panieum, Pannisetum, Persea, Phaseolus, Pistachia, Pisum, Pyrus, Prunus, Raphanus, Ricinus, Secale, Senecio, Sinapis, Solanum, Sorghum, Theobromus, Trigonella, Triticum, Vicia, Vitis, Vigna or Zea.
